(19) United States
(12) Patent Application Publication
(10) Pub. No.: US 2022/0081701 A1
(43) Pub. Date: Mar. 17, 2022

(54) DEHYDRATED BIOFILM ASSEMBLIES AND METHODS FOR MANUFACTURING DEHYDRATED BIOFILM ASSEMBLIES

(71) Applicant: STRATIX LABS CORPORATION, ST. PAUL, MN (US)

(72) Inventors: Joshua D. Erickson, BLAINE, MN (US); Mark A. Mulvahill, ST. LOUIS PARK, MN (US)

(73) Assignee: STRATIX LABS CORPORATION, ST. PAUL, MN (US)

(21) Appl. No.: 17/428,788

(22) PCT Filed: Feb. 5, 2020
(86) PCT No.: PCT/US2020/016731

§ 371 Date: Aug. 5, 2021

Related U.S. Application Data
(60) Provisional application No. 62/801,654, filed on Feb. 6, 2019.

(51) Int. Cl. C12Q 1/18; C12N 1/04; C12N 11/02; C12Q 1/04

(52) U.S. Cl. CPC C12Q 1/18; C12N 1/04; C12R 2001/385; C12Q 1/04; C12N 11/02

(57) ABSTRACT

Dehydrated biofilm assemblies and methods for manufacturing dehydrated biofilm assemblies are disclosed. An example dehydrated biofilm assembly may include a dehydrated biofilm assembly for use in antimicrobial testing. The dehydrated biofilm assembly may include a substrate and a dehydrated biofilm secured to the substrate. The dehydrated biofilm may include a viable population of micro-organisms. The dehydrated biofilm may be configured to retain securement to the substrate and exhibit a biofilm phenotype upon hydration.

### CROSS-REFERENCE TO RELATED APPLICATIONS

This application is a National Stage of International Application Serial No. PCT/US2020/016731, filed Feb. 5, 2020, which claims the benefit of priority under 35 U.S.C. § 119 of U.S. Provisional Application Ser. No. 62/801,654, filed Feb. 6, 2019, the entirety of which are incorporated herein by reference.

### TECHNICAL FIELD

The present disclosure pertains to microbiology. More particularly, the present disclosure pertains to biofilms.

### BACKGROUND

Biofilms can have economic, health, and safety impacts.

### BRIEF SUMMARY

This disclosure provides design, material, manufacturing method, and use alternatives for biofilms. A dehydrated biofilm composition is disclosed. The dehydrated biofilm composition comprises: a microbial cell population enmeshed in a self-produced extracellular polymeric matrix and attached to a substrate, wherein the composition retains substrate attachment and exhibits a viable cell count and a biofilm phenotype upon rehydration.

Alternatively or additionally to any of the embodiments above, the composition comprises a viable cell count between about 1×10³ CFU and about 1×10¹² CFU upon rehydration.

Alternatively or additionally to any of the embodiments above, the composition comprises a viable cell count between about 1×10⁵ CFU and about 1×10¹⁰ CFU upon rehydration.

Alternatively or additionally to any of the embodiments above, the composition further comprises a stabilizing agent.

Alternatively or additionally to any of the embodiments above, the composition comprises a stabilizing agent that is selected from the group consisting of: a sugar, a polyol, a polymer, an antioxidant, an amino acid, a surfactant, and a buffer.

Alternatively or additionally to any of the embodiments above, the composition comprises between about 10 mg to about 200 mg of a sugar and between about 5 mg to about 60 mg of an antioxidant.

Alternatively or additionally to any of the embodiments above, the composition further comprises between about 0.1 mg to about 5 mg of a buffer.

Alternatively or additionally to any of the embodiments above, the composition comprises a residual water content of between about 0% to about 15% by weight of the composition.

Alternatively or additionally to any of the embodiments above, the composition comprises a residual water content of between about 0% to about 10% by weight of the composition.

Alternatively or additionally to any of the embodiments above, the composition comprises a residual water content of between about 0% to about 7.5% by weight of the composition.

Alternatively or additionally to any of the embodiments above, the composition exhibits a water activity of less than about 0.9 Aw.

Alternatively or additionally to any of the embodiments above, the composition exhibits a water activity of less than about 0.6 Aw.

Alternatively or additionally to any of the embodiments above, the composition exhibits a water activity of less than about 0.3 Aw.

Alternatively or additionally to any of the embodiments above, the composition has a loss of viability of less than about 3 log CFU over a period of about 30 days at a temperature of about 5° C.

Alternatively or additionally to any of the embodiments above, the composition has a loss of viability of less than about 3 log CFU over a period of about 60 days at a temperature of about 5° C.

Alternatively or additionally to any of the embodiments above, the composition has a loss of viability of less than about 3 log CFU over a period of about 90 days at a temperature of about 5° C.

Alternatively or additionally to any of the embodiments above, the composition comprises an organism selected from the group consisting of: Acinetobacter spp., Aspergillus spp., Bacillus spp., Bordetella spp., Burkholderia spp., Campylobacter spp., Candida spp., Clostridium spp., Corynebacterium spp., Cronobacter spp., Enterobacter spp., Enterococcus spp., Escherichia spp., Haemophilus spp., Klebsiella spp., Legionella spp., Listeria spp., Mycobacterium spp., Peniciullium spp., Proteus spp., Pseudomonas spp., Salmonella spp., Serratia spp., Shigella spp., Staphylococcus spp., Streptococcus spp., Trichophyton spp., Vibrio spp., or Yersinia spp.

Alternatively or additionally to any of the embodiments above, the composition comprises an organism selected from the group consisting of: Acinetobacter baumannii, Aspergillus brasiliensis, Aspergillus niger, Bacillus cereus, Bacillus subtilis, Bordetella pertussis, Burkholderia cepacia, Campylobacter jejuni, Candida albicans, Candida auris, Clostridium difficile, Clostridium sporogenes, Clostridium botulinum, Corynebacterium ammoniagenes, Cronobacter sakazakii, Enterobacter aerogenes, Enterobacter cloacae, Enterococcus faecalis, Escherichia coli, Haemophilus influenzae, Klebsiella oxytoca, Klebsiella pneumoniae, Legionella pneumophila, Listeria monocytogenes, Mycobacterium bovis, Mycobacterium terrae, Peniciullium chrysogenum, Proteus mirabilis, Pseudomonas aeruginosa, Salmonella bongori, Salmonella enterica, Serratia marcescens, Shigella dysenteriae, Shigella flexneri, Shigella sonnei, Staphylococcus aureus, Staphylococcus epidermidis, Streptococcus pneumoniae, Streptococcus pyogenes, Trichophyton mentagrophytes, Vibrio cholera, Yersinia enterocolitica, or a strain thereof.

Alternatively or additionally to any of the embodiments above, the composition comprises an organism selected from the group consisting of: Acinetobacter baumannii (ATCC 19606), Aspergillus brasiliensis (ATCC 16404), Aspergillus niger (ATCC 6275), Aspergillus niger (ATCC 16404), Bordetella pertussis (ATCC 12743), Campylobacter jejuni (ATCC 33291), Campylobacter jejuni (ATCC 29428), Candida albicans (ATCC 10231), Clostridium difficile (ATCC 43598), Cronobacter sakazakii (ATCC 12868), Enterobacter aerogenes (ATCC 13048), Enterococcus faecalis (ATCC 29212), Escherichia coli (ATCC 10536), Escherichia coli (ATCC 11229), Escherichia coli O157:H7 (ATCC 35150), Haemophilus influenzae (ATCC 10211), Klebsiella oxytoca (ATCC 13182), Klebsiella pneumoniae (ATCC 4352), Legionella pneumophila (ATCC 33153), Listeria monocytogenes (ATCC 19117), Listeria monocytogenes (ATCC 19111), Listeria monocytogenes (ATCC 7644), Methicillin Resistant Staphylococcus aureus (ATCC BAA-1683), Methicillin Resistant Staphylococcus aureus (ATCC 33592), Multi-drug Resistant Enterococcus faecium (ATCC 51559), Multi-drug resistant Klebsiella pneumoniae (ATCC 51503), Mycobacterium bovis (ATCC 35743), Mycobacterium terrae (ATCC 15755), Proteus mirabilis (ATCC 9240), Pseudomonas aeruginosa (ATCC 15442), Salmonella enterica (ATCC 10708), Salmonella enterica (ATCC 6539), Serratia marcescens (ATCC 14756), Shigella dysenteriae (ATCC 11835), Shigella flexneri (ATCC 29508), Shigella sonnei (ATCC 11060), Staphylococcus aureus (ATCC 6538), Staphylococcus epidermidis (ATCC 12228), Streptococcus pneumoniae (ATCC 6305), Streptococcus pyogenes (ATCC 12384), Streptococcus pyogenes (ATCC 19615), Trichophyton interdigitale (ATCC 9533), Trichophyton mentagrophytes (ATCC 9533), Vancomycin Resistant Enterococcus faecalis (ATCC 51575), Vibrio cholera (ATCC 11623), or Yersinia enterocolitica (ATCC 23715).

Alternatively or additionally to any of the embodiments above, the composition is enclosed within a primary container.

Alternatively or additionally to any of the embodiments above, the primary container is sealed within a secondary container that has low permeability to moisture and oxygen.

Alternatively or additionally to any of the embodiments above, the primary container or secondary container is impermeable to moisture and oxygen.

Alternatively or additionally to any of the embodiments above, a desiccant is included within the secondary container.

Alternatively or additionally to any of the embodiments above, an oxygen scavenger is included within the secondary container.

Alternatively or additionally to any of the embodiments above, the composition is dehydrated by lyophilization.

Alternatively or additionally to any of the embodiments above, the composition is prepared by a process that includes: (1) growing a biofilm composition on a substrate, (2) adding a stabilizing agent to the biofilm composition, (3) freezing the biofilm composition, (4) dehydrating the biofilm composition by sublimation, (5) dehydrating the biofilm composition by desorption, and (6) enclosing the dehydrated biofilm composition in a primary container.

Alternatively or additionally to any of the embodiments above, the composition is used to evaluate the anti-biofilm efficacy of a test substance.

Alternatively or additionally to any of the embodiments above, the anti-biofilm efficacy of the test substance is evaluated by a process that includes: (1) submerging a dehydrated biofilm composition in an aqueous solution, (2) maintaining the composition in the aqueous solution for an amount of time sufficient for rehydration, (3) contacting the rehydrated biofilm composition with a test substance for a contact time, (4) performing an assay to determine the anti-biofilm efficacy of the test substance.

Alternatively or additionally to any of the embodiments above, evaluating the anti-biofilm efficacy of the test substance includes evaluating the ability of a test substance to remove a biofilm from a substrate or the ability to kill microbes in a biofilm state.

A kit for evaluating the anti-biofilm efficacy of a test substance is disclosed. The kit comprises: a dehydrated biofilm composition comprising a microbial cell population enmeshed in a self-produced extracellular polymeric matrix and attached to a substrate, wherein the composition retains substrate attachment and exhibits a viable cell count and a biofilm phenotype upon rehydration, wherein the dehydrated biofilm composition comprises a viable cell count between about 1×10³ CFU and about 1×10¹² CFU upon rehydration, wherein the dehydrated biofilm composition comprises a stabilizing agent, wherein the dehydrated biofilm composition comprises a residual water content of between about 0% to about 10% by weight of the composition, wherein the dehydrated biofilm composition has a loss of viability of less than 3 log CFU over a period of about 90 days at a temperature of about 5° C., wherein the dehydrated biofilm composition is enclosed within a primary container, and wherein the primary container is sealed within a secondary container that has low permeability to moisture and oxygen.

A dehydrated biofilm assembly for use in antimicrobial testing is disclosed. The dehydrated biofilm assembly comprises: a substrate; a dehydrated biofilm secured to the substrate; wherein the dehydrated biofilm includes a viable population of micro-organisms; and wherein the dehydrated biofilm is configured to retain securement to the substrate and exhibit a biofilm phenotype upon rehydration.

Alternatively or additionally to any of the embodiments above, the viable population of micro-organisms is disposed in a matrix.

Alternatively or additionally to any of the embodiments above, the viable population of micro-organisms comprises a viable cell count in the range of about 1×10⁶ CFU and about 1×10¹⁰ CFU of micro-organisms.

Alternatively or additionally to any of the embodiments above, the viable population of micro-organisms comprises Pseudomonas aeruginosa.

Alternatively or additionally to any of the embodiments above, the viable population of micro-organisms comprises a viable cell count in the range of about 1×10⁸ CFU and about 1×10^{9.5} CFU Pseudomonas aeruginosa.

Alternatively or additionally to any of the embodiments above, the viable population of micro-organisms comprises Staphylococcus aureus.

Alternatively or additionally to any of the embodiments above, the viable population of micro-organisms comprises a viable cell count in the range of about 1×10^{7.5} CFU and about 1×10⁹ CFU Staphylococcus aureus.

Alternatively or additionally to any of the embodiments above, the dehydrated biofilm includes a stabilizing agent.

Alternatively or additionally to any of the embodiments above, the stabilizing agent includes a sugar.

Alternatively or additionally to any of the embodiments above, the stabilizing agent includes a non-reducing sugar.

Alternatively or additionally to any of the embodiments above, the stabilizing agent includes sucrose.

Alternatively or additionally to any of the embodiments above, the stabilizing agent includes an antioxidant.

Alternatively or additionally to any of the embodiments above, the stabilizing agent includes ascorbic acid.

Alternatively or additionally to any of the embodiments above, the stabilizing agent includes a buffer.

Alternatively or additionally to any of the embodiments above, the stabilizing agent includes Tris(hydroxymethyl)aminomethane.

Alternatively or additionally to any of the embodiments above, the dehydrated biofilm has a residual water content in the range of about 0-10% by weight of the dehydrated biofilm.

Alternatively or additionally to any of the embodiments above, the dehydrated biofilm has a residual water content in the range of about 0-7.5% by weight of the dehydrated biofilm.

Alternatively or additionally to any of the embodiments above, the dehydrated biofilm has a loss of viability of less than about 3 log CFU over a period of 30 days at a temperature of 5° C.

Alternatively or additionally to any of the embodiments above, the dehydrated biofilm has a loss of viability of less than about 3 log CFU over a period of 60 days at a temperature of 5° C.

Alternatively or additionally to any of the embodiments above, the dehydrated biofilm has a loss of viability of less than about 3 log CFU over a period of 90 days at a temperature of 5° C.

Alternatively or additionally to any of the embodiments above, the dehydrated biofilm has a loss of viability of less than about 0.5 log CFU over a period of 30 days at a temperature of 5° C.

Alternatively or additionally to any of the embodiments above, the dehydrated biofilm has a loss of viability of less than about 0.5 log CFU over a period of 60 days at a temperature of 5° C.

Alternatively or additionally to any of the embodiments above, the dehydrated biofilm has a loss of viability of less than about 0.5 log CFU over a period of 90 days at a temperature of 5° C.

Alternatively or additionally to any of the embodiments above, the dehydrated biofilm and the substrate are disposed within a primary container.

Alternatively or additionally to any of the embodiments above, the primary container is sealed within a secondary container that has low permeability to moisture and oxygen.

Alternatively or additionally to any of the embodiments above, the primary container, the secondary container, or both are impermeable to at least one of moisture and oxygen.

Alternatively or additionally to any of the embodiments above, further comprising a desiccant disposed within the secondary container.

Alternatively or additionally to any of the embodiments above, further comprising an oxygen scavenger disposed within the secondary container.

A method for testing the anti-biofilm efficacy of a test substance is disclosed. The method comprises: submerging a dehydrated biofilm assembly in an aqueous solution, the dehydrated biofilm assembly including a dehydrated biofilm secured to a substrate; disposing the dehydrated biofilm assembly in the aqueous solution for a rehydration time to form a rehydrated biofilm; contacting the rehydrated biofilm with a test substance for a contact time; contacting the rehydrated biofilm and the test substance with a neutralizer; agitating the rehydrated biofilm; and observing an indication of anti-biofilm efficacy.

Alternatively or additionally to any of the embodiments above, observing an indication of anti-biofilm efficacy includes quantitative assessment.

Alternatively or additionally to any of the embodiments above, quantitative assessment includes determining a loss of viable cells in the rehydrated biofilm.

Alternatively or additionally to any of the embodiments above, quantitative assessment includes utilizing a stain or a dye.

Alternatively or additionally to any of the embodiments above, observing an indication of anti-biofilm efficacy includes qualitative assessment.

Alternatively or additionally to any of the embodiments above, the rehydration time is about 1-40 minutes.

Alternatively or additionally to any of the embodiments above, the rehydration time is about 1-25 minutes.

Alternatively or additionally to any of the embodiments above, the test substance includes a disinfectant.

Alternatively or additionally to any of the embodiments above, the contact time is about 1-10 minutes.

Alternatively or additionally to any of the embodiments above, the dehydrated biofilm assembly is disposed in a container and wherein disposing the dehydrated biofilm assembly in the aqueous solution for a rehydration time to form a rehydrated biofilm further includes removing the rehydrated biofilm from the container.

A method for manufacturing a dehydrated biofilm assembly for use in antimicrobial testing is disclosed. The method comprises: culturing a biofilm on a substrate; submerging the biofilm in a solution that includes a stabilizing agent; freezing the biofilm in the solution; lyophilizing the biofilm to dehydrate the biofilm; disposing the dehydrated biofilm and the substrate in a primary container; and sealing the primary container within a secondary container that has low permeability to moisture and oxygen.

Alternatively or additionally to any of the embodiments above, the primary container, the secondary container, or both are impermeable to at least one of moisture and oxygen.

A kit for evaluating the anti-biofilm efficacy of a test substance is disclosed. The kit comprises: a dehydrated biofilm composition comprising a microbial cell population enmeshed in a self-produced extracellular polymeric matrix and attached to a substrate, wherein the composition retains substrate attachment and exhibits a viable cell count and a biofilm phenotype upon rehydration, wherein the viable cell count is about 1×10⁶ CFU and about 1×10¹⁰ CFU upon rehydration of the dehydrated biofilm composition, wherein the dehydrated biofilm composition comprises a stabilizing agent, wherein the dehydrated biofilm composition comprises a residual water content of between about 0% to about 10% by weight of the dehydrated biofilm composition, wherein the dehydrated biofilm composition has a loss of viability of less than 3 log CFU over a period of about 90 days at a temperature of about 5° C., wherein the dehydrated biofilm composition is disposed within a primary container, and wherein the primary container is sealed within a secondary container that has low permeability to moisture and oxygen.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example substrate.

FIG. 2 is a top view of an example substrate.

FIG. 3 illustrates an example substrate.

FIG. 4 illustrates an example substrate.

FIG. 5 is a top view of an example substrate.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

As used herein, the term “or” is inclusive and means any one element of a particular list, and includes any combination of elements of that list. For example, the phrase “A or B” means “A, B, or A and B”. Thus “or” should be interpreted as “and/or” even if “and/or” is not explicitly used.

The terms “microbe” or “microorganism” may be understood to include prokaryotic and eukaryotic microbial species from the Domains Archaea, Bacteria and Eucarya, the latter including yeast and filamentous fungi, protozoa, algae, or higher Protista, as well as viruses. The terms “microbial cells” and “microbes” are used interchangeably with the term “microorganism”.

The term “microbial cell population” may be understood to refer to an aggregate, group, or cluster of more than one microbial cell.

The term “enmeshed” in the context of microorganisms in a biofilm state may be understood to mean embedded in, disposed in, surrounded by, or grouped with. For example, the phrase “a microbial cell population enmeshed in an extracellular polymeric matrix” may be understood to mean “a microbial cell population disposed in an extracellular polymeric matrix”.

The term “extracellular polymeric matrix” in the context of a biofilm may be understood to mean a structure of polymer substances secreted by microorganisms. In some examples, an extracellular polymeric matrix can further include proteins, extracellular DNA, or other compounds.

The term “attached” or “attachment” may be understood to mean being adhered or connected. For example, the phrase “a biofilm attached to a substrate” may be understood to mean “a biofilm adhered to a substrate”.

The term “retains” may be understood to mean to continue to have something. For example, the phrase “a dehydrated biofilm composition retains substrate attachment upon rehydration” may be understood to mean “a dehydrated biofilm composition continues to have substrate attachment upon rehydration”.

The term “exhibits” may be understood to mean to have a quality, feature, or characteristic. For example, the phrase “exhibits a viable cell count and a biofilm phenotype” may be understood to mean “has a viable cell count and has a biofilm phenotype”.

The term “viable cell count” may be understood to mean the number of cells that are alive and capable of regeneration and/or propagation.

The term “loss of viability” in the context of a dehydrated biofilm composition may be understood to refer to a reduction in the viable cell count of a composition over a given period of time (e.g., when stored at a certain temperature). It may be understood that this is relative to the viable cell count of a dehydrated biofilm composition prepared using the same methodology (e.g., the same organism strain grown using the same method and preserved using the same composition of stabilizing agents and dehydration process) as calculated immediately following dehydration.

The term “biofilm” may be understood to mean a microbial cell population embedded in a matrix of self-produced extracellular polymeric substances. A biofilm is a self-organized community of microbes that behaves differently than free floating planktonic microbes. A biofilm can be attached to a substrate or to itself. A biofilm may form at a non-solid interface (e.g., an air-water interface).

The term “biofilm phenotype” may be understood to mean a microbial cell population enmeshed in a self-produced extracellular polymeric matrix that exhibit observable characteristics distinct from free floating planktonic microbes of the same species or strain, e.g., differences in gene expression, metabolism, growth rates, antimicrobial tolerance, or other behaviors.

The term “substrate” in the context of a biofilm composition may be understood to mean the underlying surface or material which a biofilm is adhered to. The terms “coupon” and “carrier” can be used interchangeably with the term “substrate”.

The terms “dehydrated,” “dehydration,” or “dehydrating” may be understood to refer to the removal of water, for example, the removal of 5-15%, 15-35%, 35-50%, 50-75%, 75-90%, or 90-100% of water.

The term “dehydrated biofilm composition” may be understood to refer to a biofilm composition where an amount of water has been removed. The term “dehydrated biofilm assembly” can be used interchangeably with the term “dehydrated biofilm composition”

The terms “rehydrated,” “rehydration,” or “rehydrating” may be understood to refer to adding water to a dehydrated composition. The terms “reconstituted,” “reconstitution,” and “reconstituting” can be used interchangeably with the terms “rehydrated,” “rehydration,” and “rehydrating,” respectively.

The term “rehydrated biofilm composition” may be understood to mean a biofilm composition that was previously dehydrated and was subsequently rehydrated. The term “reconstituted biofilm composition” is used interchangeably with the term “rehydrated biofilm composition”.

The term “lyophilization” may be understood to refer to the process of dehydrating a material or a composition using a series of steps that include freezing the composition, lowering pressure, and removing ice by sublimation. Lyophilization can further include one or more steps involving dehydration by desorption.

The term “lyophilizer” or “freeze dryer” may be understood to refer to equipment used to dehydrate a material or a composition by lyophilization.

The term “lyophilized” may be understood to refer to any solid material or composition obtained by the process of lyophilization. The terms “freeze dried” and “freeze-dried” can be used interchangeably with the term “lyophilized”.

The term “residual water content” may be understood to refer to the amount of water remaining in a dehydrated composition. It can be referred to as a percent of the weight of the composition or as a quantified amount of mass. The term “water content” can be used interchangeably with the term “residual water content”.

The term “moisture” may be understood to refer to unbound water and any other volatile substances that can be evaporated from a composition.

The term “residual moisture content” may be understood to refer to the amount of moisture remaining in a dehydrated composition. It can be referred to as a percent of the weight of the composition or as a quantified amount of mass. Residual moisture content is closely related to residual water content and in some compositions may be the same value as the residual water content. The term “moisture content” can be used interchangeably with the term “residual moisture content”.

The term “water activity” (Aw) may be understood to refer to the availability of water and represents the energy status of the water in a system. It is defined as the vapor pressure of water above a sample divided by that of pure water at the same temperature. Pure distilled water has a water activity of exactly one (e.g., Aw=1.0).

The term “disc” may be understood to refer to a flat, thin, and round object. The term “disk” can be used interchangeably with the term “disc”.

The term “microcarrier” may be understood to refer to a substrate suitable the cultivation and adherence of biofilm that is typically spherical in shape and has a diameter between about 10 microns to about 5 mm.

The term “penicylinder” may be understood to refer to a small, hollow cylinder used in scientific experiments evaluating antimicrobial activity. The term “cylinder” can be used interchangeably with the term “penicylinder” in the context of scientific experiments evaluating antimicrobial activity.

The term “reservoir” or “reservoir compartment” may be understood to refer to a receptacle capable of holding fluid.

The term “test tube” may be understood to refer to a tube closed at one end that is capable of holding fluid.

The term “vial” may be understood to refer to a small container, typically cylindrical in shape that is capable of holding fluid.

The term “sufficient” in the context of a quantity may be understood to refer to an adequate amount to achieve the desired effect. For example, the phrase “maintaining the composition in an aqueous solution for an amount of time sufficient for rehydration” may be understood to mean “maintaining the composition in an aqueous solution for a duration of time that is long enough to rehydrate the composition”.

The term “effective amount” may be understood to refer to an adequate quantity to achieve the desired effect. For example, the phrase “an effective amount of a stabilizing agent” may be understood to mean “a quantity of a stabilizing agent that improves the stability of the composition”.

The term “stabilizing agent” may be understood to refer to a chemical or compound that is included in a composition in order to improve the retention of a desired characteristic (e.g., cell viability) during and after the dehydration process. It is understood that this is relative to the characteristics of a similarly prepared dehydrated composition that lacks the stabilizing agent(s). A stabilizing agent may improve the retention of a desired characteristic during any stage of the dehydration process (e.g., freezing, sublimation, or desorption) or after dehydration (e.g., during storage over a period of time). A stabilizing agent may act as one or more of the following: a lyoprotectant, a cryoprotectant, a buffering agent, an antioxidant, or a bulking agent.

The terms “antioxidant” or “anti-oxidizing agent” may be understood to refer to a chemical or compound that acts to slow down, reduce, inhibit or prevent oxidation, e.g., in a dehydrated biofilm composition.

The terms “buffer,” “buffering agent,” or “buffering solution” may be understood to refer to a weak acid or base that is used to maintain the pH of a solution or composition near a desired pH value. For example, a buffering agent is able to resist or diminish changes in pH when an acid or base is added to the solution or composition.

The terms “lyoprotectant” or “lyoprotecting agent” may be understood to refer to a chemical or compound that is included in a composition in order to improve the retention of a desired characteristic (e.g., cell viability) during lyophilization.

The terms “cryoprotectant” or “cryoprotecting agent” may be understood to refer to a chemical or compound that is included in a composition in order to improve the retention of a desired characteristic (e.g., cell viability) during freezing.

The term “bulking agent” may be understood to refer to a chemical or compound that is included in a composition in order to add bulk to the dehydrated composition and/or assist in the control of the properties of the formulation during dehydration.

The term “container” may be understood to refer to an object that can be used to hold or transport a composition (e.g., a dehydrated biofilm composition).

The term “primary container” may be understood to refer to a container that is in direct contact with a composition (e.g., a dehydrated biofilm composition).

The term “enclosed” may be understood to mean surrounded or closed off on all sides. For example, the phrase “a dehydrated biofilm composition enclosed in a primary container” represents “a dehydrated biofilm composition in direct contact with a container that is closed off on all sides”.

The term “secondary container” may be understood to refer to a container that is in direct contact with a primary container and is not in direct contact with a composition (e.g., a dehydrated biofilm composition).

The term “permeability” in the context of a container may be understood to refer to the quality or state of the container material to allow a liquid or gas (e.g., moisture vapor or oxygen) to pass through it.

The term “low permeability” in the context of a container may be understood to refer to the quality or state of the container material to act as a barrier and to lessen the quantity of a liquid or gas (e.g., moisture vapor or oxygen) that passes through it, e.g., over a period of time. As used herein, a container with “low permeability to moisture” is defined as having a moisture vapor transmission rate (MVTR) of <1.0 g/100 in²/24 hours at 38° C. and 90% relative humidity, as measured by an applicable standardized test method (e.g., ASTM F1249 for plastic films). As used herein, a container with “low permeability to oxygen” is defined as having an oxygen transmission rate (OTR) of <1.0 cc/100 in²/24 hours at 23° C. and 90% relative humidity, as measured by an applicable standardized test method (e.g., ASTM D3985 for plastic films). Other methods for measuring MVTR and OTR may be utilized.

The term “impermeable” in the context of a container may be understood to refer to the quality of state of the container material to block substantially all liquid and gas from passing through it. As used herein, a container that is “impermeable to moisture” is defined as having a moisture vapor transmission rate (MVTR) of <0.1 g/100 in²/24 hours at 38° C. and 90% relative humidity, as measured by an applicable standardized test method (e.g., ASTM F1249 for plastic films). As used herein, a container with “impermeable to oxygen” is defined as having an oxygen transmission rate (OTR) of <0.1 cc/100 in²/24 hours at 23° C. and 90% relative humidity, as measured by an applicable standardized test method (e.g., ASTM D3985 for plastic films).

The terms “antimicrobial” or “anti-microbial” may be understood to refer to a substance or mixture of substances capable of killing or inhibiting the growth of a microbe, preventing the development of a microbe, or inhibiting the pathogenic action of a microbe. An antimicrobial substance can act through a physical or chemical mechanism.

The terms “antibiofilm” or “anti-biofilm” may be understood to refer to a substance or mixture of substances capable of killing or inhibiting the growth of a microbial biofilm, preventing the development of a microbial biofilm, or inhibiting the pathogenic action of a microbial biofilm. An anti-biofilm substance can act through a physical or chemical mechanism.

The term “disinfectant” may be understood to refer to a substance or mixture of substances that destroys or irreversibly inactivates bacteria, fungi, or viruses, but not necessarily bacterial spores, in the inanimate environment. A disinfectant may have anti-biofilm activity and can act through a physical or chemical mechanism.

The term “minimum biofilm eradicated concentration” or “MBEC” may be understood to refer to the minimal antimicrobial concentration required to kill a biofilm. MBEC can be determined qualitatively or quantitatively.

The terms “minimum biofilm inhibitory concentration” or “MBIC” may be understood to refer to the minimal antimicrobial concentration at which there is no time-dependent increase in viable microbial cells of a biofilm. MBIC can be determined qualitatively or quantitatively.

The term “contact time” may be understood to refer to an amount of time that a biofilm is exposed to a test substance.

The term “assay” may be understood to mean an investigative procedure for qualitatively assessing or quantitatively measuring the presence, amount, or functional activity of a target entity.

The term “substance” may be understood to mean a material, matter or that which has definite chemical composition and distinct properties, including both pure substances and mixtures.

The term “test substance” may be understood to refer to a substance being evaluated for antimicrobial activity. A test substance can be formulated in a variety of ways, for example, as a liquid, a solid, a wipe, a spray, a gel, a paste, or a powder. A test substance can act by a chemical or physical mechanism.

The term “array” may be understood to refer to an ordered arrangement. The term “dehydrated biofilm array” may be understood to refer to an ordered arrangement of dehydrated biofilm compositions. The term “N×N array” may be understood to refer to an ordered arrangement defined by an N×N pattern where N is an integer. The term “N1×N2 array” may be understood to refer to an ordered arrangement defined by an N1×N2 pattern where N1 and N2 are integers, and where the integers N1 and N2 may be the same integer or may be a different integer. The term “N1×N2 array” can be used interchangeably with the term “N×N array”.

Unless otherwise defined, all technical and scientific terms used herein have the same meaning as commonly understood by one of ordinary skill in the art to which this disclosure belongs. Methods and materials are described herein for use in the present disclosure; other, suitable methods and materials can also be used. The materials, methods, and examples are illustrative only and not intended to be limiting.

All publications, patent applications, patents, sequences, database entries, journal articles, and other references mentioned herein are expressly incorporated by reference in their entirety.

Biofilms may be understood to be complex aggregates of microorganisms enmeshed in a self-produced extracellular polymeric matrix. The microorganisms in a biofilm may exhibit a multicellular lifestyle and group behavior. Biofilms can have significant negative economic, health, and safety impacts. According to National Institutes of Health (NIH) more than 80% of all bacterial infections are associated with biofilms (Akkers et al., BMC Infect. Dis. 14:190, 2014). The CDC estimates that hospital acquired infections have annual direct costs for US hospitals of as much as $45 billion (The Centers for Disease Control and Prevention, 2009). Biofilms may also contribute to foodborne illness. The CDC estimates that each year in the US 76 million people are infected with a foodborne illness, resulting in 325,000 hospitalizations and 5,000 deaths (Nyachuba, Nutr. Rev. 68:257-269, 2010). Additionally, biofilm-associated corrosion and biofouling leads to significant damage and costs to infrastructure, manufacturing, oil, gas, and military industries.

The complex interactions of microorganisms in a biofilm and the components of the extracellular polymeric matrix collectively produce the biofilm phenotype. Biofilms are difficult to culture in a laboratory setting with substantial repeatability and reproducibility, and often require extensive expertise, equipment, and other resources to culture. Small differences in environmental conditions and methodology can lead to significant variability in biofilm cultures. The multifaceted properties and complex characteristics of biofilms hinder researchers' ability to reliably and reproducibly study anti-biofilm activity. This causes significant difficulties in interpreting and comparing data from anti-biofilm testing.

It is accordingly an object of the present disclosure to provide dehydrated biofilm compositions and methods for preparing said compositions that can significantly reduce the complexities and difficulties in studying anti-biofilm activity, and also provide for extensive stability and preservation of biofilm viability, integrity, and function upon rehydration. Still further objects and advantages will become apparent from consideration of the ensuing description and drawings.

This disclosure is based, at least in part, on the discovery that a biofilm can be stably dehydrated and subsequently rehydrated without destroying the biofilm phenotype, and without causing substrate detachment and irreversibly breaking down the three dimensional architecture, structure, and function of a biofilm. Biofilms are highly hydrated multicellular communities, comprising as much as 97% water. The high water content of a biofilm's extracellular matrix is integral to its three dimensional architecture, structure, function, and viability. Surprisingly, it was discovered that the dehydrated biofilm compositions provided herein can be prepared so that they are able to be subsequently rehydrated while retaining biofilm integrity, substrate attachment, and viability. It was further discovered that the dehydrated biofilm compositions provided herein have unexpected stability and preservation of biofilm phenotype properties so that they can be stored for an extensive period of time and utilized, e.g., in determining the anti-biofilm efficacy of a test substance. In view of this discovery, provided herein are dehydrated biofilm compositions comprising a microbial cell population enmeshed in a self-produced extracellular polymeric matrix and attached to a substrate, wherein the composition retains substrate attachment and exhibits a viable cell count and a biofilm phenotype upon rehydration.

In some embodiments, the composition comprises a viable cell count between about 1×10³ CFU and about 1×10¹² CFU per composition or per cm² upon rehydration. In some embodiments, the composition comprises a viable cell count between about 1×10⁵ CFU and about 1×10¹⁰ CFU per composition or per cm² upon rehydration.

In some embodiments, the composition further comprises a stabilizing agent. In some examples, the stabilizing agent acts as a lyoprotectant, a cryoprotectant, a buffering agent, an anti-oxidizing agent, and/or a bulking agent. In some examples, the composition comprises a stabilizing agent that is selected from the group consisting of: a sugar, a polyol, a polymer, an antioxidant, an amino acid, a surfactant, and a buffer. In some examples, the composition comprises between about 10 mg to about 200 mg of a sugar and between about 5 mg to about 60 mg of an antioxidant per composition or per cm². In some examples, the composition further comprises between about 0.1 mg to about 5 mg of a buffer per composition or per cm².

In some embodiments, the composition comprises a residual water content of between about 0% to about 15% by weight of the composition. In some examples, the composition comprises a residual water content of between about 0% to about 10% by weight of the composition. In some examples, the composition comprises a residual water content of between about 0% to about 7.5% by weight of the composition. In some embodiments, the composition exhibits a water activity of less than about 0.9 Aw. In some examples, the composition exhibits a water activity of less than about 0.6 Aw. In some examples, the composition exhibits a water activity of less than about 0.3 Aw.

In some embodiments, the composition has a loss of viability of less than about 3 log CFU per composition or per cm² over a period of about 30 days at a temperature of about 5° C. In some examples, the composition has a loss of viability of less than about 3 log CFU per composition or per cm² over a period of about 60 days at a temperature of about 5° C. In some examples, the composition has a loss of viability of less than about 3 log CFU per composition or per cm² over a period of about 90 days at a temperature of about 5° C.

In some embodiments, the composition comprises an organism selected from the group consisting of: Acinetobacter spp., Aspergillus spp., Bacillus spp., Bordetella spp., Burkholderia spp., Campylobacter spp., Candida spp., Clostridium spp., Corynebacterium spp., Cronobacter spp., Enterobacter spp., Enterococcus spp., Escherichia spp., Haemophilus spp., Klebsiella spp., Legionella spp., Listeria spp., Mycobacterium spp., Peniciullium spp., Proteus spp., Pseudomonas spp., Salmonella spp., Serratia spp., Shigella spp., Staphylococcus spp., Streptococcus spp., Trichophyton spp., Vibrio spp., or Yersinia spp.

In some examples, the composition comprises an organism selected from the group consisting of: Acinetobacter baumannii, Aspergillus brasiliensis, Aspergillus niger, Bacillus cereus, Bacillus subtilis, Bordetella pertussis, Burkholderia cepacia, Campylobacter jejuni, Candida albicans, Candida auris, Clostridium difficile, Clostridium sporogenes, Clostridium botulinum, Corynebacterium ammoniagenes, Cronobacter sakazakii, Enterobacter aerogenes, Enterobacter cloacae, Enterococcus faecalis, Escherichia coli, Haemophilus influenzae, Klebsiella oxytoca, Klebsiella pneumoniae, Legionella pneumophila, Listeria monocytogenes, Mycobacterium bovis, Mycobacterium terrae, Peniciullium chrysogenum, Proteus mirabilis, Pseudomonas aeruginosa, Salmonella bongori, Salmonella enterica, Serratia marcescens, Shigella dysenteriae, Shigella flexneri, Shigella sonnei, Staphylococcus aureus, Staphylococcus epidermidis, Streptococcus pneumoniae, Streptococcus pyogenes, Trichophyton mentagrophytes, Vibrio cholera, Yersinia enterocolitica, or a strain thereof.

In some examples, the composition comprises an organism selected from the group consisting of: Acinetobacter baumannii (ATCC 19606), Aspergillus brasiliensis (ATCC 16404), Aspergillus niger (ATCC 6275), Aspergillus niger (ATCC 16404), Bordetella pertussis (ATCC 12743), Campylobacter jejuni (ATCC 33291), Campylobacter jejuni (ATCC 29428), Candida albicans (ATCC 10231), Clostridium difficile (ATCC 43598), Cronobacter sakazakii (ATCC 12868), Enterobacter aerogenes (ATCC 13048), Enterococcus faecalis (ATCC 29212), Escherichia coli (ATCC 10536), Escherichia coli (ATCC 11229), Escherichia coli O157:H7 (ATCC 35150), Haemophilus influenzae (ATCC 10211), Klebsiella oxytoca (ATCC 13182), Klebsiella pneumoniae (ATCC 4352), Legionella pneumophila (ATCC 33153), Listeria monocytogenes (ATCC 19117), Listeria monocytogenes (ATCC 19111), Listeria monocytogenes (ATCC 7644), Methicillin Resistant Staphylococcus aureus (ATCC BAA-1683), Methicillin Resistant Staphylococcus aureus (ATCC 33592), Multi-drug Resistant Enterococcus faecium (ATCC 51559), Multi-drug resistant Klebsiella pneumoniae (ATCC 51503), Mycobacterium bovis (ATCC 35743), Mycobacterium terrae (ATCC 15755), Proteus mirabilis (ATCC 9240), Pseudomonas aeruginosa (ATCC 15442), Salmonella enterica (ATCC 10708), Salmonella enterica (ATCC 6539), Serratia marcescens (ATCC 14756), Shigella dysenteriae (ATCC 11835), Shigella flexneri (ATCC 29508), Shigella sonnei (ATCC 11060), Staphylococcus aureus (ATCC 6538), Staphylococcus epidermidis (ATCC 12228), Streptococcus pneumoniae (ATCC 6305), Streptococcus pyogenes (ATCC 12384), Streptococcus pyogenes (ATCC 19615), Trichophyton interdigitale (ATCC 9533), Trichophyton mentagrophytes (ATCC 9533), Vancomycin Resistant Enterococcus faecalis (ATCC 51575), Vibrio cholera (ATCC 11623), or Yersinia enterocolitica (ATCC 23715).

In some embodiments, the composition is enclosed within a primary container. In some examples, the primary container has a low permeability to moisture and oxygen. In some examples, the primary container is sealed within a secondary container that has low permeability to moisture and oxygen. In some examples, the primary container or secondary container is impermeable to moisture and oxygen. In some examples, a desiccant is included within the secondary container. In some examples, an oxygen scavenger is included within the secondary container.

Also provided herein are methods of preparing a dehydrated biofilm composition (e.g., any of the dehydrated biofilm compositions described herein). In some embodiments, the dehydrated biofilm composition is dehydrated by lyophilization. In some embodiments, the dehydrated biofilm composition is prepared by a process that includes: (1) growing a biofilm composition on a substrate, (2) adding a stabilizing agent to the biofilm composition, (3) freezing the biofilm composition, (4) dehydrating the biofilm composition by sublimation, (5) dehydrating the biofilm composition by desorption, and (6) enclosing the dehydrated biofilm composition in a primary container. Also provided herein are methods of rehydrating a dehydrated biofilm composition (e.g., any of the dehydrated biofilm compositions provided herein) that comprise submerging the dehydrated biofilm composition in an aqueous solution and maintaining the dehydrated biofilm composition in the aqueous solution for an amount of time sufficient for reconstitution.

In some embodiments, the composition is used to evaluate the anti-biofilm efficacy of a test substance. In some examples, the anti-biofilm efficacy of the test substance is evaluated by a process that includes: (1) submerging a dehydrated biofilm composition in an aqueous solution, (2) maintaining the composition in the aqueous solution for an amount of time sufficient for rehydration, (3) contacting the rehydrated biofilm composition with a test substance for a contact time, (4) performing an assay to determine the anti-biofilm efficacy of the test substance. In some examples, evaluating the anti-biofilm efficacy of the test substance includes evaluating the ability of a test substance to remove a biofilm from a substrate or the ability to kill microbes in a biofilm state.

Also provided herein are dehydrated biofilm arrays comprising a plurality of reservoir compartments arranged into an N×N array, wherein one or more reservoirs contains a dehydrated biofilm composition comprising a viable microbial cell population enmeshed in a self-produced extracellular matrix, wherein the dehydrated biofilm composition retains substrate attachment and exhibits a viable cell count and a biofilm phenotype upon rehydration. Also provided herein are methods of preparing and dosing a dehydrated biofilm array (e.g., any of the dehydrated biofilm arrays provided herein) that include providing a plurality of biofilm compositions each comprising a microbial cell population enmeshed in a self-produced extracellular polymeric matrix, containing each of the biofilm compositions in reservoir compartments organized into a N1×N2 array, coating or submerging each biofilm composition in an effective amount of a stabilizing agent, freezing each biofilm composition, subjecting each biofilm composition to a primary drying phase (e.g., dehydration by sublimation), subjecting each biofilm composition to a secondary drying phase (e.g., dehydration by desorption), rehydrating each biofilm composition in the N1×N2 array, and contacting the array with one or more substances (e.g., a compound).

Also provided are kits including any of the dehydrated biofilm compositions described herein. Also provided herein are kits for evaluating the anti-biofilm efficacy of a test substance comprising: a dehydrated biofilm composition comprising a microbial cell population enmeshed in a self-produced extracellular polymeric matrix and attached to a substrate, wherein the composition retains substrate attachment and exhibits a viable cell count and a biofilm phenotype upon rehydration, wherein the dehydrated biofilm composition comprises a viable cell count between about 1×10³ CFU and about 1×10¹² CFU upon rehydration, wherein the dehydrated biofilm composition comprises a stabilizing agent, wherein the dehydrated biofilm composition comprises a residual water content of between about 0% to about 10% by weight of the composition, wherein the dehydrated biofilm composition has a loss of viability of less than 3 log CFU over a period of about 90 days at a temperature of about 5° C., wherein the dehydrated biofilm composition is enclosed within a primary container, and wherein the primary container is sealed within a secondary container that has low permeability to moisture and oxygen. In some examples, the kit comprises a plurality of reservoir compartments arranged into an N×N array, wherein one or more reservoirs contains a dehydrated biofilm composition.

Provided herein are dehydrated biofilm compositions comprising a microbial cell population enmeshed in a self-produced extracellular polymeric matrix wherein upon rehydration the microbial cell population has a viable cell count and a biofilm phenotype. Also provided herein are arrays comprising a plurality of dehydrated biofilm compositions wherein each dehydrated biofilm composition is characterized by a microbial cell population enmeshed in a self-produced extracellular polymeric matrix and wherein upon rehydration the microbial cell population has a viable cell count and a biofilm phenotype.

The dehydrated biofilm compositions, arrays, and methods provided herein allow for the stable preservation and subsequent rehydration of dehydrated biofilms with retention of biofilm characteristics and behavior. For example, the compositions, arrays, and methods provided herein allow for the rehydration of a dehydrated biofilm composition wherein the rehydrated biofilm composition contains a viable cell count of at least 1×10³ colony forming units (CFU) (e.g., at least 1×10³ CFU, at least 1×10⁴ CFU, at least 1×10⁵ CFU, or at least 1×10⁶ CFU) enmeshed in a self-produced extracellular polymeric matrix (e.g., an organic polymer matrix comprising polysaccharides, extracellular DNA and proteins) upon rehydration. In any of the compositions or methods described herein the dehydrated biofilm composition can be stable for at least 1 month (e.g., at least 2 months, at least 2.5 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, at least 24 months, at least 27 months, at least 30 months, at least 36 months, at least 48 months, or at least 60 months). Upon rehydration, dehydrated biofilm compositions and arrays included herein exhibit phenotypic and genotypic properties of biofilms (e.g., 3-dimensional structure, profiles of gene and protein expression, metabolism, antimicrobial resistance and tolerance characteristics of a biofilm state as compared to a planktonic state).

As can be appreciated in the art, the various aspects described below can be used in combination without limitation.

Dehydrated Biofilm Compositions

Provided herein are dehydrated biofilm compositions that comprise a microbial cell population enmeshed in a self-produced extracellular polymeric matrix (e.g., a natural, microbe-produced polysaccharide matrix) where the composition has a viable cell count (e.g., between about 1×10³ CFU to about 1×10⁹ CFU) and biofilm phenotype (e.g., a substrate attached microbial community enmeshed in a self-produced extracellular matrix) upon rehydration. Methods for preparing any of the dehydrated biofilm compositions provided herein are also described below. Methods for rehydrating any of the dehydrated biofilm compositions provided herein are also described below. Dehydrated biofilm arrays comprising any of the dehydrated biofilm compositions provided herein are described below. Methods for preparing and dosing dehydrated biofilm arrays are also described below.

Non-limiting examples of microbial cell populations that can be included in any of the dehydrated biofilm compositions provided herein are described below. Non-limiting examples of microbial organisms (e.g., any of the exemplary microbial organisms described herein) that can be included in any of the dehydrated biofilm compositions provided herein are described below. Non-limiting examples of the number of different microbial organisms that can be included in any of the dehydrated biofilm compositions provided herein are also described below. Non-limiting examples of the types of microbial organisms that can be included in any of the dehydrated biofilm compositions provided herein are also described below. Non-limiting examples of the sources of microbial organisms that can be included in any of the dehydrated biofilm compositions provided herein are also described below. Methods for culturing the microbial cell populations provided herein are described below. Some examples of any of the dehydrated biofilm compositions described herein can be attached to a substrate (e.g., any of the exemplary substrates described herein). Non-limiting examples of substrates that can be included in any of the dehydrated biofilm compositions provided herein are described below.

Any of the dehydrated biofilm compositions provided herein can have a viable cell count of at least 1×10² CFU (e.g., at least 1×10² CFU, at least 2×10² CFU, at least 3×10² CFU, at least 4×10² CFU, at least 5×10² CFU, at least 6×10² CFU, at least 7×10² CFU, at least 8×10² CFU, at least 9×10² CFU, at least 1×10³ CFU, at least 2×10³ CFU, at least 3×10³ CFU, at least 4×10³ CFU, at least 5×10³ CFU, at least 6×10³ CFU, at least 7×10³ CFU, at least 8×10³ CFU, at least 9×10³ CFU, at least 1×10⁴ CFU, at least 2×10⁴ CFU, at least 3×10⁴ CFU, at least 4×10⁴ CFU, at least 5×10⁴ CFU, at least 6×10⁴ CFU, at least 7×10⁴ CFU, at least 8×10⁴ CFU, at least 9×10⁴ CFU, at least 1×10⁵ CFU, at least 2×10⁵ CFU, at least 3×10⁵ CFU, at least 4×10⁵ CFU, at least 5×10⁵ CFU, at least 6×10⁵ CFU, at least 7×10⁵ CFU, at least 8×10⁵ CFU, at least 9×10⁵ CFU, at least 1×10⁶ CFU, at least 2×10⁶ CFU, at least 3×10⁶ CFU, at least 4×10⁶ CFU, at least 5×10⁶ CFU, at least 6×10⁶ CFU, at least 7×10⁶ CFU, at least 8×10⁶ CFU, at least 9×10⁶ CFU, at least 1×10⁷ CFU, at least 2×10⁷ CFU, at least 3×10⁷ CFU, at least 4×10⁷ CFU, at least 5×10⁷ CFU, at least 6×10⁷ CFU, at least 7×10⁷ CFU, at least 8×10⁷ CFU, at least 9×10⁷ CFU, at least 1×10⁸ CFU, at least 2×10⁸ CFU, at least 3×10⁸ CFU, at least 4×10⁸ CFU, at least 5×10⁸ CFU, at least 6×10⁸ CFU, at least 7×10⁸ CFU, at least 8×10⁸ CFU, at least 9×10⁸ CFU, at least 1×10⁹ CFU, at least 2×10⁹ CFU, at least 3×10⁹ CFU, at least 4×10⁹ CFU, at least 5×10⁹ CFU, at least 6×10⁹ CFU, at least 7×10⁹ CFU, at least 8×10⁹ CFU, at least 9×10⁹ CFU, at least 1×10¹⁰ CFU, at least 2×10¹⁰ CFU, at least 3×10¹⁰ CFU, at least 4×10¹⁰ CFU, at least 5×10¹⁰ CFU, at least 6×10¹⁰ CFU, at least 7×10¹⁰ CFU, at least 8×10¹⁰ CFU, at least 9×10¹⁰ CFU, at least 1×10¹¹ CFU, at least 2×10¹¹ CFU, at least 3×10¹¹ CFU, at least 4×10¹¹ CFU, at least 5×10¹¹ CFU, at least 6×10¹¹ CFU, at least 7×10¹¹ CFU, at least 8×10¹¹ CFU, at least 9×10¹¹ CFU, at least 1×10¹² CFU, at least 2×10¹² CFU, at least 3×10¹² CFU, at least 4×10¹² CFU, at least 5×10¹² CFU, at least 6×10¹² CFU, at least 7×10¹² CFU, at least 8×10¹² CFU, at least 9×10¹² CFU, at least 1×10¹³ CFU, at least 2×10¹³ CFU, at least 3×10¹³ CFU, at least 4×10¹³ CFU, at least 5×10¹³ CFU, at least 6×10¹³ CFU, at least 7×10¹³ CFU, at least 8×10¹³ CFU, at least 9×10¹³ CFU, at least 1×10¹⁴ CFU, at least 2×10¹⁴ CFU, at least 3×10¹⁴ CFU, at least 4×10¹⁴ CFU, at least 5×10¹⁴ CFU, at least 6×10¹⁴ CFU, at least 7×10¹⁴ CFU, at least 8×10¹⁴ CFU, or at least 9×10¹⁴ CFU) per composition or per cm² upon rehydration.

Any of the dehydrated biofilm compositions provided herein can have a viable cell count of between about 1×10² CFU and about 1×10¹⁴ CFU (e.g., a viable cell count of between about 1×10² CFU and about 1×10⁸ CFU, a viable cell count of between about 1×10² CFU and about 1×10⁶ CFU, a viable cell count of between about 1×10² CFU and about 1×10⁴ CFU, a viable cell count of between about 1×10⁴ CFU and about 1×10⁸ CFU, a viable cell count of between about 1×10³ CFU and about 1×10⁹ CFU, a viable cell count of between about 1×10⁵ CFU and about 1×10⁹ CFU, a viable cell count of between about 1×10⁵ CFU and about 1×10⁸ CFU, a viable cell count of between about 1×10⁶ CFU and about 1×10⁹ CFU, a viable cell count of between about 1×10⁶ CFU and about 1×10⁸ CFU, a viable cell count of between about 1×10⁷ CFU and about 1×10⁸ CFU, or a viable cell count of between about 1×10⁶ CFU and about 1×10⁷ CFU) per composition or per cm² upon rehydration. In some examples of the dehydrated biofilm compositions provided herein, the composition has a viable cell count of between about 1×10⁵ CFU and about 1×10¹⁰ CFU (e.g., a viable cell count of between about 1×10⁵ CFU and about 1×10⁹ CFU, a viable cell count of between about 1×10⁵ CFU and about 1×10⁸ CFU, a viable cell count of between about 1×10⁵ CFU and about 1×10⁷ CFU, a viable cell count of between about 1×10⁵ CFU and about 1×10⁶ CFU, a viable cell count of between about 1×10⁶ CFU and about 1×10¹⁰ CFU, a viable cell count of between about 1×10⁶ CFU and about 1×10⁹ CFU, a viable cell count of between about 1×10⁶ CFU and about 1×10⁸ CFU, a viable cell count of between about 1×10⁶ CFU and about 1×10⁷ CFU, a viable cell count of between about 1×10⁷ CFU and about 1×10¹⁰ CFU, a viable cell count of between about 1×10⁷ CFU and about 1×10⁹ CFU, a viable cell count of between about 1×10⁷ CFU and about 1×10⁸ CFU, a viable cell count of between about 1×10⁸ CFU and about 1×10¹⁰ CFU, a viable cell count of between about 1×10⁸ CFU and about 1×10⁹ CFU, or a viable cell count of between about 1×10⁹ CFU and about 1×10¹⁰ CFU) per composition or per cm² upon rehydration. The viable cell count of any of the dehydrated biofilm compositions provided herein can be determined using a suitable method, e.g., manual cell counting (e.g., using plating and colony forming unit (CFU) enumeration) or automated cell counting (e.g., flow-based cell counting), see e.g., Wilson et al., Res. Rev. J. Eng. Technol. 6, 2017.

Some examples of any of the dehydrated biofilm compositions described herein can further include a stabilizing agent (e.g., any of the exemplary stabilizing agents described herein). In some examples, the dehydrated biofilm composition includes one stabilizing agent. In other examples, the dehydrated biofilm composition includes more than one (e.g., two, three, four, five, six, seven, eight, nine, or ten) stabilizing agents. Non-limiting examples of stabilizing agents that can be included in any of the dehydrated biofilm compositions provided herein include: a sugar (e.g., a monosaccharide, a disaccharide, a reducing sugar, or a non-reducing sugar), a polyol, a polymer (e.g., an oligosaccharide, a polysaccharide, a cellulose-derivative, or a synthetic polymer), an antioxidant, an amino acid, a surfactant, and a buffer. When included in any of the compositions herein, a stabilizing agent can act as a lyoprotectant, a cryoprotectant, a buffering agent, an antioxidant, or a bulking agent. Surfactants can be present in any of the compositions of the disclosure, e.g., to stabilize and/or enhance the solubility of other constituents. Buffers can be included in any of the compositions of the disclosure, e.g., to stabilize other constituents and/or control pH. Cryoprotectants, when included in any of biofilm compositions described herein prior to the the freezing phase of the dehydration process, can protect the biofilm composition from damage during the freezing process (e.g., damage caused by ice formation impacting viability of biological components in any of the biofilm compositions described herein). The cryoprotectant is added to the biofilm composition prior to freezing in an amount adequate to protect the composition during the freezing process and improve its viability following freezing. Cryoprotectants can also be added as a bulking agent in any of the dehydrated biofilm compositions described herein. Lyoprotectants, when included in any of biofilm compositions described herein prior to dehydration, can improve the long term stability of the composition. Lyoprotectants can protect the microbial cell population and extracellular matrix during the dehydration process and in subsequent storage. The lyoprotectant is added to the biofilm composition prior to dehydration in an amount adequate to protect the composition during the dehydration process and improve its stability in subsequent storage. Lyoprotectants can also be added as a bulking agent in any of the dehydrated biofilm compositions described herein. The cryoprotectant or lyoprotectant excipients are added to the biofilm composition prior to dehydration in suitable amounts such that the physical and chemical stability and integrity of the biofilm are retained upon dehydration and subsequent rehydration. In some embodiments, no cryoprotectant or lyoprotectant excipients are added. In other embodiments, a lyoprotectant is added but no cryoprotectant is added. Non-limiting examples of stabilizing agent(s) that can be included in any of the dehydrated biofilm compositions are described below. Methods for preparing any of the dehydrated biofilm compositions provided herein using a stabilizing agent are described below.

Any of the dehydrated biofilm compositions provided herein can comprise a residual water content (e.g., a measurable amount of water) or a residual moisture content. For example, in some of the embodiments of the compositions described herein the dehydrated biofilm composition can comprise between about 0% to about 25% residual water content or residual moisture content. In some of the embodiments of the compositions described herein the dehydrated biofilm composition can comprise between about 0% to about 15% water (e.g., between about 0% water and about 5% water, between 0.5% water and about 5% water, between about 1% water and about 5% water, between about 2% water and about 4% water, between about 1% water and about 4% water, between about 1% water and about 3% water, between about 1% water and about 2% water, between about 0.25% water and about 1% water, between about 0.25% water and about 0.75% water, or between about 0% water and about 0.05% water) by weight of the composition (e.g., w/w). Additional non-limiting examples of the residual water content or the residual moisture content that can be present in any of the compositions provided herein are further described below.

In some examples, the dehydrated biofilm composition is contained within a reservoir or reservoir compartment. In some examples, the reservoir or reservoir compartment can have a volume of between about 1 uL and about 50 mL (e.g., between about 100 uL and about 500 uL, between about 750 uL and about 1.25 mL, between about 4 mL and about 6 mL, between about 9 mL and about 11 mL, between about 14 mL and about 16 mL, between about 19 mL and about 21 mL, between about 24 mL and about 26 mL, between about 29 mL and about 31 mL, between about 34 mL and about 36 mL, between about 39 mL and about 41 mL, between about 44 mL and about 46 mL, or between about 45 mL and about 50 mL). Additional non-limiting examples of the volume of the reservoir or reservoir compartment are further described below. Non-limiting examples of primary containers include a well of a multiple well plate (e.g., a 6 well plate, a 12 well plate, a 24 well plate, a 48 well plate, a 96 well plate, a 384 well plate, a 1536 well plate), a test tube (e.g., a culture tube, a centrifuge tube, a microcentrifuge tube, a PCR tube, a conical tube, or a freestanding tube), or a vial (e.g., a screw-thread vial, a snap-cap vial, or a crimp-top vial)). Additional examples of primary containers are contemplated.

The storage conditions of at least some of the dehydrated biofilm compositions described herein can impact long-term viability and stability. It may be desirable minimize any moisture or oxygen from interacting with the dehydrated biofilm composition. In some embodiments, a dehydrated biofilm composition is sealed in an airtight container to prevent atmospheric exposure. Methods for sealing containers to prevent atmospheric exposure may include those disclosed in U.S. Pat. No. 8,544,665B2. In some embodiments, a dehydrated biofilm composition is sealed in a container by stoppering, e.g., under partial vacuum. In some embodiments, the container is backfilled, e.g., with an inert gas.

In some instances, a dehydrated biofilm composition is enclosed within a primary container and the primary container is sealed within a secondary container (e.g., that has low permeability to moisture). In some embodiments, more than one biofilm composition is enclosed in a single primary container. In some examples, more than one primary containers containing a dehydrated biofilm composition are sealed within a secondary container. In at least some instances, the primary container and/or secondary container has a minimal moisture vapor transmission rate and oxygen transfer rate. In some instances, the primary container and/or secondary container is impermeable to oxygen transfer and moisture transmission. Suitable methods can be used to measure moisture vapor transmission rate (see, e.g., ASTM F1249 and Federal Test Method Standard 101 Method 3030) (see, e.g., ASTM D3985, ASTM F3136, and ASTM F1307) and oxygen transmission rate. In some examples, the container (e.g., the primary container and/or secondary container) is a moisture barrier bag or a foil bag. In some examples, the container (e.g., the primary container and/or secondary container) is impenetrable by microorganisms and does not allow microorganisms to transfer in or out of the container. In some examples, the primary container is a microtiter plate and the secondary container is a Mylar® bag that has low permeability to moisture vapor and oxygen.

In some examples, the secondary container can include a desiccant and/or an oxygen scavenger, e.g., in order to maintain a dry atmosphere and reduce any moisture surrounding the primary container. Non-limiting examples of desiccant include silica, activated charcoal, calcium sulfate, calcium chloride, and/or a molecular sieve (e.g., zeolite). Additional desiccants may be utilized. Non-limiting examples of oxygen scavengers include mixtures of iron powder and sodium chloride. Additional oxygen scavengers may be utilized.

Temperature can impact the stability of any of the dehydrated biofilm compositions described herein. In some embodiments, the dehydrated biofilm composition can be stored at a room temperature (e.g., between about 20° C. and about 25° C.). In other embodiments, the dehydrated biofilm composition can be stored at a refrigerated temperature (e.g., between about 2° C. and about 7° C.). Light (e.g., ultraviolet light) exposure can also impact the stability of any of the dehydrated biofilm compositions described herein. In some embodiments, the composition is stored in a dark room. In other embodiments, the composition can be stored in a container that is tinted, covered, or otherwise intended to reduce light exposure. Additional methods for reducing exposure to moisture, temperature, and light during storage are contemplated.

Any of the dehydrated biofilm compositions provided herein can be used as part of a bioassay (e.g., a cell-based screening assay). In some examples, any of the dehydrated biofilm compositions provided herein can be used to evaluate the ability of a test substance to kill the organisms in a biofilm or to remove an attached biofilm from a substrate. For example, any of the dehydrated biofilm compositions described herein can be used to evaluate the anti-biofilm activity of a test substance. In some examples, any of the dehydrated biofilm compositions provided herein can be used to evaluate the ability of a test substance to modify, e.g., degrade or disrupt, the extracellular polymeric matrix of a biofilm. In some examples, any of the dehydrated biofilm compositions provided herein can be used to evaluate the ability of a test substance or an organism to modify the microbial cell population and its composition within a biofilm, e.g., to decrease or increase the presence of a particular organism within the microbial cell population of a biofilm. In some examples, any of the dehydrated biofilm compositions provided herein can be used to evaluate the ability of a test substance to modify the metabolite production (e.g., increase or decrease the production of a particular metabolite or series of metabolites), protein expression (e.g., increase or decrease the expression of a particular protein or series of proteins), gene expression (e.g., increase or decrease the expression of a particular gene or series of genes), architecture, composition, or other characteristic or behavior of a biofilm. In some examples, any of the dehydrated biofilm compositions provided herein can be used to determine the minimum biofilm eradication concentration (MBEC) or minimum biofilm inhibitory concentration (MBIC) of a test substance. In some examples, any of the dehydrated biofilm compositions provided herein can be used in quality control activities, e.g., to detect the presence of a pollutant, to assess the efficacy of cleaning, disinfecting, or sterilizing activities, or to determine proof-of-efficacy (e.g., in disinfectant lot release testing). As can be appreciated by those skilled in the art, any of the dehydrated biofilm compositions provided herein can be used for other bioassays.

Microbial Cell Populations

In any of the dehydrated biofilm compositions provided herein a microbial cell population is a group of microorganisms (e.g., a cluster of microbial cells). In any of the dehydrated biofilm compositions provided herein, the microbe can be a prokaryotic or a eukaryotic organism. Non-limiting examples of the types of microorganisms that can be included in any of the dehydrated biofilm compositions provided herein include: bacteria, fungi, algae, protist, diatom, archaea, and cyanobacteria. In some embodiments, the microbial cell population in any of the dehydrated biofilm compositions provided herein can comprise a single type of microbial cell, at least two types of microbial cells, at least three types of microbial cells, at least four types of microbial cells, at least five types of microbial cells, or more than five types of microbial cells. Other methods for identifying the types of microbial organisms in a microbial cell population may be utilized.

In some embodiments of the dehydrated biofilm compositions provided herein, the microbial cell population can comprise a homogenous population of identical cells. In some embodiments, the microbial cell population can comprise a heterogeneous population of microbial cells. In some embodiments, the microbial cell population in any of the dehydrated biofilm compositions provided herein can comprise one organism, at least two different organisms, at least three different organisms, at least four different organisms, at least five different organisms, at least six different organisms, at least seven different organisms, at least eight different organisms, at least nine different organisms, at least ten different organisms, at least twenty different organisms, at least thirty different organisms, or more than thirty different organisms. Other methods for identifying the number of organisms in a microbial cell population may be utilized.

In some embodiments, the microbial cell population can comprise one species of microbe, at least two species of microbe, at least three species of microbe, at least four species of microbe, at least five species of microbe, at least six species of microbe, at least seven species of microbe, at least eight species of microbe, at least nine species of microbe, at least ten species of microbe, or more than ten species of microbe. In some embodiments, the microbial cell population can comprise one microbial strain, at least two microbial strains, at least three microbial strains, at least four microbial strains, at least five microbial strains, at least six microbial strains, at least seven microbial strains, at least eight microbial strains, at least nine microbial strains, at least ten microbial strains, at least fifteen microbial strains, at least twenty microbial strains, at least twenty-five microbial strains, at least thirty microbial strains, at least thirty-five microbial strains, at least forty microbial strains, at least forty-five microbial strains, at least fifty microbial strains, or more than fifty microbial strains. In some examples, the microbe is a gram-positive bacteria or a gram-negative bacteria. In some examples, the microbe is a fungi (e.g., a yeast). In some embodiments, the microbe is Acetobacter spp., Acetonema spp., spp., Acinetobacter spp., Actinomyces spp., Agrobacterium spp., Alkalibacillus spp., Ammoniphilus spp., Amphibacillus spp., Anaerobacter spp., Anaerospora spp., Anaplasma spp., Aneurinibacillus spp., Anoxybacillus spp., Arthrobacter spp., Aspergillus spp., Aureobasidium spp., Azorhizobium spp., Azotobacter spp., Bacillus spp., Bacteroides spp., Bartonella spp., Beggiatoa spp., Bifidobacterium spp., Brevibacterium spp., Brevibacillus spp., Bordetella spp., Borrelia spp., Brucella spp., Burkholderia spp., Caldanaerobacter spp., Caloramator spp., Calymmatobacterium spp., Caminicella spp., Campylobacter spp., Candida spp., Cerasibacillus spp., Chlamydia spp., Chlamydophila spp., Cladosporium spp., Clostridium spp., Clostridiisalibacter spp., Cohnella spp., Corynebacterium spp., Coxiella spp., Cronobacter spp., Cryptococcus spp., Dendrosporobacter spp., Desulfotomaculum spp., Desulfosporomusa spp., Desulfosporosinus spp., Desulfovibrio spp., Desulfovirgula spp., Desulfunispora spp., Desulfurispora spp., Ehrlichia spp., Enterobacter spp., Enterococcus spp., Escherichia spp., Ferrobacillus spp., Filifactor spp., Filobacillus spp., Francisella spp., Fusarium spp., Fusobacterium spp., Gallionella spp., Gardnerella spp., Gelria spp., Geobacillus spp., Geosporobacter spp., Gracilibacillus spp., Haemophilus spp., Halobacillus spp., Halonatronum spp., Helicobacter spp., Heliobacterium spp., Heliophilum spp., Hormoconis spp., Klebsiella spp., Laceyella spp., Lactobacillus spp., Lactococcus spp., Legionella spp., Lentibacillus spp., Leptothrix spp., Listeria spp., Lysinibacillus spp., Megasphaera spp., Mahella spp., Metabacterium spp., Methanobacterium spp., Microbacterium spp., Micrococcus spp., Moorella spp., Moraxella spp., Mycobacterium spp., Mycoplasma spp., Natroniella spp., Neisseria spp., Oceanobacillus spp., Orenia spp., Ornithinibacillus spp., Oxalophagus spp., Oxobacter spp., Paenibacillus spp., Paraliobacillus spp., Pasteurella spp., Pediococcus spp., Pelospora spp., Pelotomaculum spp., Penicillium spp., Peptostreptococcus spp., Piscibacillus spp., Planifilum spp., Pluralibacter spp., Pneumocystis spp., Pontibacillus spp., Porphyromonas spp., Prevotella spp., Propionibacterium spp., Propionispora spp., Proteus spp., Pseudomonas spp., Ralstonia spp., Rhizobium spp., Rhodococcus spp., Rickettsia spp., Rochalimaea spp., Rothia spp., Saccharomyces spp., Salinibacillus spp., Salmonella spp., Salsuginibacillus spp., Seinonella spp., Serratia spp., Shewanella spp., Shigella spp., Shimazuella spp., Sinorhizobium spp., Spirillum spp., Sporacetigenium spp., Sporoanaerobacter spp., Sporobacter spp., Sporobacterium spp., Sporohalobacter spp., Sporolactobacillus spp., Sporomusa spp., Sporosarcina spp., Sporotalea spp., Sporotomaculum spp., Staphylococcus spp., Stenotrophomonas spp., Stomatococcus spp., Streptococcus spp., Syntrophomonas spp., Syntrophospora spp., Tenuibacillus spp., Tepidibacter spp., Terribacillus spp., Treponema spp., Thalassobacillus spp., Thermoacetogenium spp., Thermoactinomyces spp., The rmoalkalibacillus spp., Thermoanaerobacter spp., Thermoanaeromonas spp., Thermobacillus spp., Thermoflavimicrobium spp., Thermovenabulum spp., Thiobacillus spp., Thiothrix spp., Trichophyton spp., Trichosporon spp., Tuberibacillus spp., Vibrio spp., Virgibacillus spp., Viridans spp., Vulcanobacillus spp., Wolbachia spp., or Yersinia spp. In some embodiments, the microbe is Acetobacter aurantius, Acidithiobacillus thiooxidans, Acinetobacter baumannii, Actinomyces israelii, Agrobacterium radiobacter, Agrobacterium tumefaciens, Anaplasma phagocytophilum, Arthrobacter chlorophenolicus, Arthrobacter crystallopoietes, Arthrobacter luteus, Aspergillus brasiliensis, Aspergillus fumigatus, Aspergillus niger, Aureobasidium pullulans, Azorhizobium caulinodans, Azotobacter vinelandii, Bacillus anthracis, Bacillus atrophaeus, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bartonella henselae, Bartonella quintana, Beggiatoa alba, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Candida albicans, Candida auris, Candida dubliniensis, Candida krusei, Candida glabrata, Candida lusitaniae, Candida parapsilosis, Candida tropicalis, Chlamydia trachomatis, Chlamydophila pneumoniae, Chlamydophila psittaci, Cladosporium cladosporioides, Cladosporium resinae, Clostridium botulinum, Clostridium difficile, Clostridium perfringens, Clostridium sporogenes, Clostridium tetani, Corynebacterium ammoniagenes, Corynebacterium diphtheriae, Corynebacterium fusiforme, Corynebacterium glutamicum, Corynebacterium stationis, Coxiella burnetii, Cronobacter sakazakii, Cryptococcus neoformans, Desulfovibrio africanus, Desulfovibrio desulfuricans, Desulfovibrio salixigens, Desulfovibrio vulgaris, Desulfotomaculum orientis, Desulfotomaculum nigrificans, Ehrlichia chaffeensis, Enterobacter aerogenes, Enterobacter cloacae, Enterococcus avium, Enterococcus durans, Enterococcus faecalis, Enterococcus faecium, Enterococcus galllinarum, Enterococcus hirae, Enterococcus maloratus, Escherichia coli, Ferrobacillus ferrooxidans, Francisella tularensis, Fusobacterium nucleatum, Gallionella ferruginea, Gardnerella vaginalis, Geobacillus stearothermophilus, Haemophilus ducreyi, Haemophilus influenzae, Haemophilus parainfluenzae, Haemophilus pertussis, Haemophilus vaginalis, Helicobacter pylori, Hormoconis resinae, Klebsiella oxytoca, Klebsiella pneumoniae, Lactobacillus acidophilus, Lactobacillus bulgaricus, Lactobacillus casei, Lactobacillus plantarum, Lactococcus lactis, Legionella pneumophila, Leptothrix ochracea, Leptothrix discophora, Leptothrix cholodnii, Leptothrix lopholea, Leptothrix mobilis, Listeria monocytogenes, Methanobacterium extroquens, Microbacterium multiforme, Micrococcus luteus, Moraxella catarrhalis, Moraxella osloensis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium terrae, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Paenibacillus glucanolyticus, Pasteurella multocida, Pasteurella tularensis, Penicillium chrysogenum, Pluralibacter gergoviae, Pneumocystis carinii, Pneumocystis murina, Porphyromonas gingivalis, Prevotella melaninogenica, Proteus mirabilis, Proteus vulgaris, Pseudomonas aeruginosa, Pseudomonas fluorescens, Pseudomonas putida, Ralstonia pickettii, Rhizobium leguminosarum, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella bongori, Salmonella carrau, Salmonella concord, Salmonella enterica, Salmonella enteritidis, Salmonella infantis, Salmonella newport, Salmonella schwarzengrund, Salmonella typhi, Salmonella typhimurium, Serratia marcescens, Shewanella oneidensis, Shewanella putrefaciens, Shigella dysenteriae, Shigella flexneri, Shigella sonnei, Sinorhizobium meliloti, Spirillum volutans, Staphylococcus aureus, Staphylococcus epidermidis, Staphylococcus hominis, Stenotrophomonas maltophilia, Stomatococcus mucilaginous, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus gordonii, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Thiobacillus concretivorus, Thiobacillus thioparus, Treponema pallidum, Treponema denticola, Trichophyton interdigitale, Trichophyton mentagrophytes, Trichosporon asahii, Vibrio cholerae, Vibrio comma, Vibrio parahaemolyticus, Vibrio vulnificus, Viridans streptococci, Yersinia enterocolitica, Yersinia pestis, Yersinia pseudotuberculosis, or a strain (e.g., a drug-resistant strain) thereof. In some embodiments, the microbe is Acinetobacter baumannii (ATCC 19606), Aspergillus brasiliensis (ATCC 16404), Aspergillus niger (ATCC 6275), Aspergillus niger (ATCC 16404), Bordetella pertussis (ATCC 12743), Campylobacter jejuni (ATCC 33291), Campylobacter jejuni (ATCC 29428), Candida albicans (ATCC 10231), Clostridium difficile (ATCC 43598), Cronobacter sakazakii (ATCC 12868), Enterobacter aerogenes (ATCC 13048), Enterococcus faecalis (ATCC 29212), Escherichia coli (ATCC 10536), Escherichia coli (ATCC 11229), Escherichia coli O157:H7 (ATCC 35150), Haemophilus influenzae (ATCC 10211), Klebsiella oxytoca (ATCC 13182), Klebsiella pneumoniae (ATCC 4352), Legionella pneumophila (ATCC 33153), Listeria monocytogenes (ATCC 19117), Listeria monocytogenes (ATCC 19111), Listeria monocytogenes (ATCC 7644), Methicillin Resistant Staphylococcus aureus (ATCC BAA-1683), Methicillin Resistant Staphylococcus aureus (ATCC 33592), Multi-drug Resistant Enterococcus faecium (ATCC 51559), Multi-drug resistant Klebsiella pneumoniae (ATCC 51503), Mycobacterium bovis (ATCC 35743), Mycobacterium terrae (ATCC 15755), Proteus mirabilis (ATCC 9240), Pseudomonas aeruginosa (ATCC 15442), Salmonella enterica (ATCC 10708), Salmonella enterica (ATCC 6539), Serratia marcescens (ATCC 14756), Shigella dysenteriae (ATCC 11835), Shigella flexneri (ATCC 29508), Shigella sonnei (ATCC 11060), Staphylococcus aureus (ATCC 6538), Staphylococcus epidermidis (ATCC 12228), Streptococcus pneumoniae (ATCC 6305), Streptococcus pyogenes (ATCC 12384), Streptococcus pyogenes (ATCC 19615), Trichophyton interdigitale (ATCC 9533), Trichophyton mentagrophytes (ATCC 9533), Vancomycin Resistant Enterococcus faecalis (ATCC 51575), Vibrio cholera (ATCC 11623), or Yersinia enterocolitica (ATCC 23715). Additional non-limiting examples of microbes that can be included in any of the dehydrated biofilm compositions provided herein. Other methods for identifying the microbes (e.g., the genus, species, or strain of a microbe) in a microbial cell population may be utilized.

In some embodiments of any of the compositions or methods described herein, the microbe can be naturally occurring or genetically modified. Other methods of genetically modifying bacteria may be utilized Non-limiting examples of the sources of microbes in any of the dehydrated biofilm compositions provided herein include a laboratory collection and an environmental sample. In some embodiments, the microbe is sourced from a preserved collection of reference microorganisms (e.g., the ATCC Bacteriology Collection). In some embodiments, the microbe is isolated from an environmental sample (e.g., a clinical sample collected from a human subject (e.g., a wound of a human subject) or isolated from an environmental sample collected from a surface (e.g., a surface of an object in a hospital or a surface of an object in a manufacturing plant)). Other methods of collecting and isolating microbes may be utilized.

Any of the microbial cell populations described herein can be cultured as a biofilm, e.g., in a laboratory. Other methods for culturing microbes as a biofilm may be utilized, see, e.g., Azeredo et al., Critical Reviews in Microbiology 43:313-351. Depending on the organism and the purpose of its use, certain conditions are desired for culturing microbes as biofilms, including but not limited to certain vessels (e.g., a biofilm reactor), substrates (e.g., a stainless steel disc, a polypropylene rod, or a glass slide), nutrients, flow dynamics (e.g., turbulent flow or laminar flow), shear stress, temperature, and time duration. Additional methods and conditions for culturing microbes as biofilms may be utilized. The microbial cell populations described herein can be grown as a biofilm on a substrate.

When cultured as a biofilm, the microbial cell population can have a viable cell count of at least 1×10² CFU (e.g., at least 1×10³ CFU, at least 1×10⁴ CFU, at least 1×10⁵ CFU, at least 1×10⁶ CFU, at least 1×10⁷ CFU, at least 1×10⁸ CFU, at least 1×10⁹ CFU, at least 1×10¹⁰ CFU, at least 1×10¹¹ CFU, at least 1×10¹², at least 1×10¹³ CFU, or at least 1×10¹⁴ CFU) per composition or per cm² before dehydration. For example, the viable cell count of the microbial cell population present before dehydration can be between about 1×10² CFU and about 1×10¹⁴ CFU (e.g., a viable cell count of between about 1×10⁵ CFU and about 1×10¹⁰ CFU, a viable cell count of between about 1×10⁵ CFU and about 1×10⁹ CFU, a viable cell count of between about 1×10⁵ CFU and about 1×10⁸ CFU, a viable cell count of between about 1×10⁵ CFU and about 1×10⁷ CFU, a viable cell count of between about 1×10⁵ CFU and about 1×10⁶ CFU, a viable cell count of between about 1×10⁶ CFU and about 1×10¹⁰ CFU, a viable cell count of between about 1×10⁶ CFU and about 1×10⁹ CFU, a viable cell count of between about 1×10⁶ CFU and about 1×10⁸ CFU, a viable cell count of between about 1×10⁶ CFU and about 1×10⁷ CFU, a viable cell count of between about 1×10⁷ CFU and about 1×10¹⁰ CFU, a viable cell count of between about 1×10⁷ CFU and about 1×10⁹ CFU, a viable cell count of between about 1×10⁷ CFU and about 1×10⁸ CFU, a viable cell count of between about 1×10⁸ CFU and about 1×10¹⁰ CFU, a viable cell count of between about 1×10⁸ CFU and about 1×10⁹ CFU, or a viable cell count of between about 1×10⁹ CFU and about 1×10¹⁰ CFU) per composition or per cm². Other methods for determining the viable cell count of a microbial cell population grown as a biofilm may be utilized (e.g., quantification of cells using manual cell counting (e.g., using plating and colony forming unit (CFU) enumeration) or automated cell counting (e.g., flow-based cell counting), see e.g., Wilson et al., Res. Rev. J. Eng. Technol. 6, 2017).

Biofilm Phenotype

In any of the compositions and methods described herein, the microbes are in a biofilm state and exhibit a biofilm phenotype. A biofilm phenotype may be characterized by an aggregate of microbial cells enmeshed in a self-produced extracellular polymeric matrix, and usually attached to a substrate. In some embodiments of the dehydrated biofilm compositions provided herein, the microbes are in a biofilm state and exhibit a biofilm phenotype wherein the biofilm is attached to a substrate. In other embodiments, the microbes are in a biofilm state and exhibit a biofilm phenotype wherein the biofilm is attached to an interface. In other embodiments, the microbes are in a biofilm state and exhibit a biofilm phenotype wherein the microbes are attached to each other within a biofilm and are not attached to a substrate or interface. Microbes in a biofilm exhibit a multicellular lifestyle and group behavior. A microbe and the phenotype of a microbe in a biofilm state is distinct and unique from a microbe in a planktonic state. Differences can include gene expression, metabolism, growth rates, antimicrobial tolerance, or other behaviors. A biofilm phenotype (e.g., the biofilm phenotype in any of the dehydrated biofilm compositions described herein) can be microscopic (e.g., a microbial biofilm that is not visible to the naked eye) or macroscopic (e.g., a microbial biofilm that is visible to the naked eye). A biofilm (e.g., the biofilm in any of the dehydrated biofilm compositions described herein) can be attached to a surface or substrate. The surface or substrate can be living (e.g., epithelial cells) or nonliving (e.g., a stainless steel disc). In any of the dehydrated biofilm compositions provided herein, a microbial cell population, or group of microbes, is defined as exhibiting a biofilm phenotype when the microbial cell population is enmeshed in an extracellular matrix. Non-limiting examples of biofilm phenotypes and properties of biofilms are described herein. Additional properties of biofilms and biofilm phenotypes are contemplated.

The extracellular polymeric matrix is a defining feature of the biofilm phenotype and comprises high molecular weight polymers secreted by microorganisms. The extracellular polymeric matrix is self-produced by the microorganisms contained within a biofilm. The extracellular polymeric matrix of a biofilm can be comprised of polysaccharides and water. In some examples, the extracellular polymeric matrix further includes proteins (e.g., enzymes), DNA, lipids, carbohydrates, or heavy metals. The composition of the extracellular matrix can affect the porosity, density, water content, charge, sorption properties, hydrophobicity, and mechanical stability of a biofilm. Any of the components within the extracellular polymeric matrix can interact with any of the other components or with the microbes present in the biofilm, making the extracellular polymeric matrix dynamic and versatile. The composition of the extracellular matrix can differ significantly depending on the organism(s) and environmental conditions. The components of a biofilm's extracellular matrix can vary at any given point in time and changes in composition can be due to environmental factors, e.g., available nutrients, shear flow, or substrate material. The components of a biofilm extracellular polymeric matrix can be functional or nonfunctional. In some examples, a protein is functional and involved in a cell motility, secretion, ribosomal functionality, metabolism, cell wall or membrane biogenesis, defense response, or cell adhesion. Non-limiting examples of functional proteins include flagella, porins, lipoproteins, adhesins, antigens, or enzymes. Water is the main component of a biofilm extracellular polymeric matrix and, e.g., can makeup up to 97% of the extracellular polymeric matrix. The extracellular polymeric matrix of a biofilm can comprise between about 0% and about 5% polysaccharide (e.g., between about 1% and 3% polysaccharide), between about 0% and about 5% protein, between about 0% and about 5% DNA, between about 0% and about 5% lipids, and between about 0% and about 5% carbohydrates weight/weight or weight/volume. Additional aspects and examples of biofilm extracellular polymeric matrix compositions are contemplated.

A microbe that switches to the biofilm mode of existence can undergo a phenotypic shift in behavior in which suites of genes are differentially regulated. Additionally, a biofilm mode of existence can facilitate genetic mutation of a microbe and the exchange of genetic material between microbes in a biofilm. Changes in a genome or alterations to the expression and regulation of a microbe's genome in a biofilm can alter mRNA production and synthesis of protein in the microbe causing changes in structural characteristics, energy production, nutrient acquisition, waste disposal, and cellular components. Gene expression, protein content, metabolism, and other characteristics can differ between microbes in a biofilm. Intracellular protein content can differ from extracellular protein content in a biofilm. A gene of a microbe in a biofilm can be upregulated or downregulated differently than the microbe in a planktonic state. For example, gene function of a microbe in a biofilm can change significantly within minutes of transitioning to a biofilm state from a planktonic state. A biofilm can contain a single organism or can contain more than one organism. A biofilm can contain subpopulations of cells within a biofilm, e.g., persister cells can exist within a biofilm. Microbes can form a biofilm in response to various different factors, including cellular recognition of specific or non-specific attachment sites on a substrate, nutritional cues, or environmental cues. Examples of phenotypic characteristics and traits of biofilm microbes that can differ from planktonic microbes include morphology (e.g., a self-organized three-dimensional biofilm structure characteristic of a microbial biofilm), development (e.g., coordinated multicellular behavior in a microbial biofilm), biochemical and physiological properties (e.g., extracellular polysaccharide production of a microbial biofilm), behavior and products of behavior (e.g., substrate attachment of a biofilm). Additional aspects of biofilm phenotypes and properties of biofilms may include those described in, e.g., Wood et al., Appl. Environ. Microbol. 79: 7116-7121, 2013; Jiao et al., Appl. Environ. Microbol. 77: 5230-5237, 2011; Flemming et al., J. Bacteriol. 189: 7945-7947, 2007; McCarthy et al., Front. Cell. Infect. Microbiol. 5:1-9, 2015; Conlon et al., J. Bacteriol. 196: 4268-4275, 2014; Monds and O'Toole, Trends Microbiol. 17:73-87, 2009; Hall-Stoodley et al., Nat. Rev. Microbol. 2:95-108, 2004; Percival et al., Wound Repair Regen. 20:647-657, 2012; Zapotoczna et al., PLoS Pathog. 12:e1005671, 2016; and Martin-Rodriguez et al., BMC Microbiol. 14:102, 2014.

Methods for identifying and visualizing a biofilm phenotype may be utilized such as, e.g., visualization by light microscopy and, optionally, use of dye staining (see, e.g., Walker and Keevil, Int. Biodeterior. Biodegradation 34:223-236, 1994), or scanning electron microscopy (SEM) (see, e.g., Hung et al., MBio. 4:e00645-13, 2013). Other methods and techniques for measuring or characterizing biofilm properties may be utilized, and include, e.g., methods to assess biofilm mass, viability, metabolism, matrix composition, adhesion extent, or adhesion strength. Biofilm biomass can be measured using, e.g., crystal violet dye staining (see, e.g., Christensen et al., J. Clin. Microbiol. 22:996-1006, 1985; and Fletcher, Can. J. Microbiol. 23:1-6, 1977), weight measurements (see, e.g., Trulear and Characklis, J. Water Pollut. Con. F. 15:1288-1301, 1982; and Jackson et al., J. Prosthet. Dent. 112:988-993, 2014), electrochemical impedance spectroscopy (ECIS) (see, e.g., Dominguez-Benetton et al., Chem. Soc. Rev. 41:7228-7246, 2012), and ultrasonic time-domain reflectometry (UTDR) (see, e.g., Sim et al., J. Membrane Sci. 428:24-37, 2013). Biofilm metabolic activity can be measured using, e.g., dye staining, e.g., XTT stain (see, e.g., Ramage, J. Med. Microbiol. 65:259-260, 2016; and Ramage et al., Antimicrob. Agents Chemother. 45: 2475-2479, 2001), TTC stain (see, e.g., Sabaeifard et al., J. Microbiol. Methods 105:134-140, 2014), or resazurin alamar blue stain (see, e.g., Peeters et al., J. Microbiol. Methods 72:157-165, 2008). Biofilm cellular biomass can be measured using, e.g., q-PCR (see, e.g., Klein et al., Mol. Oral Microbiol. 27:350-61, 2012). Biofilm cell viability can be measured using, e.g., CFU calculations (see, e.g., Jin et al., Arch. Oral Biol. 49:789-798, 2004), PMA-qPCR (see, e.g., Chen and Chang, J. Appl. Microbiol. 109:623-634, 2010), or flow cytometry (see, e.g., Cerca et al., Can. J. Microbiol. 57:850-856, 2011; and Oliveira et al., FEMS Microbiol. Lett. 362, 2015). Biofilm matrix composition can be measured using, e.g., confocal laser scanning microscopy (CLSM) (see, e.g., Lawrence et al., J. Bacteriol. 173:6558-6567, 1991; Neu and Lawrence, Adv. Biochem. Eng. Biotechnol. 146:1-51, 2014), fluorescence correlation spectroscopy (FCS) (see, e.g., Peulen and Wilkinson, Environ. Sci. Technol. 45:3367-3373, 2011), or matrix extraction (e.g., using physical methods or using chemical methods) followed by a suitable analytical methods to measure protein content, extracellular DNA content, and other matrix components (see, e.g., Adav and Lee, J. Hazard Mater. 154:1120-1126, 2008; and Kunacheva and Stuckey, Water Res. 61:1-18, 2014). Biofilm adhesion strength and extent can be measured using, e.g., atomic force microscopy (AFM) (see, e.g., Boyd et al., J. Bacteriol. 196:2775-2788, 2014; and Ovchinnikova et al., Langmuir 29:4823-4829, 2013). Additional methods and techniques for measuring biofilm adhesion extent, adhesion strength, biomass, viability, metabolism, matrix composition, or other properties of biofilms and biofilm phenotypes are contemplated.

Substrates

Any of the dehydrated biofilm compositions described herein can be grown on a substrate. Biofilms can grow on a wide variety of substrates including substrates comprising various shapes, surfaces, materials, densities, and textures. Non-limiting examples of substrates that any of the dehydrated biofilm compositions provided herein can be grown on are described below. The substrate may influence toxicity, hydrophilicity, hydrophobicity, diffusion of oxygen or medium components or other substances, specific gravity, biofilm architecture (e.g., biofilm form, size, or thickness), biofilm porosity, biofilm extracellular matrix composition, initial attachment of microbes, biofilm adhesion (e.g., extent and duration of biofilm adhesion), and other biofilm phenotype characteristics (e.g., gene expression). The substrate may also influence the number of viable cells of the biofilm, the viability of the biofilm before, during, and after the preservation process, and the long-term stability of the dehydrated biofilm compositions described herein.

In some examples, the substrate comprises wood, metal, metal alloy, plastic, rubber (e.g., a natural rubber or a synthetic rubber), carpet, textile, or glass. In some examples, the substrate comprises stainless steel (e.g., 304 grade, 306 grade, 316L grade, 316 grade, or 416 grade stainless steel), titanium, titanium alloy, polypropylene, silica, silicone, Teflon®, polycarbonate, acrylonitrile butadiene styrene, aluminum, anodized aluminum alloy, brass, nitrile rubber, silicone rubber, chlorosulfonated polyethylene synthetic rubber, cast iron, cobalt-chrome, granite, marble, quartz, Terrazzo tile, vinyl polymer, polyvinyl chloride, chlorinated polyvinyl chloride, epoxy, carbon steel, copper, ductile iron, ethylene propylene diene monomer rubber, borosilicate glass, ceramic (e.g., porcelain), hydroxyapatite, Lucitone™, nickel, nylon, polyether ether ketone, polystyrene, polytetrafluoroethylene, polyurethane, Viton®, polyethylene (e.g., polyethylene terephthalate glycol, polyethylene terephthalate, low density polyethylene, high density polyethylene, or ultra-high-molecular-weight polyethylene), methyl methacrylate, cement, sand, grout, paint, sealant, fiberglass, polyester, Lexan®, Plexiglas®, Kydex®, acrylic, or Aclar®. In other examples, the substrate comprises dextran, cellulose, or collagen. In other examples, the substrate comprises a polysaccharide or a disaccharide (e.g., glycosaminoglycan). In some embodiments, the substrate is a flexible material, e.g., a material with the ability to bend or compress without cracking. In some examples, the substrate is a rigid or non-flexible material, e.g., a material that is not bendable. Flexible and rigid, non-flexible materials may be utilized.

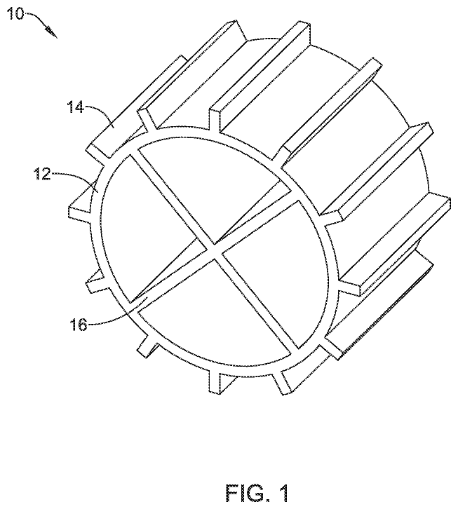

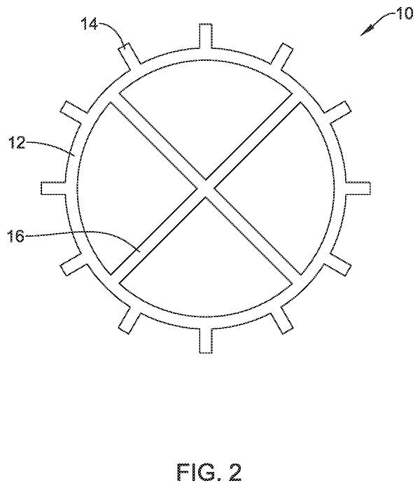

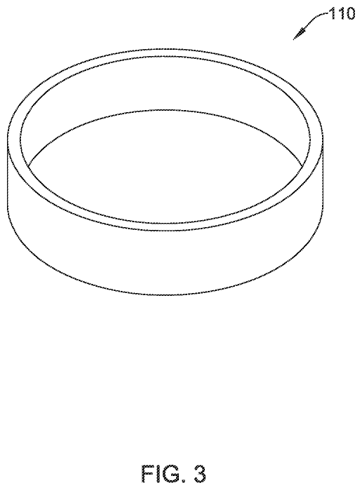

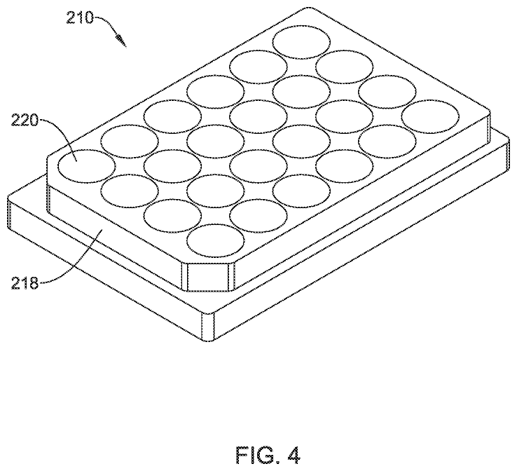

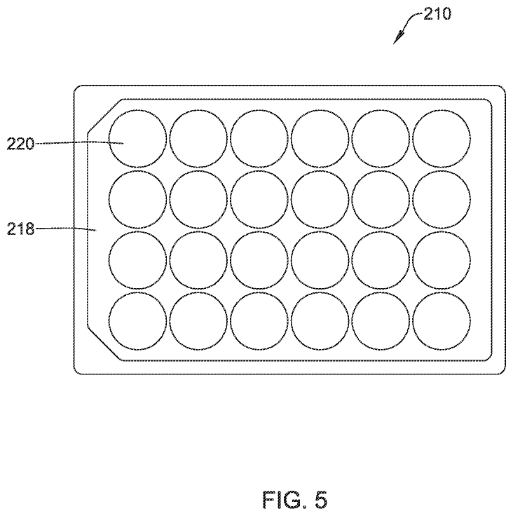

In some embodiments, the substrate is a surface of a container. In some examples, the substrate is the inside of a vial or the inside of a well of a microtiter plate. In other examples, the substrate is a sphere, e.g., a polypropylene, polyethylene, or polycarbonate sphere. In some examples, the substrate is a sphere, a rod, a cylinder, a test tube, a peg, a disc, or a rectangle. In some examples, the substrate is a Kaldnes-type carrier (e.g., a K1 carrier). A variety of Kaldnes-type carriers and their characteristics may be utilized. A non-limiting example of a Kaldnes-type carrier 10 is shown in FIG. 1 and FIG. 2. As illustrated in the FIGS. 1 and 2, a Kaldnes-type carrier 10 comprises a circular band 12 with a plurality of protrusions 14 along the exterior surface and one or more protrusions 16 extending across the diameter of the internal space of the circular band 12. In some examples, the substrate is a penicylinder (e.g., a flat face penicylinder or a beveled edge penicylinder). In other examples, the substrate is a microcarrier, e.g., a glass microcarrier or the like. In some examples, a substrate with a greater surface area may be desirable and the substrate is porous or perforated, e.g., a porous dextran microcarrier. In some examples, a porous or non-porous substrate may be desirable in order to replicate a real world application, e.g., various uses of disinfectants. Other substrates for biofilm growth are contemplated. For example, FIG. 3 illustrates a substrate 110 taking the form of a disc or disc-like tray. In at least some instances, the substrate 110 may resemble a contact lens container. As such, the substrate 110 may include a rounded/domed or tapered well. FIGS. 4-5 illustrate a substrate 210 taking the form of a plate 218 having a plurality of wells 220 formed therein. As can be appreciated by those skilled in the art, a specific substrate may be desirable for an organism or for an application.

Dehydrated biofilm assemblies are contemplated that include a substrate (e.g., like those disclosed herein) with a dehydrated biofilm disposed thereon or otherwise secured thereto. This may include securing the dehydrated biofilm to one or more surfaces of the substrate. In general, the dehydrated biofilm includes a population of micro-organisms (e.g., such as those disclosed herein and which may be disposed in a matrix as disclosed herein). The dehydrated biofilm is configured to retain securement to the substrate and exhibit a biofilm phenotype upon hydration.

In any of the dehydrated biofilm compositions described herein, the substrate can have various dimensions depending on its shape and features. Non-limiting examples of substrate material, shape, dimensions, and other features are described below.

In some examples, the substrate is a stainless steel (e.g., Type 304 stainless steel comprising at least 18% chromium and 8% nickel) penicylinder. In other examples, the substrate is a porcelain penicylinder. In some examples, the penicylinder has an outer diameter between about 5 mm and about 15 mm (e.g., between about 5 mm and about 13 mm, between about 5 mm and about 11 mm, between about 5 mm and about 9 mm, between about 5 mm and about 7 mm, between about 5 mm and about 6 mm, between about 6 mm and about 12 mm, between about 6 mm and about 10 mm, between about 6 mm and about 8 mm, between about 7 mm and about 15 mm, between about 7 mm and about 13 mm, between about 7 mm and about 11 mm, between about 7 mm and about 9 mm, between about 8 mm and about 12 mm, between about 8 mm and about 10 mm, between about 9 mm and about 15 mm, between about 9 mm and about 13 mm, between about 9 mm and about 11 mm, between about 10 mm and about 15 mm, between about 10 mm and about 12 mm, between about 12 mm and about 15 mm, or between about 13 mm and about 15 mm). In some examples, the penicylinder has an inner diameter between about 3 mm and about 13 mm (e.g., between about 3 mm and about 11 mm, between about 3 mm and about 9 mm, between about 3 mm and about 7 mm, between about 3 mm and about 5 mm, between about 4 mm and about 9 mm, between about 4 mm and about 7 mm, between about 4 mm and about 5 mm, between about 5 mm and about 9 mm, between about 5 mm and about 7 mm, between about 6 mm and about 9 mm, between about 6 mm and about 8 mm, between about 7 mm and about 9 mm, between about 8 mm and about 10 mm, between about 9 mm and about 11 mm, between about 10 mm and about 12 mm, or between about 11 mm and about 13 mm). In some examples, the penicylinder has a length between about 5 mm and about 15 mm (e.g., between about 5 mm and about 13 mm, between about 5 mm and about 11 mm, between about 5 mm and about 9 mm, between about 5 mm and about 7 mm, between about 5 mm and about 6 mm, between about 6 mm and about 12 mm, between about 6 mm and about 10 mm, between about 6 mm and about 8 mm, between about 7 mm and about 15 mm, between about 7 mm and about 13 mm, between about 7 mm and about 11 mm, between about 7 mm and about 9 mm, between about 8 mm and about 12 mm, between about 8 mm and about 10 mm, between about 9 mm and about 15 mm, between about 9 mm and about 13 mm, between about 9 mm and about 11 mm, between about 10 mm and about 15 mm, between about 10 mm and about 12 mm, between about 12 mm and about 15 mm, or between about 13 mm and about 15 mm). In some embodiments of the dehydrated biofilm compositions described herein, the substrate is a stainless steel penicylinder with an outer diameter of between about 7 mm and about 9 mm, an inner diameter of between about 5 mm and about 7 mm, and a length of between about 9 mm to about 11 mm. In other embodiments, the substrate is a porcelain penicylinder with an outer diameter of between about 7 mm and about 9 mm, an inner diameter of between about 5 mm and about 7 mm, and a length of between about 9 mm to about 11 mm.

In some examples, the substrate is a glass rectangle (e.g., a borosilicate glass cover slip or a borosilicate glass slide). In some examples, the substrate is a carpet rectangle, a stainless steel rectangle, a ceramic rectangle, or a wood rectangle. In some examples, the rectangle substrate can have a length and/or width of between about 2.5 mm and about 500 mm (e.g., between about 5 mm and about 400 mm, between about 5 mm and about 300 mm, between about 5 mm and about 200 mm, between about 5 mm and about 100 mm, between about 5 mm and about 50 mm, between about 5 mm and about 25 mm, between about 5 mm and about 15 mm, between about 10 mm and about 100 mm, between about 50 mm and about 300 mm, between about 50 mm and about 200 mm, between about 50 mm and about 150 mm, between about 100 mm and about 500 mm, between about 100 mm and about 250 mm, between about 100 mm and about 150 mm, between about 200 mm and about 500 mm, between about 200 mm and about 400 mm, between about 200 mm and about 300 mm, between about 300 mm and about 500 mm, between about 300 mm and about 400 mm, between about 400 mm and about 500 mm, between about 20 mm and about 100 mm, between about 20 mm and about 90 mm, between about 20 mm and about 80 mm, between about 20 mm and about 70 mm, between about between about 20 mm and about 60 mm, between about 20 mm and about 50 mm, between about 20 mm and about 40 mm, between about 20 mm and about 30 mm, between about 30 mm and about 100 mm, between about 30 mm and about 90 mm, between about 30 mm and about 80 mm, between about 30 mm and about 70 mm, between about 30 mm and about 60 mm, between about 30 mm and about 50 mm, between about 30 mm and about 40 mm, between about 40 mm and about 100 mm, between about 40 mm and about 90 mm, between about 40 mm and about 80 mm, between about 40 mm and about 70 mm, between about 40 mm and about 60 mm, between about 40 mm and about 50 mm, between about 50 mm and about 100 mm, between about 50 mm and about 90 mm, between about 50 mm and about 80 mm, between about 50 mm and about 70 mm, between about 50 mm and about 60 mm, between about 60 mm and about 100 mm, between about 60 mm and about 90 mm, between about 60 mm and about 80 mm, between about 60 mm and about 70 mm, between about 70 mm and about 100 mm, between about 70 mm and about 90 mm, between about 70 mm and about 80 mm, between about 80 mm and about 100 mm, between about 80 mm and about 90 mm, or between about 90 mm and about 100 mm). In some examples, the rectangle substrate can have a thickness of between about 0.05 mm and about 10 mm (e.g., between about 0.05 mm and about 0.15 mm, between about 0.05 mm and about 0.1 mm, between about 0.08 mm and about 0.13 mm, between about 0.1 mm and about 0.2 mm, between about 0.13 mm and about 0.17 mm, between about 0.16 mm and about 0.19 mm, between about 0.19 mm and about 0.25 mm, between about 0.25 mm and about 2 mm, between about 0.25 mm and about 1.75 mm, between about 0.25 mm and about 1.5 mm, between about 0.25 mm and about 1.25 mm, between about 0.25 mm and about 1 mm, between about 0.25 mm and about 0.75 mm, between about 0.25 mm and about 0.5 mm, between about 0.25 mm and about 0.35 mm, between about 0.35 mm and about 0.45 mm, between about 0.45 mm and about 0.55 mm, between about 0.5 mm and about 0.75 mm, between about 0.75 mm and about 1.5 mm, between about 0.75 mm and about 1.25 mm, between about 0.75 mm and about 1 mm, between about 0.8 mm and about 1.2 mm, between about 0.8 mm and about 1 mm, between about 0.9 mm and about 1.1 mm, between about 1 mm and about 1.5 mm, between about 1 mm and about 1.25 mm, between about 1.1 mm and about 1.3 mm, between about 1.5 mm and about 3 mm, between about 2 mm and about 3 mm, between about 3 mm and about 4 mm, between about 4 mm and about 5 mm, between about 5 mm and about 6 mm, between about 6 mm and about 7 mm, between about 7 mm and about 8 mm, between about 8 mm and about 9 mm, or between about 9 mm and about 10 mm). In some embodiments of the dehydrated biofilm compositions described herein, the substrate is a glass rectangle with a width of between about 23 mm and about 27 mm, a length of between about 73 mm and about 77 mm, and a thickness of between about 1.0 mm to about 1.2 mm. In other embodiments, the substrate is a glass rectangle with a width of between about 23 mm and about 27 mm, a length of between about 23 mm and about 27 mm, and a thickness of between about 0.4 mm to about 0.7 mm. In some embodiments of the dehydrated biofilm compositions described herein, the substrate is a glass rectangle with a width of between about 45 mm and about 55 mm, a length of between about 45 mm and about 55 mm, and a thickness of between about 1.0 mm to about 1.2 mm. In other embodiments, the substrate is a glass rectangle with a width of between about 45 mm and about 55 mm, a length of between about 45 mm and about 55 mm, and a thickness of between about 0.4 mm to about 0.7 mm.

In some examples, the substrate is a disc (e.g., a borosilicate glass disc, a stainless steel disc, or a disc comprising any of the exemplary materials provided herein). In some examples, the disc substrate can have a diameter of between about 5 mm and about 50 mm (e.g., between about 5 mm and about 45 mm, between about 5 mm and about 40 mm, between about 5 mm and about 35 mm, between about 5 mm and about 30 mm, between about 5 mm and about 25 mm, between about 5 mm and about 20 mm, between about 5 mm and about 15 mm, between about 5 mm and about 10 mm, between about 9 mm and about 15 mm, between about 9 mm and about 13 mm, between about 9 mm and about 11 mm, between about 10 mm and about 50 mm, between about 10 mm and about 40 mm, between about 10 mm and about 30 mm, between about 10 mm and about 20 mm, between about 10 mm and about 15 mm, between about 12 mm and about 15 mm, between about 12 mm and about 14 mm, between about 12 mm and about 13 mm, between about 20 mm and about 50 mm, between about 20 mm and about 40 mm, between about 20 mm and about 30 mm, between about 20 mm and about 25 mm, between about 30 mm and about 50 mm, between about 30 mm and about 40 mm, between about 30 mm and about 35 mm, between about 40 mm and about 50 mm, between about 40 mm and about 45 mm, or between about 45 mm and about 50 mm). In some examples, the disc substrate can have a thickness of between about 0.1 mm and about 10 mm (e.g., between about 0.1 mm and about 9 mm, between about 0.1 mm and about 8 mm, between about 0.1 mm and about 7 mm, between about 0.1 mm and about 6 mm, between about 0.1 mm and about 5 mm, between about 0.1 mm and about 4 mm, between about 0.1 mm and about 3 mm, between about 0.1 mm and about 2 mm, between about 0.1 mm and about 1 mm, between about 0.5 mm and about 1.5 mm, between about 0.5 mm and about 1.25 mm, between about 0.5 mm and about 1 mm, between about 0.5 mm and about 0.75 mm, between about 0.75 mm and about 1 mm, between about 0.75 mm and about 0.95 mm, between about 0.75 mm and about 0.9 mm, between about 0.75 mm and about 0.85 mm, between about 1 mm and about 2 mm, between about 2 mm and about 3 mm, between about 3 mm and about 4 mm, between about 3.5 mm and about 4 mm, between about 3.75 mm and about 3.85 mm, between about 4 mm and about 5 mm, between about 5 mm and about 6 mm, between about 6 mm and about 7 mm, between about 7 mm and about 8 mm, between about 8 mm and about 9 mm, or between about 9 mm and about 10 mm). In some embodiments of the dehydrated biofilm compositions described herein, the substrate is a borosilicate glass disc with a diameter of between about 12.5 mm and about 12.9 mm, and a thickness of between about 3.6 mm to about 4.0 mm. In other embodiments, the substrate is a stainless steel disc with a diameter of between about 9.8 mm and about 10.2 mm, and a thickness of between about 0.6 mm to about 1 mm.

Other examples of substrate material, shape, dimensions, and other features are contemplated. As can be appreciated by one skilled in the art, a certain substrate can be selected depending on the specific embodiment and application.

In some examples, the substrate is coated with a substance. The substance that coats the substrate can be a protein, a cell, an organism, a small molecule, a compound, or a chemical. In some examples, the substrate is coated with hydroxyapatite or titanium dioxide. In other examples, the substrate is coated with epithelial cells. In some examples, the substance coating the substrate can be negatively or positively charged. In some examples, the substance coating the substrate is intended to increase cell adhesion. In some examples, the substrate comprises an adhesive substance on at least one side.

The substrates can comprise an inactive material or an active material. An inactive material is one that is non-reactive when in contact or proximity to any of the biofilm compositions described herein and does not elicit a response from any of the biofilm compositions described herein. An active material is one that is dynamic or reactive when in contact or proximity to any of the biofilm compositions, or elicits a response from any of the biofilm compositions described herein. In some examples, the substrate can be negatively or positively charged.

In some examples, the substrate is a reservoir or reservoir compartment (e.g., the well of a multiple well plate (e.g., a 6 well plate, a 12 well plate, a 24 well plate, a 48 well plate, a 96 well plate, a 384 well plate, a 1536 well plate), a test tube (e.g., a culture tube, a centrifuge tube, a microcentrifuge tube, a PCR tube, a conical tube, or a freestanding tube), or a vial (e.g., a screw-thread vial, a snap-cap vial, or a crimp-top vial)). In some examples, the reservoir or reservoir compartments comprise one or more of the following materials: polyethylene (e.g., polyethylene terephthalate), polystyrene, polypropylene, polycarbonate, cyclo-olefin, quartz, glass, stainless steel, and titanium dioxide.

Any of the reservoir compartments described herein can be further coated with a substance. The substance coating a reservoir compartment can be an active or an inactive substance. An inactive substance is one that is non-reactive when in contact or proximity to any of the biofilm compositions described herein and does not elicit a response from any of the biofilm compositions described herein. An active substance is one that is dynamic or reactive when in contact or proximity to any of the biofilm compositions, or elicits a response from any of the biofilm compositions described herein. In some examples, a reservoir compartment is coated with a chemical, a compound, a plastic, a metal, a polymer, a protein, a nucleic acid, or a cell. In some examples, the coating is a hydroxyapatite or titanium dioxide. In other examples, the coating is concrete. In some examples, the coating is eukaryotic cells (e.g., human epithelial cells) or bacteria cells (e.g., bacteria found within the gastrointestinal tract of a human). In some embodiments, the reservoir compartment material or coating are intended to mimic conditions that biofilms encounter in real world conditions (e.g., conditions in a human organism, in a manufacturing facility, in a water treatment facility, or in an agricultural setting). In other embodiments, the reservoir compartment material or coating (e.g., an active material or coating) are intended to cause a response (e.g., increased attachment) from the biofilm composition. Other materials and coatings are contemplated. Other reasons for including a material or coating are contemplated. As can be appreciated by one skilled in the art, a certain material or coating can be selected depending on the specific embodiment and application.

Stabilizing Agents

When included in any of the dehydrated biofilm compositions provided herein, a stabilizing agent can act as a lyoprotectant, a cryoprotectant, a buffering agent, an antioxidant, or a bulking agent. In some examples, a stabilizing agent improves the stability of the composition through more than one (e.g., two, three, or four) mechanism, e.g., by acting as a lyoprotectant and a cryoprotectant, or by acting as a lyoprotectant and an antioxidant. Non-limiting examples of stabilizing agents that can be included in any of the dehydrated biofilm compositions provided herein include: a sugar (e.g., a monosaccharide, a disaccharide, a reducing sugar, or a non-reducing sugar), a polyol, a polymer (e.g., an oligosaccharide, a polysaccharide, a cellulose-derivative, or a synthetic polymer), an antioxidant, an amino acid, a surfactant, and a buffer. Surfactants can be present in any of the compositions of the disclosure, e.g., to stabilize and/or enhance the solubility of other constituents. Buffers can be included in any of the compositions of the disclosure, e.g., to stabilize other constituents and/or control pH.

Non-limiting examples of sugars that can be included in any of the compositions provided herein include: glucose, fructose, xylose, arabinose, sorbose, mannose, rhamnose, galactose, trehalose, maltose, lactose, sucrose, melibiose, maltulose, iso-maltulose, and lactulose.

Non-limiting examples of polymers that can be included in any of the compositions provided herein include: raffinose, stachyose, melezitose, mannotriose, maltodextrin, dextran, starch, inulin, ficoll, alginate, chitosan, methylcellulose, hydroxypropyl cellulose, hydroxyethyl cellulose, hypromellose, xanthan gum, guar gum, pectin, carrageen, galactomannan, gellan gum, cellulose acetate phthalate, carboxy-methyl-cellulose, a salt of alginic acid (e.g., sodium alginate), hydroxyl propyl methyl cellulose, gum acacia, locust bean gum, hydroxyethyl cellulose, hydroxypropyl methylcellulose, polyvinyl alcohol, polyvinyl pyrrolidone, gelatin (e.g., hydrolyzed gelatin and unhydrolyzed gelatin), and polyglycolic acid.

Non-limiting examples of polyols that can be included in any of the compositions provided herein include: sorbitol, arabitol, xylitol, mannitol, erythritol, threitol, and glycerol.

Non-limiting examples of antioxidants that can be included in any of the compositions provided herein include: ascorbic acid, citric acid, acetic acid, a tocopherol, propyl gallate, tertiary butylhydroquinone, butylated hydroxyanisole, and butylated hydroxytoluene.

Non-limiting examples of amino acids that can be included in any of the compositions provided herein include: glycine betaine, sodium glutamate, cysteine, cystine, histidine, and methionine.

Non-limiting examples of buffers that can be included in any of the compositions provided herein include: a potassium phosphate (e.g., monopotassium phosphate), a sodium phosphate (e.g., monosodium phosphate and disodium phosphate), sodium acetate, sodium citrate, sodium succinate, histidine, imidazole, ammonium bicarbonate, a carbonate, [Tris(hydroxymethyl)methylamino]propanesulfonic acid (TAPS), 2-(Bis(2-hydroxyethyl)amino)acetic acid (Bicine), Tris(hydroxymethyl)aminomethane (Tris), 3-[N-Tris(hydroxymethyl)methylamino]-2-hydroxypropanesulfonic acid (Tricine), 3-[N-Tris(hydroxymethyl)methylamino]-2-hydroxypropanesulfonic acid (TAPSO), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), Piperazine-N,N′-bis(2-ethanesulfonic acid) (PIPES), Dimethylarsenic acid (Cacodylate), 2-(N-morpholino)ethanesulfonic acid (MES), and N-cyclohexyl-2-aminoethanesulfonic acid (CHES).

Non-limiting examples of surfactants that can be included in any of the compositions provided herein include: a polysorbate (e.g., polysorbate 20, polysorbate 40, polysorbate 60, or polysorbate 80), a poloxamer (e.g., PLURONICS™), polyethylene glycol, polypropylene glycol, polyethylene glycol/polypropylene glycol block copolymers, polyethylene glycol alkyl ethers, polypropylene glycol alkyl ethers, and polyethylene glycol/polypropylene glycol ether block copolymers.

Non-limiting examples of other stabilizing agents that can be included in any of the compositions provided herein include: milk (e.g., skimmed milk), liquid growth medium, and propylene glycol. Other stabilizing agents that can be included in any of the dehydrated biofilm compositions are contemplated.

Any of the dehydrated biofilm compositions described herein can comprise between about 0.01 mg to about 1,000 mg (e.g., between about 0.01 mg to about 100 mg, between about 0.01 mg to about 12 mg, between about 0.01 mg to about 6 mg, between about 0.01 mg to about 1 mg, between about 0.05 mg to about 1 mg, between about 0.05 mg to about 4.5 mg, between about 0.05 mg to about 11 mg, between about 0.5 mg to about 130 mg, between about 0.5 mg to about 640 mg, between about 0.5 mg to about 200 mg, between about 0.25 mg to about 100 mg, between about 0.25 mg to about 80 mg, between about 0.25 mg to about 60 mg, between about 0.25 mg to about 40 mg, between about 0.25 mg to about 20 mg, between about 0.25 mg to about 10 mg, between about 0.25 mg to about 5 mg, between about 0.25 mg to about 2.5 mg, between about 0.25 mg to about 2 mg, between about 0.25 mg to about 1 mg, between about 0.5 mg to about 10 mg, between about 0.5 mg to about 9 mg, between about 0.5 mg to about 8 mg, between about 0.5 mg to about 6 mg, between about 0.5 mg to about 5 mg, between about 0.5 mg to about 4 mg, between about 0.5 mg to about 3 mg, between about 0.5 mg to about 2 mg, between about 0.5 mg to about 1 mg, between about 0.5 mg to about 0.75 mg, between about 0.75 mg to about 1 mg, between about 1 mg to about 2 mg, between about 1.25 mg to about 1.75 mg, between about 0.25 mg to about 12 mg, between about 0.25 mg to about 8 mg, between about 0.25 mg to about 1.5 mg, between about 0.5 mg to about 60 mg, between about 0.5 mg to about 4.5 mg, between about 0.5 mg to about 11 mg, between about 0.5 mg to about 130 mg, between about 0.5 mg to about 640 mg, between about 0.5 mg to about 200 mg, between about 0.5 mg to about 2 mg, between about 0.5 mg to about 35 mg, between about 10 mg to about 30 mg, between about 10 mg to about 25 mg, between about 10 mg to about 20 mg, between about 10 mg to about 15 mg, between about 10 mg to about 12.5 mg, between about 10 mg to about 80 mg, between about 10 mg to about 70 mg, between about 10 mg to about 60 mg, between about 10 mg to about 50 mg, between about 10 mg to about 40 mg, between about 15 mg to about 25 mg, between about 17.5 mg to about 22.5 mg, between about 20 mg to about 30 mg, between about 20 mg to about 25 mg, between about 25 mg to about 45 mg, between about 25 mg to about 35 mg, between about 25 mg to about 30 mg, between about 30 mg to about 45 mg, between about 30 mg to about 40 mg, between about 30 mg to about 35 mg, between about 35 mg to about 40 mg, between about 45 mg to about 55 mg, between about 50 mg to about 100 mg, between about 50 mg to about 90 mg, between about 50 mg to about 80 mg, between about 50 mg to about 70 mg, between about 50 mg to about 60 mg, between about 50 mg to about 55 mg, between about 150 mg to about 300 mg, between about 150 mg to about 450 mg, between about 150 mg to about 250 mg, between about 150 mg to about 180 mg, between about 225 mg to about 500 mg, between about 225 mg to about 400 mg, between about 225 mg to about 300 mg, between about 600 mg to about 1,000 mg, between about 600 mg to about 900 mg, between about 20 mg to about 25 mg, between about 20 mg to about 40 mg, between about 20 mg to about 28 mg, between about 20 mg to about 37 mg, between about 750 mg to about 775 mg, between about 750 mg to about 825 mg, between about 800 mg to about 915 mg, between about 75 mg to about 105 mg, between about 75 mg to about 99 mg, between about 30 mg to about 60 mg, between about 30 mg to about 50 mg, between about 30 mg to about 40 mg, between about 4 mg to about 16 mg, between about 4 mg to about 12 mg, between about 4 mg to about 8 mg, between about 3 mg to about 9 mg, between about 3 mg to about 6 mg, between about 2 mg to about 6 mg, between about 2 mg to about 5 mg, between about 12 mg to about 15 mg, between about 12 mg to about 14 mg, between about 15 mg to about 20 mg, or between about 15 mg to about 18 mg) of a stabilizing agent (e.g., any of the exemplary stabilizing agents provided herein) per composition or per cm².

Residual Moisture Content & Stability

Any of the dehydrated biofilm compositions provided herein can comprise residual moisture content or residual water content (e.g., a measurable amount of water). Residual water content is the amount of water that remains in the composition after dehydration Similar to residual water content, residual moisture content includes not only the amount of remaining water but also other volatile substances that remain in the composition after dehydration. The residual water and moisture content have a significant impact on the stability of any of the dehydrated biofilm compositions described herein. The presence of residual water content can provide conditions for some metabolic activity to continue within the biofilm, contributing to degradation following dehydration and during storage. It may be desirable to have minimal residual water and moisture content.

In some examples of the compositions described herein the dehydrated biofilm composition can comprise between about 0% to about 25% (e.g., between about 0.001% to about 20%, between about 0.5% to about 25%, between about 0.5% to about 20%, between about 0.5% to about 15%, between about 0.5% to about 10%, between about 0.5% to about 5%, between about 1% to about 25%, between about 1% to about 20%, between about 1% to about 15%, between about 1% to about 10%, between about 1% to about 5%, between about 1% to about 4%, between about 2% to about 25%, between about 2% to about 20%, between about 2% to about 15%, between about 2% to about 10%, between about 2% to about 5%, between about 2% to about 4%, between about 3% to about 25%, between about 3% to about 20%, between about 3% to about 15%, between about 3% to about 10%, between about 3% to about 5%, between about 4% to about 25%, between about 4% to about 20%, between about 4% to about 15%, between about 4% to about 10%, between about 4% to about 8%, between about 4% to about 5%, between about 1.5% to about 25%, between about 1.5% to about 20%, between about 1.5% to about 15%, between about 1.5% to about 10%, between about 1.5% to about 5%, between about 2.5% to about 25%, between about 2.5% to about 20%, between about 2.5% to about 15%, between about 2.5% to about 10%, or between about 2.5% to about 5% water) of residual water content or residual moisture content by weight of the composition (e.g., w/w). In at least some instances, the residual water content or residual moisture content of any of the dehydrated biofilm compositions provided herein is less than about 15% (e.g., less than about 12%, less than about 10%, less than about 9%, less than about 8%, less than about 7%, less than about 6%, less than about 5%, less than about 4.5%, less than about 4%, less than about 3.5%, less than about 3%, less than about 2.5%, less than about 2%, less than about 1.5%, less than about 1%, or less than about 0.5%) by weight of the composition (e.g., w/w). In some instances, the residual water content or residual moisture content is less than about 5% by weight of the composition (e.g., w/w).

Any of the dehydrated biofilm compositions described herein can comprise between about 0 mg to about 500 mg (e.g., between about 0.25 mg to about 500 mg, between about 0.25 mg to about 400 mg, between about 0.25 mg to about 300 mg, between about 0.25 mg to about 200 mg, between about 0.25 mg to about 100 mg, between about 0.25 mg to about 50 mg, between about 0.25 mg to about 25 mg, between about 5 mg to about 500 mg, between about 5 mg to about 400 mg, between about 5 mg to about 300 mg, between about 5 mg to about 200 mg, between about 5 mg to about 100 mg, between about 5 mg to about 50 mg, between about 5 mg to about 25 mg, between about 15 mg to about 500 mg, between about 15 mg to about 400 mg, between about 15 mg to about 300 mg, between about 15 mg to about 200 mg, between about 15 mg to about 100 mg, between about 15 mg to about 50 mg, between about 15 mg to about 25 mg, between about 50 mg to about 500 mg, between about 50 mg to about 400 mg, between about 50 mg to about 300 mg, between about 50 mg to about 250 mg, between about 50 mg to about 200 mg, between about 50 mg to about 150 mg, between about 50 mg to about 100 mg, between about 100 mg to about 500 mg, between about 100 mg to about 450 mg, between about 100 mg to about 400 mg, between about 100 mg to about 350 mg, between about 100 mg to about 300 mg, between about 100 mg to about 250 mg, between about 100 mg to about 200 mg, between about 100 mg to about 150 mg, between about 150 mg to about 500 mg, between about 150 mg to about 450 mg, between about 150 mg to about 400 mg, between about 150 mg to about 350 mg, between about 150 mg to about 300 mg, between about 150 mg to about 250 mg, between about 150 mg to about 200 mg, between about 200 mg to about 500 mg, between about 200 mg to about 450 mg, between about 200 mg to about 400 mg, between about 200 mg to about 350 mg, between about 200 mg to about 300 mg, between about 200 mg to about 250 mg, between about 250 mg to about 500 mg, between about 250 mg to about 450 mg, between about 250 mg to about 400 mg, between about 250 mg to about 350 mg, between about 250 mg to about 300 mg, between about 300 mg to about 500 mg, between about 300 mg to about 450 mg, between about 300 mg to about 400 mg, between about 300 mg to about 350 mg, between about 350 mg to about 500 mg, between about 350 mg to about 450 mg, between about 350 mg to about 400 mg, between about 400 mg to about 500 mg, or between about 400 mg to about 450 mg) of residual water content per composition or per cm². Other methods for determining residual water content may include the Karl Fisher titration method (see, e.g., Krasucka et al., Acta Pol. Pharm. 69: 1364-1367, 2012 and Reh et al., Food Chem. 86: 457-464, 2004). Other methods for determining residual moisture content may include loss-on-drying or gravimetric methodology (see, e.g., May et al., Cryobiology 26: 277-284, 1989), and using near-infrared spectroscopy (see, e.g., Zheng et al., J. Pharm. Biomed. Anal. 46: 592-596, 2008).

Any of the dehydrated biofilm compositions provided herein can also be characterized by water activity. In some examples of the compositions described herein the dehydrated biofilm composition can exhibit a water activity of less than about 0.9 Aw (e.g., less than about 0.85 Aw, less than about 0.8 Aw, less than about 0.75 Aw, less than about 0.7 Aw, less than about 0.65 Aw, less than about 0.6 Aw, less than about 0.55 Aw, less than about 0.5 Aw, less than about 0.45 Aw, less than about 0.4 Aw, less than about 0.35 Aw, less than about 0.3 Aw, less than about 0.25 Aw, less than about 0.2 Aw, less than about 0.15 Aw, less than about 0.1 Aw, or less than about 0.05 Aw). Methods for determining the water activity of a composition at a given temperature may include, e.g., using a resistive electrolytic hygrometer, a capacitance hygrometer, or a dew point hygrometer.

The loss of viability of any of the dehydrated biofilm compositions described herein can be calculated by subtracting the number of log CFU (e.g., the number of log CFU per composition or per cm²) of a dehydrated biofilm composition (e.g., any of the dehydrated biofilm compositions provided herein) at a point in time (e.g., at about 30 days, at about 60 days, at about 90 days, at about 180 days, at about 365 days, or at about 730 days) from the number of log CFU of the composition immediately following dehydration. The resulting number is the loss of viability of a dehydrated biofilm composition over a period of time (e.g., over a period of about 30 days, over a period of about 60 days, over a period of about 90 days, over a period of about 180 days, over a period of about 365 days, or over a period of about 730 days).

The loss of viability of any of the dehydrated biofilm compositions described herein can be less than about 5 log CFU (e.g., less than about 4.9 log CFU, less than about 4.8 log CFU, less than about 4.7 log CFU, less than about 4.6 log CFU, less than about 4.5 log CFU, less than about 4.4 log CFU, less than about 4.3 log CFU, less than about 4.2 log CFU, less than about 4.1 log CFU, less than about 4 log CFU, less than about 3.9 log CFU, less than about 3.8 log CFU, less than about 3.7 log CFU, less than about 3.6 log CFU, less than about 3.5 log CFU, less than about 3.4 log CFU, less than about 3.3 log CFU, less than about 3.2 log CFU, less than about 3.1 log CFU, less than about 3 log CFU, less than about 2.9 log CFU, less than about 2.8 log CFU, less than about 2.7 log CFU, less than about 2.6 log CFU, less than about 2.5 log CFU, less than about 2.4 log CFU, less than about 2.3 log CFU, less than about 2.2 log CFU, less than about 2.1 log CFU, less than about 2 log CFU, less than about 1.9 log CFU, less than about 1.8 log CFU, less than about 1.7 log CFU, less than about 1.6 log CFU, less than about 1.5 log CFU, less than about 1.4 log CFU, less than about 1.3 log CFU, less than about 1.2 log CFU, less than about 1.1 log CFU, less than about 1 log CFU, less than about 0.9 log CFU, less than about 0.8 log CFU, less than about 0.7 log CFU, less than about 0.6 log CFU, less than about 0.5 log CFU, less than about 0.4 log CFU, less than about 0.3 log CFU, less than about 0.2 log CFU, or less than about 0.1 log CFU) per composition or per cm² over a period of at least about 30 days (e.g., at least about 35 days, at least about 60 days, at least about 90 days, at least about 120 days, at least about 150 days, at least about 180 days, at least about 210 days, at least about 240 days, at least about 270 days, at least about 300 days, at least about 365 days, or at least about 730 days) at a temperature between about 2° C. to about 7° C. (e.g., about 2° C., about 3° C., about 4° C., about 5° C., about 6° C., or about 7° C.). In some embodiments, the loss of viability of the dehydrated biofilm composition is less than about 3 log CFU per composition or per cm² over a period of about 180 days at a temperature of about 5° C. In some embodiments, the loss of viability of the dehydrated biofilm composition is less than about 1.5 log CFU per composition or per cm² over a period of about 180 days at a temperature of about 5° C.

The loss of viability of any of the dehydrated biofilm compositions described herein can be less than about 5 log CFU (e.g., less than about 4.9 log CFU, less than about 4.8 log CFU, less than about 4.7 log CFU, less than about 4.6 log CFU, less than about 4.5 log CFU, less than about 4.4 log CFU, less than about 4.3 log CFU, less than about 4.2 log CFU, less than about 4.1 log CFU, less than about 4 log CFU, less than about 3.9 log CFU, less than about 3.8 log CFU, less than about 3.7 log CFU, less than about 3.6 log CFU, less than about 3.5 log CFU, less than about 3.4 log CFU, less than about 3.3 log CFU, less than about 3.2 log CFU, less than about 3.1 log CFU, less than about 3 log CFU, less than about 2.9 log CFU, less than about 2.8 log CFU, less than about 2.7 log CFU, less than about 2.6 log CFU, less than about 2.5 log CFU, less than about 2.4 log CFU, less than about 2.3 log CFU, less than about 2.2 log CFU, less than about 2.1 log CFU, less than about 2 log CFU, less than about 1.9 log CFU, less than about 1.8 log CFU, less than about 1.7 log CFU, less than about 1.6 log CFU, less than about 1.5 log CFU, less than about 1.4 log CFU, less than about 1.3 log CFU, less than about 1.2 log CFU, less than about 1.1 log CFU, less than about 1 log CFU, less than about 0.9 log CFU, less than about 0.8 log CFU, less than about 0.7 log CFU, less than about 0.6 log CFU, less than about 0.5 log CFU, less than about 0.4 log CFU, less than about 0.3 log CFU, less than about 0.2 log CFU, or less than about 0.1 log CFU) per composition or per cm² over a period of at least about 30 days (e.g., at least about 35 days, at least about 60 days, at least about 90 days, at least about 120 days, at least about 150 days, at least about 180 days, at least about 210 days, at least about 240 days, at least about 270 days, at least about 300 days, at least about 365 days, or at least about 730 days) at a temperature between about 20° C. to about 25° C. (e.g., about 20° C., about 21° C., about 22° C., about 23° C., about 24° C., or about 25° C.).

Methods of Preparing Dehydrated Biofilm Compositions

Also provided herein are methods of preparing a dehydrated biofilm composition that include (1) growing a biofilm composition on a substrate (e.g., any of the exemplary substrates described herein), (2) adding one or more stabilizing agent(s) to the biofilm composition, (3) freezing the biofilm composition, (4) dehydrating the biofilm composition by sublimation, (5) optionally dehydrating the biofilm composition by desorption, and (6) enclosing the dehydrated biofilm composition in a primary container (e.g., any of the primary containers described herein). In some embodiments, dehydrating the biofilm composition by desorption is required to prepare a dehydrated biofilm composition with satisfactory residual moisture content and stability properties. The methods provided herein are capable of preparing a dehydrated biofilm composition (e.g., any of the dehydrated biofilm compositions described herein) that exhibits a biofilm phenotype (e.g., a substrate attached microbial community enmeshed in a self-produced extracellular matrix) and has a viable cell count (e.g., between about 1×10⁴ CFU to about 1×10⁸ CFU, between about 1×10⁷ CFU to about 5×10⁸ CFU, or between about 1×10⁸ CFU to about 5×10⁹ CFU per composition or per cm²) upon rehydration. The methods described herein can prepare a dehydrated biofilm composition that is stably dehydrated and can be subsequently rehydrated without destroying the biofilm phenotype and irreversibly breaking down the three dimensional architecture, structure, and function of a biofilm. In particular, the methods described herein can prepare a dehydrated biofilm composition that remains attached to a substrate after rehydration.

Any of the dehydrated biofilm compositions provided herein can be dehydrated by lyophilization. Lyophilization is a process where water and other solvents are removed via sublimation. In any of the methods of dehydrating biofilm compositions provided herein, the microbial cell population and extracellular matrix is dehydrated thereby stopping or slowing the metabolic activity of the microbes and stably preserving the structure, function, and viability of the biofilm. Dehydration of a biofilm composition by lyophilization includes (1) freezing, (2) primary drying (e.g., dehydration by sublimation), and optionally, (3) secondary drying (e.g., dehydration by desorption). During the freezing step, solvent present in the biofilm composition is made solid by reducing the temperature below the solvent freezing point. After freezing, the biofilm composition is subjected to a primary drying phase where the majority of water content is removed by sublimation. After primary drying, the biofilm composition can optionally be subjected to a secondary drying phase. During the secondary drying phase, the biofilm composition can be subjected to an increased temperature in order to remove residual water content by desorption. The secondary drying phase is particularly important for the long-term stability of the dehydrated biofilm compositions. Reducing the residual moisture content of the dehydrated biofilm composition to a minimum may be desirable, however it must be balanced with not over drying the biofilm composition to a point where any degradation occurs or damage is inflicted on the biofilm compositions components (e.g., resulting in a loss of viability).

A biofilm composition can be grown on a substrate (e.g., any of the exemplary substrates described herein) according to a suitable method, see, e.g., Azeredo et al., Critical Reviews in Microbiology 43:313-351.

In some embodiments, a stabilizing agent (e.g., a lyoprotectant) is added to the biofilm composition prior to dehydration. In at least some instances, the biofilm composition is submerged in a solution containing stabilizing agent(s) before the dehydration process begins. In some examples, the biofilm composition is submerged in a solution containing stabilizing agent(s) for at least thirty seconds, at least one minute, at least 2 minutes, or at least 3 minutes before the dehydration process begins. In other embodiments, the biofilm composition can be submerged in the solution containing stabilizing agent(s) for between about 2 minutes and about 60 minutes (e.g., between about 2 minutes and about 55 minutes, between about 2 minutes and about 50 minutes, between about 2 minutes and about 45 minutes, between about 2 minutes and about 40 minutes, between about 2 minutes and about 35 minutes, between about 2 minutes and about 30 minutes, between about 2 minutes and about 25 minutes, between about 2 minutes and about 20 minutes, between about 2 minutes and about 15 minutes, between about 2 minutes and about 10 minutes, between about 2 minutes and about 5 minutes, between about 5 minutes and about 60 minutes, between about 5 minutes and about 55 minutes, between about 5 minutes and about 50 minutes, between about 5 minutes and about 45 minutes, between about 5 minutes and about 40 minutes, between about 5 minutes and about 35 minutes, between about 5 minutes and about 30 minutes, between about 5 minutes and about 25 minutes, between about 5 minutes and about 20 minutes, between about 5 minutes and about 15 minutes, between about 5 minutes and about 10 minutes, between about 5 minutes and about 8 minutes, between about 10 minutes and about 60 minutes, between about 10 minutes and about 55 minutes, between about 10 minutes and about 50 minutes, between about 10 minutes and about 45 minutes, between about 10 minutes and about 40 minutes, between about 10 minutes and about 35 minutes, between about 10 minutes and about 30 minutes, between about 10 minutes and about 25 minutes, between about 10 minutes and about 20 minutes, between about 10 minutes and about 15 minutes, between about 15 minutes and about 60 minutes, between about 15 minutes and about 55 minutes, between about 15 minutes and about 50 minutes, between about 15 minutes and about 45 minutes, between about 15 minutes and about 40 minutes, between about 15 minutes and about 35 minutes, between about 15 minutes and about 30 minutes, between about 15 minutes and about 25 minutes, between about 15 minutes and about 20 minutes, between about 20 minutes and about 60 minutes, between about 20 minutes and about 55 minutes, between about 20 minutes and about 50 minutes, between about 20 minutes and about 45 minutes, between about 20 minutes and about 40 minutes, between about 20 minutes and about 35 minutes, between about 20 minutes and about 30 minutes, between about 20 minutes and about 25 minutes, between about 30 minutes and about 60 minutes, between about 30 minutes and about 55 minutes, between about 30 minutes and about 50 minutes, between about 30 minutes and about 45 minutes, between about 30 minutes and about 40 minutes, or between about 30 minutes and about 35 minutes) before the dehydration process begins. In some examples, the biofilm composition is submerged in a solution containing stabilizing agent(s) for less than thirty seconds (e.g., less than 25 seconds) before the dehydration process begins. In other examples, the biofilm composition is submerged in a solution containing stabilizing agent(s) for more than 60 minutes before the dehydration process begins.

The solution containing stabilizing agent(s) can include between about 0.01% to about 60% (e.g., between about 0.01% and about 55%, between about 0.01% and about 50%, between about 0.01% and about 45%, between about 0.01% and about 40%, between about 0.01% and about 35%, between about 0.01% and about 30%, between about 0.01% and about 25%, between about 0.01% and about 20%, between about 0.01% and about 15%, between about 0.1% and about 10%, between about 0.1% and about 8%, between about 0.1% and about 6%, between about 0.1% and about 5%, between about 0.1% and about 4%, between about 0.1% and about 3%, between about 0.1% and about 2.0%, between about 0.1% and about 1.0%, between about 0.1% and about 0.5%, between about 0.5% and about 65%, between about 0.5% and about 60%, between about 0.5% and about 55%, between about 0.5% and about 50%, between about 0.5% and about 45%, between about 0.5% and about 40%, between about 0.5% and about 35%, between about 0.5% and about 30%, between about 0.5% and about 25%, between about 0.5% and about 20%, between about 0.5% and about 15%, between about 0.5% and about 10%, between about 0.5% and about 8%, between about 0.5% and about 6.0%, between about 0.5% and about 5.0%, between about 0.5% and about 4.0%, between about 0.5% and about 3.0%, between about 0.5% and about 2.0%, between about 0.5% and about 1.0%, between about 1% and about 60%, between about 1% and about 55%, between about 1% and about 50%, between about 1% and about 45%, between about 1% and about 40%, between about 1% and about 35%, between about 1% and about 30%, between about 1% and about 25%, between about 1% and about 20%, between about 1% and about 15%, between about 1% and about 10%, between about 1% and about 8%, between about 1% and about 6%, between about 1% and about 5%, between about 1% and about 4%, between about 1% and about 3%, between about 1% and about 2.5%, between about 1% and about 2%, between about 1% and about 1.5%, between about 2.5% and about 60%, between about 2.5% and about 55%, between about 2.5% and about 50%, between about 2.5% and about 45%, between about 2.5% and about 40%, between about 2.5% and about 35%, between about 2.5% and about 30%, between about 2.5% and about 25%, between about 2.5% and about 20%, between about 2.5% and about 15%, between about 2.5% and about 10%, between about 2.5% and about 8%, between about 2.5% and about 6%, between about 2.5% and about 5%, between about 5% and about 60%, between about 5% and about 55%, between about 5% and about 50%, between about 5% and about 45%, between about 5% and about 40%, between about 5% and about 35%, between about 5% and about 30%, between about 5% and about 25%, between about 5% and about 20%, between about 5% and about 15%, between about 5% and about 10%, between about 5% and about 8%, between about 10% and about 60%, between about 10% and about 55%, between about 10% and about 50%, between about 10% and about 45%, between about 10% and about 40%, between about 10% and about 35%, between about 10% and about 30%, between about 10% and about 25%, between about 10% and about 20%, between about 10% and about 15%, between about 10% and about 12.5%, between about 15% and about 60%, between about 15% and about 55%, between about 15% and about 50%, between about 15% and about 45%, between about 15% and about 40%, between about 15% and about 35%, between about 15% and about 30%, between about 15% and about 25%, between about 15% and about 20%, or between about 15% and about 17.5%) by weight (e.g., w/w or w/v) of any of the exemplary stabilizing agents provided herein. In some instances, the solution containing stabilizing agent(s) can include between about 0.1% to about 15% by weight (e.g., w/w or w/v) of any of the exemplary stabilizing agents provided herein.

In some embodiments, the solution containing stabilizing agent(s) can have a pH between about pH 4 to about pH 10 (e.g., between about pH 4 to about pH 9, between about pH 4 to about pH 8, between about pH 4 to about pH 7, between about pH 4 to about pH 6, between about pH 4 to about pH 5, between about pH 5 to about pH 10, between about pH 5 to about pH 9, between about pH 5 to about pH 8, between about pH 5 to about pH 7, between about pH 5 to about pH 6, between about pH 6 to about pH 10, between about pH 6 to about pH 9, between about pH 6 to about pH 8, between about pH 6 to about pH 7, between about pH 7 to about pH 10, between about pH 7 to about pH 9, between about pH 7 to about pH 8, between about pH 4.5 to about pH 9.5, between about pH 5.5 to about pH 8.5, between about pH 6.5 to about pH 7.5, between about pH 6 to about pH 6.5, between about pH 7 to about pH 7.5, between about pH 6.5 to about pH 7, or between about pH 7.5 to about pH 8).

In some embodiments, the biofilm composition is dehydrated while submerged in the stabilizing agent solution. In some embodiments, the excess stabilizing agent solution is removed before the biofilm composition is dehydrated. In some embodiments, the biofilm composition is submerged in a solution containing stabilizing agent(s) for between about 5 minutes to about 120 minutes (e.g., between about 60 minutes and about 120 minutes, between about 60 minutes and about 90 minutes, between about 60 minutes and about 75 minutes, between about 75 minutes and about 120 minutes, between about 75 minutes and about 105 minutes, between about 75 minutes and about 90 minutes, between about 90 minutes and about 120 minutes, between about 90 minutes and about 105 minutes, or between about 105 minutes and about 120 minutes) before dehydration, wherein the stabilizing agent(s) are absorbed and retained within the biofilm in an amount sufficient to improve the stability of the biofilm composition after dehydration, and then the excess solution is removed. Without being bound by theory, this may improve the dehydration process of the biofilm compositions described herein by reducing the volume of solvent (e.g., water) present thereby shortening the dehydration process and reducing the exposure of the biofilm composition to potentially harmful stressors such as temperature and vacuum pressure. In some embodiments, this can reduce the potential of damage to the three dimensional architecture, structure, and viability of the biofilm composition during the freezing and drying phases of the dehydration process.

In some embodiments, a biofilm composition and any of its components (e.g., water contained within any of the biofilm compositions described herein) can be frozen by placing the biofilm composition (e.g., any of the biofilm compositions described herein) in a lyophilizer with freezer capabilities. In some embodiments, a biofilm composition and any of its components (e.g., water contained within any of the biofilm compositions described herein) can be frozen by placing the biofilm composition (e.g., any of the biofilm compositions described herein) in a laboratory freezer (e.g., at a temperature between −20° C. and about −40° C.). In other embodiments, the biofilm composition can be frozen by placing it in an ultra low freezer (e.g., at a temperature between about −80° C. and about −90° C.). In other embodiments, the biofilm composition can be frozen by placing it in a cryogenic freezer (e.g., at a temperature between about −125° C. and about −150° C.). In other embodiments, the biofilm composition can be frozen by subjecting the biofilm composition or the vessel containing the biofilm composition to direct contact with liquid nitrogen (e.g., at a temperature between about −190° C. and about −200° C.). Additional methods and equipment for freezing may be utilized, e.g., flask shell freezing in a shell bath.

In some embodiments, a biofilm composition and any of its components (e.g., water contained within any of the biofilm compositions described herein) can be frozen by subjecting the biofilm composition to a temperature of between about 0° C. and about −200° C. (e.g., between about 0° C. and about −10° C., between about −10° C. and about −190° C., between about −10° C. and about −180° C., between about −10° C. and about −170° C., between about −10° C. and about −160° C., between about −10° C. and about −150° C., between about −10° C. and about −140° C., between about −10° C. and about −130° C., between about −10° C. and about −120° C., between about −10° C. and about −110° C., between about −10° C. and about −100° C., between about −10° C. and about −90° C., between about −10° C. and about −80° C., between about −10° C. and about −70° C., between about −10° C. and about −60° C., between about −10° C. and about −50° C., between about −10° C. and about −40° C., between about −10° C. and about −30° C., between about −10° C. and about −20° C., between about −10° C. and about −15° C., between about −20° C. and about −190° C., between about −20° C. and about −180° C., between about −20° C. and about −170° C., between about −20° C. and about −160° C., between about −20° C. and about −150° C., between about −20° C. and about −140° C., between about −20° C. and about −130° C., between about −20° C. and about −120° C., between about −20° C. and about −110° C., between about −20° C. and about −100° C., between about −20° C. and about −90° C., between about −20° C. and about −80° C., between about −20° C. and about −70° C., between about −20° C. and about −60° C., between about −20° C. and about −50° C., between about −20° C. and about −40° C., between about −20° C. and about −30° C., between about −20° C. and about −25° C., between about −30° C. and about −190° C., between about −30° C. and about −180° C., between about −30° C. and about −170° C., between about −30° C. and about −160° C., between about −30° C. and about −150° C., between about −30° C. and about −140° C., between about −30° C. and about −130° C., between about −30° C. and about −120° C., between about −30° C. and about −110° C., between about −30° C. and about −100° C., between about −30° C. and about −90° C., between about −30° C. and about −80° C., between about −30° C. and about −70° C., between about −30° C. and about −60° C., between about −30° C. and about −50° C., between about −30° C. and about −40° C., between about −60° C. and about −90° C., between about −70° C. and about −90° C., between about −70° C. and about −85° C., between about −70° C. and about −80° C., between about −70° C. and about −75° C., between about −75° C. and about −90° C., between about −75° C. and about −85° C., between about −75° C. and about −80° C., between about −80° C. and about −190° C., between about −80° C. and about −180° C., between about −80° C. and about −170° C., between about −80° C. and about −160° C., between about −80° C. and about −150° C., between about −80° C. and about −140° C., between about −80° C. and about −130° C., between about −80° C. and about −120° C., between about −80° C. and about −110° C., between about −80° C. and about −100° C., between about −80° C. and about −90° C., between about −80° C. and about −85° C., between about −120° C. and about −200° C., between about −120° C. and about −190° C., between about −120° C. and about −180° C., between about −120° C. and about −170° C., between about −120° C. and about −160° C., between about −120° C. and about −150° C., between about −120° C. and about −140° C., between about −120° C. and about −130° C., between about −120° C. and about −125° C., between about −180° C. and about −200° C., or between about −190° C. and about −200° C.). In at least some instances, the biofilm composition can be frozen by subjecting the biofilm composition to a temperature of about −80° C.

Temperature change rate during the freezing step can affect crystal size, which can impact sublimation rates during lyophilization. In any of the methods to prepare a dehydrated biofilm composition provided herein, the temperature change rate during the freezing step can be between about 0.1° C. per minute and about 10° C. per minute (e.g., between about 0.1° C. per minute and about 9° C. per minute, between about 0.1° C. per minute and about 8° C. per minute, between about 0.1° C. per minute and about 7° C. per minute, between about 0.1° C. per minute and about 6° C. per minute, between about 0.1° C. per minute and about 5° C. per minute, between about 0.1° C. per minute and about 4° C. per minute, between about 0.1° C. per minute and about 3° C. per minute, between about 0.1° C. per minute and about 2° C. per minute, between about 0.1° C. per minute and about 1° C. per minute, between about 0.1° C. per minute and about 0.9° C. per minute, between about 0.1° C. per minute and about 0.8° C. per minute, between about 0.1° C. per minute and about 0.7° C. per minute, between about 0.1° C. per minute and about 0.6° C. per minute, between about 0.1° C. per minute and about 0.5° C. per minute, between about 0.1° C. per minute and about 0.4° C. per minute, between about 0.1° C. per minute and about 0.3° C. per minute, between about 0.1° C. per minute and about 0.2° C. per minute, between about 0.5° C. per minute and about 10° C. per minute, between about 0.5° C. per minute and about 9° C. per minute, between about 0.5° C. per minute and about 8° C. per minute, between about 0.5° C. per minute and about 7° C. per minute, between about 0.5° C. per minute and about 6° C. per minute, between about 0.5° C. per minute and about 5° C. per minute, between about 0.5° C. per minute and about 4° C. per minute, between about 0.5° C. per minute and about 3° C. per minute, between about 0.5° C. per minute and about 2° C. per minute, between about 0.5° C. per minute and about 1° C. per minute, between about 0.5° C. per minute and about 0.9° C. per minute, between about 0.5° C. per minute and about 0.8° C. per minute, between about 0.5° C. per minute and about 0.7° C. per minute, between about 0.5° C. per minute and about 0.6° C. per minute, between about 1° C. per minute and about 10° C. per minute, between about 1° C. per minute and about 9° C. per minute, between about 1° C. per minute and about 8° C. per minute, between about 1° C. per minute and about 7° C. per minute, between about 1° C. per minute and about 6° C. per minute, between about 1° C. per minute and about 5° C. per minute, between about 1° C. per minute and about 4° C. per minute, between about 1° C. per minute and about 3° C. per minute, between about 1° C. per minute and about 2° C. per minute, between about 2° C. per minute and about 10° C. per minute, between about 2° C. per minute and about 9° C. per minute, between about 2° C. per minute and about 8° C. per minute, between about 2° C. per minute and about 7° C. per minute, between about 2° C. per minute and about 6° C. per minute, between about 2° C. per minute and about 5° C. per minute, between about 2° C. per minute and about 4° C. per minute, between about 2° C. per minute and about 3° C. per minute, between about 3° C. per minute and about 7° C. per minute, between about 4° C. per minute and about 6° C. per minute, between about 5° C. per minute and about 10° C. per minute, between about 5° C. per minute and about 7° C. per minute, between about 7° C. per minute and about 9° C. per minute, or between about 8° C. per minute and about 10° C. per minute). In any of the methods to prepare a dehydrated biofilm composition provided herein, the temperature of the biofilm composition can be brought to a freezing temperature at a rate of between about 1° C. per minute and about 10° C. per minute. In some embodiments, it may be desirable to use a slow rate of cooling (e.g., a rate of about 1° C. per minute or a rate of about 0.5° C. per minute). In other embodiments, an extremely rapid rate of cooling (e.g., using flash freezing) is used. In some embodiments, the biofilm composition is subjected to minor fluctuations in freezing temperature (e.g., ±0.1° C., ±0.2° C., ±0.3° C., ±0.4° C., ±0.5° C., ±0.6° C., ±0.7° C., ±0.8° C., ±0.9° C., ±1.0° C., ±1.1° C., ±1.2° C., ±1.3° C., ±1.4° C., ±1.5° C., ±1.6° C., ±1.7° C., ±1.8° C., ±1.9° C., or ±2.0° C.), e.g., that is caused by uncontrollable factors pertaining to the freezing equipment.

In some embodiments, the biofilm composition is placed in a freezer or a freeze dryer that is pre-chilled. In at least some instances, the biofilm composition is subjected to a pre-chilled freezing temperature of about −80° C.

In any of the methods to prepare a dehydrated biofilm composition provided herein, the biofilm composition can be subjected to freezing temperatures for between about 30 minutes and about 24 hours (e.g., between about 0.5 hours and 22 hours, between about 0.5 hours and 20 hours, between about 0.5 hours and 18 hours, between about 0.5 hours and 16 hours, between about 0.5 hours and 14 hours, between about 0.5 hours and 12 hours, between about 0.5 hours and 10 hours, between about 0.5 hours and 8 hours, between about 0.5 hours and 6 hours, between about 0.5 hours and 4 hours, between about 0.5 hours and 2 hours, between about 0.5 hours and 1 hours, between about 1 hour and 22 hours, between about 1 hour and 20 hours, between about 1 hour and 18 hours, between about 1 hour and 16 hours, between about 1 hour and 14 hours, between about 1 hour and 12 hours, between about 1 hour and 10 hours, between about 1 hour and 8 hours, between about 1 hour and 6 hours, between about 1 hour and 4 hours, between about 1 hour and 2 hours, between about 2 hours and 22 hours, between about 2 hours and 20 hours, between about 2 hours and 18 hours, between about 2 hours and 16 hours, between about 2 hours and 14 hours, between about 2 hours and 12 hours, between about 2 hours and 10 hours, between about 2 hours and 8 hours, between about 2 hours and 6 hours, between about 2 hours and 4 hours, or between about 2 hours and 3 hours). In some embodiments, the biofilm composition can be subjected to freezing temperatures for about less than one second or about less than ten seconds (e.g., when flash freezing the biofilm composition). In some embodiments, the biofilm composition can be subjected to freezing temperatures for more than 24 hours. In at least some instances, the biofilm composition can be subjected to freezing temperatures for between about 1 hour and about 3 hours. In some embodiments, the biofilm composition is frozen at −80° C. and then held at a different freezing temperature (e.g., any of the exemplary freezing temperatures described herein) for a duration of time (e.g., about 0.5 hours, about 1 hour, about 1.5 hours, about 2 hours, about 2.5 hours, about 3 hours, about 4 hours, about 5 hours, or more than 5 hours) prior to the primary drying phase.

There are several types and/or styles of freeze dryers. A freeze dryer capable of dehydrating a biofilm composition (e.g., any of the exemplary biofilm compositions described herein) by sublimation can be used in any of the methods of preparing a dehydrated biofilm composition provided herein. In some embodiments, a manifold freeze dryer is used. In other embodiments, a shelf or tray freeze dryer is used. The primary drying phase and optional secondary drying phase can be characterized by temperature, temperature change rate, pressure, pressure change rate, and drying duration. Additional aspects for characterizing the dehydration procedure (e.g., the freezing phase, primary drying phase, and optional secondary drying phase) parameters and sample properties are contemplated. In any of the methods of preparing a dehydrated biofilm composition provided herein, the freeze dryer subjects a biofilm composition to a low pressure vacuum (e.g., a pressure below 750 mTorr, a pressure below 700 mTorr, a pressure below 650 mTorr, a pressure below 600 mTorr, a pressure below 550 mTorr, a pressure below 500 mTorr, a pressure below 450 mTorr, a pressure below 400 mTorr, a pressure below 350 mTorr, a pressure below 300 mTorr, or a pressure below 250 mTorr, or a pressure below 200 mTorr) for a sustained period of time (e.g., between about 30 minutes and about 2 hours, between about 1 hour and about 5 hours, between about 2 hours and about 24 hours, between about 4 hours and about 8 hours, between about 6 hours and about 12 hours, between about 8 hours and about 36 hours, between about 15 hours and about 30 hours, between about 16 hours and 24 hours, or between about 17 hours and 20 hours).

In any of the methods of preparing a dehydrated biofilm composition provided herein, the primary drying phase temperature can be between about −10° C. and about −80° C. In any of the methods of preparing a dehydrated biofilm composition provided herein, the primary drying phase temperature can be between about −5° C. and about −80° C. In some embodiments, the primary drying phase temperature can be between about −5° C. and about −50° C. (e.g., between about −5° C. and about −45° C., between about −5° C. and about −40° C., between about −5° C. and about −35° C., between about −5° C. and about −30° C., between about −5° C. and about −25° C., between about −5° C. and about −20° C., between about −5° C. and about −10° C., between about −10° C. and about −50° C., between about −10° C. and about −45° C., between about −10° C. and about −40° C., between about −10° C. and about −35° C., between about −10° C. and about −30° C., between about −10° C. and about −25° C., between about −10° C. and about −20° C., between about −10° C. and about −15° C., between about −15° C. and about −50° C., between about −15° C. and about −45° C., between about −15° C. and about −40° C., between about −15° C. and about −35° C., between about −15° C. and about −30° C., between about −15° C. and about −25° C., between about −15° C. and about −20° C., between about −20° C. and about −50° C., between about −20° C. and about −45° C., between about −20° C. and about −40° C., between about −20° C. and about −35° C., between about −20° C. and about −30° C., between about −20° C. and about −25° C., between about −25° C. and about −50° C., between about −25° C. and about −45° C., between about −25° C. and about −40° C., between about −25° C. and about −35° C., between about −25° C. and about −30° C., between about −30° C. and about −50° C., between about −30° C. and about −45° C., between about −30° C. and about −40° C., between about −30° C. and about −35° C., between about −35° C. and about −50° C., between about −35° C. and about −45° C., between about −35° C. and about −40° C., between about −40° C. and about −50° C., between about −40° C. and about −45° C., or between about −45° C. and about −50° C.). In some embodiments, the primary drying phase temperature is −50° C. In some embodiments, the primary drying phase temperature is about −30° C.

In any of the methods of preparing a dehydrated biofilm composition provided herein, the secondary drying phase temperature can be between about −10° C. and about 50° C. (e.g., between about −10° C. and about 45° C., between about −10° C. and about 40° C., between about −10° C. and about 35° C., between about −10° C. and about 30° C., between about −10° C. and about 25° C., between about −10° C. and about 20° C., between about −10° C. and about 10° C., between about −10° C. and about 5° C., between about −10° C. and about 0° C., between about −10° C. and about −5° C., between about −5° C. and about 50° C., between about −5° C. and about 45° C., between about −5° C. and about 40° C., between about −5° C. and about 35° C., between about −5° C. and about 30° C., between about −5° C. and about 25° C., between about −5° C. and about 20° C., between about −5° C. and about 15° C., between about −5° C. and about 10° C., between about −5° C. and about 5° C., between about −5° C. and about 0° C., between about 0° C. and about 50° C., between about 0° C. and about 45° C., between about 0° C. and about 40° C., between about 0° C. and about 35° C., between about 0° C. and about 30° C., between about 0° C. and about 25° C., between about 0° C. and about 20° C., between about 0° C. and about 15° C., between about 0° C. and about 10° C., between about 0° C. and about 5° C., between about 5° C. and about 50° C., between about 5° C. and about 45° C., between about 5° C. and about 40° C., between about 5° C. and about 35° C., between about 5° C. and about 30° C., between about 5° C. and about 25° C., between about 5° C. and about 20° C., between about 5° C. and about 15° C., between about 5° C. and about 10° C., between about 10° C. and about 50° C., between about 10° C. and about 45° C., between about 10° C. and about 40° C., between about 10° C. and about 35° C., between about 10° C. and about 30° C., between about 10° C. and about 25° C., between about 10° C. and about 20° C., between about 10° C. and about 15° C., between about 15° C. and about 50° C., between about 15° C. and about 45° C., between about 15° C. and about 40° C., between about 15° C. and about 35° C., between about 15° C. and about 30° C., between about 15° C. and about 25° C., between about 15° C. and about 20° C., between about 20° C. and about 50° C., between about 20° C. and about 45° C., between about 20° C. and about 40° C., between about 20° C. and about 35° C., between about 20° C. and about 30° C., between about 20° C. and about 25° C., between about 25° C. and about 50° C., between about 25° C. and about 45° C., between about 25° C. and about 40° C., between about 25° C. and about 35° C., between about 25° C. and about 30° C., between about 30° C. and about 50° C., between about 30° C. and about 45° C., between about 30° C. and about 40° C., between about 30° C. and about 35° C., between about 35° C. and about 50° C., between about 35° C. and about 45° C., between about 35° C. and about 40° C., between about 40° C. and about 50° C., between about 40° C. and about 45° C., or between about 45° C. and about 50° C.). In some embodiments, the secondary drying phase temperature can be between about 0° C. and about 20° C. In some embodiments, the secondary drying phase temperature is about 20° C. In other examples, the secondary drying phase temperature is about 10° C.

In any of the methods of preparing a dehydrated biofilm composition provided herein, the primary drying phase and/or secondary drying phase rate of temperature change can each be between about 0.1° C. per minute and about 10° C. per minute (e.g., between about 0.1° C. per minute and about 9° C. per minute, between about 0.1° C. per minute and about 8° C. per minute, between about 0.1° C. per minute and about 7° C. per minute, between about 0.1° C. per minute and about 6° C. per minute, between about 0.1° C. per minute and about 5° C. per minute, between about 0.1° C. per minute and about 4° C. per minute, between about 0.1° C. per minute and about 3° C. per minute, between about 0.1° C. per minute and about 2° C. per minute, between about 0.1° C. per minute and about 1° C. per minute, between about 0.1° C. per minute and about 0.5° C. per minute, between about 0.1° C. per minute and about 0.25° C. per minute, between about 0.25° C. per minute and about 10° C. per minute, between about 0.25° C. per minute and about 9° C. per minute, between about 0.25° C. per minute and about 8° C. per minute, between about 0.25° C. per minute and about 7° C. per minute, between about 0.25° C. per minute and about 6° C. per minute, between about 0.25° C. per minute and about 5° C. per minute, between about 0.25° C. per minute and about 4° C. per minute, between about 0.25° C. per minute and about 3° C. per minute, between about 0.25° C. per minute and about 2° C. per minute, between about 0.25° C. per minute and about 1° C. per minute, between about 0.25° C. per minute and about 0.75° C. per minute, between about 0.25° C. per minute and about 0.5° C. per minute, between about 0.5° C. per minute and about 10° C. per minute, between about 0.5° C. per minute and about 9° C. per minute, between about 0.5° C. per minute and about 8° C. per minute, between about 0.5° C. per minute and about 7° C. per minute, between about 0.5° C. per minute and about 6° C. per minute, between about 0.5° C. per minute and about 5° C. per minute, between about 0.5° C. per minute and about 4° C. per minute, between about 0.5° C. per minute and about 3° C. per minute, between about 0.5° C. per minute and about 2° C. per minute, between about 0.5° C. per minute and about 1° C. per minute, between about 1° C. per minute and about 10° C. per minute, between about 1° C. per minute and about 9° C. per minute, between about 1° C. per minute and about 8° C. per minute, between about 1° C. per minute and about 7° C. per minute, between about 1° C. per minute and about 6° C. per minute, between about 1° C. per minute and about 5° C. per minute, between about 1° C. per minute and about 4° C. per minute, between about 1° C. per minute and about 3° C. per minute, between about 1° C. per minute and about 2° C. per minute, between about 2° C. per minute and about 10° C. per minute, between about 2° C. per minute and about 8° C. per minute, between about 2° C. per minute and about 6° C. per minute, between about 2° C. per minute and about 4° C. per minute, between about 3° C. per minute and about 9° C. per minute, between about 3° C. per minute and about 6° C. per minute, between about 4° C. per minute and about 8° C. per minute, between about 4° C. per minute and about 6° C. per minute, between about 5° C. per minute and about 10° C. per minute, between about 5° C. per minute and about 8° C. per minute, between about 6° C. per minute and about 10° C. per minute, or between about 8° C. per minute and about 10° C. per minute). In at least some instances, the primary drying phase and/or secondary drying phase rate of temperature change can each be between about 0.5° C. per minute and about 1° C. per minute.

In some embodiments, the primary drying phase and secondary drying phase have the same pressure. In other embodiments, the primary drying phase and secondary drying phase have different pressures. In any of the methods of preparing a dehydrated biofilm composition provided herein, the primary drying phase and/or secondary drying phase pressure can each be between about 5 mTorr and about 2,000 mTorr (e.g., between about 5 mTorr and about 1,800 mTorr, between about 5 mTorr and about 1,500 mTorr, between about 5 mTorr and about 1,200 mTorr, between about 5 mTorr and about 1,100 mTorr, between about 5 mTorr and about 1,000 mTorr, between about 5 mTorr and about 950 mTorr, between about 5 mTorr and about 900 mTorr, between about 5 mTorr and about 800 mTorr, between about 5 mTorr and about 700 mTorr, between about 5 mTorr and about 600 mTorr, between about 5 mTorr and about 500 mTorr, between about 5 mTorr and about 400 mTorr, between about 5 mTorr and about 300 mTorr, between about 5 mTorr and about 200 mTorr, between about 5 mTorr and about 100 mTorr, between about 5 mTorr and about 50 mTorr, between about 45 mTorr and about 950 mTorr, between about 45 mTorr and about 900 mTorr, between about 45 mTorr and about 800 mTorr, between about 45 mTorr and about 700 mTorr, between about 45 mTorr and about 600 mTorr, between about 45 mTorr and about 500 mTorr, between about 45 mTorr and about 400 mTorr, between about 45 mTorr and about 300 mTorr, between about 45 mTorr and about 200 mTorr, between about 45 mTorr and about 100 mTorr, between about 45 mTorr and about 55 mTorr, between about 100 mTorr and about 900 mTorr, between about 100 mTorr and about 700 mTorr, between about 100 mTorr and about 500 mTorr, between about 100 mTorr and about 400 mTorr, between about 100 mTorr and about 300 mTorr, between about 100 mTorr and about 200 mTorr, between about 100 mTorr and about 150 mTorr, between about 150 mTorr and about 350 mTorr, between about 150 mTorr and about 250 mTorr, between about 45 mTorr and about 55 mTorr, between about 200 mTorr and about 800 mTorr, between about 200 mTorr and about 700 mTorr, between about 200 mTorr and about 600 mTorr, between about 200 mTorr and about 500 mTorr, between about 200 mTorr and about 400 mTorr, between about 200 mTorr and about 300 mTorr, between about 300 mTorr and about 950 mTorr, between about 300 mTorr and about 900 mTorr, between about 300 mTorr and about 800 mTorr, between about 300 mTorr and about 700 mTorr, between about 300 mTorr and about 600 mTorr, between about 300 mTorr and about 500 mTorr, between about 300 mTorr and about 400 mTorr, between about 400 mTorr and about 950 mTorr, between about 400 mTorr and about 900 mTorr, between about 400 mTorr and about 800 mTorr, between about 400 mTorr and about 700 mTorr, between about 400 mTorr and about 600 mTorr, between about 400 mTorr and about 500 mTorr, between about 500 mTorr and about 950 mTorr, between about 500 mTorr and about 900 mTorr, between about 500 mTorr and about 800 mTorr, between about 500 mTorr and about 700 mTorr, between about 500 mTorr and about 600 mTorr, between about 600 mTorr and about 950 mTorr, between about 600 mTorr and about 900 mTorr, between about 600 mTorr and about 800 mTorr, or between about 600 mTorr and about 700 mTorr). In any of the methods of preparing a dehydrated biofilm composition provided herein, the drying phase pressure (e.g., the pressure of the primary drying phase or secondary drying phase) can be less than 200 mTorr. In some embodiments, the drying phase pressure is 200 mTorr.

In any of the methods of preparing a dehydrated biofilm composition provided herein, the primary drying phase and/or secondary drying phase can each have a duration of between about 0.5 hours and about 72 hours (e.g., between about 0.5 hours and about 70 hours, between about 0.5 hours and about 48 hours, between about 0.5 hours and about 36 hours, between about 0.5 hours and about 32 hours, between about 0.5 hours and about 24 hours, between about 0.5 hours and about 18 hours, between about 0.5 hours and about 12 hours, between about 0.5 hours and about 8 hours, between about 0.5 hours and about 4 hours, between about 1 hour and about 36 hours, between about 1 hour and about 24 hours, between about 1 hour and about 18 hours, between about 1 hour and about 12 hours, between about 1 hour and about 8 hours, between about 1 hour and about 4 hours, between about 2 hours and about 36 hours, between about 2 hours and about 24 hours, between about 2 hours and about 18 hours, between about 2 hours and about 12 hours, between about 2 hours and about 8 hours, between about 2 hours and about 4 hours, between about 3 hours and about 4 hours, between about 4 hours and about 36 hours, between about 4 hours and about 24 hours, between about 4 hours and about 18 hours, between about 4 hours and about 12 hours, between about 4 hours and about 8 hours, between about 4 hours and about 6 hours, between about 5 hours and about 10 hours, between about 5 hours and about 6 hours, between about 6 hours and about 12 hours, between about 8 hours and about 16 hours, between about 8 hours and about 12 hours, between about 10 hours and about 14 hours, between about 11 hours and about 16 hours, between about 12 hours and about 24 hours, between about 16 hours and about 20 hours, between about 18 hours and about 24 hours, between about 19 hours and about 21 hours, between about 20 hours and about 22 hours, between about 23 hours and about 25 hours, between about 28 hours and about 32 hours, or between about 32 hours and about 36 hours). In some embodiments, the primary drying phase has a duration of about 16 hours. In some embodiments, the primary drying phase is between about 11 hours and about 13 hours. In some embodiments, the primary drying phase is about 12 hours. In any of the methods of preparing a dehydrated biofilm composition provided herein, the secondary drying phase can have a duration of between about 2 hour and about 8 hours. In some embodiments, the secondary drying phase has a duration of about 6 hours.

In some embodiments, the primary drying phase and/or secondary drying phase can each comprise more than one step (e.g., two steps, three steps, or more than three steps) wherein the temperature, temperature change rate, pressure, pressure change rate, and/or duration vary. For example, the temperature and/or pressure of the primary drying phase and/or secondary drying phase can be set at a certain value (e.g., any of the exemplary temperatures or pressures described herein) for a duration of time (e.g., any of the exemplary durations of time described herein) and then the temperature and/or pressure can then be changed to a different value for a duration of time. For example, in some embodiments, the primary drying phase has a first step wherein the temperature is about −35° C. for about 5 hours and a second step wherein the temperature is about −25° C. for about 3 hours. In some embodiments, the secondary drying phase has a first step wherein the temperature is about 5° C. for about 6 hours and a second step wherein the temperature is about 15° C. for about 4 hours.

In some embodiments, after the final drying phase the dehydrated biofilm composition is optionally subjected to a hold step in the lyophilizer wherein the dehydrated biofilm composition is subjected to a temperature between about 2° C. and about 7° C. (e.g., between about 2° C. and about 4° C., between about 2° C. and about 6° C., between about 2° C. and about 5° C., between about 4° C. and about 6° C., between about 4° C. and about 7° C., between about 5° C. and about 7° C., between about 4° C. and about 5° C., or between about 3.5° C. and about 5.5° C.).

After the completion of the drying phases of the dehydration process, it is desired to prevent any moisture and oxygen from interacting with the dehydrated biofilm composition. In some embodiments, the dehydrated biofilm composition is enclosed within a container (e.g., any of the containers described herein) in order to prevent any moisture and oxygen from entering the dehydrated biofilm composition. As can be appreciated by those skilled in the art, a variety of procedures can be used to seal the container comprising the dehydrated biofilm composition.

The dehydrated biofilm composition can be stored at a temperature between about 2° C. and about 25° C. In at least some instances, the dehydrated biofilm composition is stored at a temperature between about 2° C. and about 7° C. (e.g., between about 2° C. and about 6° C., between about 2° C. and about 5° C., between about 2° C. and about 4° C., between about 3° C. and about 6° C., between about 3° C. and about 5° C., between about 4° C. and about 5° C., between about 4° C. and about 6° C., or between about 5° C. and about 6° C.). In some embodiments, the dehydrated biofilm composition can then be stored at about room temperature (e.g., between about 20° C. and about 25° C., between about 20° C. and about 24° C., between about 20° C. and about 23° C., between about 20° C. and about 22° C., between about 20° C. and about 21° C., between about 21° C. and about 25° C., between about 21° C. and about 24° C., between about 21° C. and about 23° C., between about 21° C. and about 22° C., between about 22° C. and about 25° C., between about 22° C. and about 24° C., between about 22° C. and about 23° C., between about 23° C. and about 25° C., between about 23° C. and about 24° C., between about 24° C. and about 25° C., or between about 22.5° C. and about 23.5° C.).

The steps and parameters used in any of the methods of preparing a dehydrated biofilm composition provided herein can be determined based, at least in part, on the characteristics of the biofilm composition used in a particular instance of any of the methods. For example, the microbial organism(s), the quantity of viable microbes, the stabilizing agent(s), or the concentration of stabilizing agent(s) in a particular biofilm composition can impact the optimal parameters for preparing a dehydrated biofilm composition.

In some embodiments, it is desired to determine the optimal parameters for preparing a particular dehydrated biofilm composition (e.g., any of the biofilm compositions described herein) experimentally or empirically. In other embodiments, the type of freeze dryer used can impact the optimal parameters for preparing a particular dehydrated biofilm composition. As can be appreciated by those skilled in the art, the methods described herein can be utilized to experiment and determine optimal conditions for a particular biofilm composition.

Methods of Rehydrating Dehydrated Biofilm Compositions

Also provided herein are methods of rehydrating a dehydrated biofilm composition (e.g., any of the dehydrated biofilm compositions provided herein) that comprise submerging the dehydrated biofilm composition in an aqueous solution and maintaining the dehydrated biofilm composition in the aqueous solution for an amount of time sufficient for reconstitution. Upon reconstitution, any of the dehydrated biofilm compositions provided herein remain attached to a substrate, have a viable cell count, and exhibit a biofilm phenotype. By using any of the methods of rehydrating dehydrated biofilm compositions described herein, the reconstituted biofilm compositions are ready for use, e.g., in screening the anti-biofilm activity of a test substance.

Also provided herein are methods of rehydrating a dehydrated biofilm composition (e.g., any of the dehydrated biofilm compositions provided herein) that comprise pre-hydrating the dehydrated biofilm composition, then submerging the dehydrated biofilm composition in an aqueous solution and maintaining the dehydrated biofilm composition in the aqueous solution for an amount of time sufficient for reconstitution. In any of the methods provided herein, pre-hydration involves exposing a dehydrated biofilm composition to water saturated air at a specific temperature and for a set amount of time. Pre-hydration can improve the viability or integrity of the dehydrated biofilm composition upon reconstitution. For example, pre-hydration can decrease the extent of adverse effects experienced by the dehydrated biofilm composition during rehydration, e.g., alterations of morphology.

Any of the methods of rehydrating dehydrated biofilm compositions described herein can further include one or more (e.g., two, three, four, five or more than five) washing steps, e.g., with an aqueous solution. One or more washing steps can be used, e.g., to remove lyoprotectant, cryoprotectant, or unattached cells. Any of the methods can also further include agitating the dehydrated biofilm composition during one or more steps in the rehydration process, e.g., by placing the composition on a shaker.

The aqueous solution included in any of these methods can comprise water and, optionally, further comprise additional substances, e.g., excipient(s) or nutrient(s). Excipient(s) can be included, e.g., to improve rehydration efficiency or for use in an assay methodology. Nutrient(s) can be included, e.g., to enable biofilm growth after reconstitution. The composition and properties of the aqueous solution can impact the viability and integrity of the dehydrated biofilm composition during the rehydration process. It may be desirable to have an aqueous solution with a composition and properties that minimize any potential damage. For example, osmolarity, pH, temperature, and other properties of the aqueous solution can impact the viability and integrity of the biofilm composition upon rehydration.

In some embodiments, the aqueous solution is a buffered solution. In some examples, the aqueous solution comprises one or more of the following buffering agents: citric acid, acetic acid, N-cyclohexyl-2-aminoethanesulfonic acid, monopotassium phosphate, monosodium phosphate, disodium phosphate, histidine, glutamate, [Tris(hydroxymethyl)methylamino]propanesulfonic acid (TAPS), 2-(Bis(2-hydroxyethyl)amino)acetic acid (Bicine), Tris(hydroxymethyl)aminomethane (Tris), 3-[N-Tris(hydroxymethyl)methylamino]-2-hydroxypropanesulfonic acid (Tricine), 3-[N-Tris(hydroxymethyl)methylamino]-2-hydroxypropanesulfonic acid (TAPS 0), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), Piperazine-N,N′-bis(2-ethanesulfonic acid) (PIPES), Dimethylarsenic acid (Cacodylate), 2-(N-morpholino)ethanesulfonic acid (MES), and a salt thereof. In some embodiments, the aqueous solution is phosphate buffered saline. In other embodiments, the aqueous solution is a Tris-HCl buffered solution. Other buffering agents that can be included in the aqueous solution may be utilized. Concentrations of buffering agents in solution and methods for preparing buffered solutions may also be utilized.

In some embodiments, the aqueous solution comprises one or more nutrients (e.g., a substance that can act as a nutrient to any of the microbial organisms provided herein). In some embodiments, the aqueous solution is a growth media (e.g., a culture media, a minimal media, a selective media, a differential media, a transport media, or an indicator media). In some examples, the aqueous solution is a growth media selected from the following group: nutrient broth, remel letheen broth, lysogeny broth, trypticase soy broth, terrific broth, M9 minimal broth, mannitol salt broth, eosin methylene blue broth, YM broth, MacConkey broth, and hektoen enteric broth. Additional examples of growth media and methods of preparing said growth media may be utilized. In some examples, the aqueous solution is a diluted growth media. In some examples, the aqueous solution is a growth media and further comprises a buffering agent.

In some embodiments, the aqueous solution can have a pH between about pH 4 to about pH 10 (e.g., between about pH 4 to about pH 9, between about pH 4 to about pH 8, between about pH 4 to about pH 7, between about pH 4 to about pH 6, between about pH 4 to about pH 5, between about pH 5 to about pH 10, between about pH 5 to about pH 9, between about pH 5 to about pH 8, between about pH 5 to about pH 7, between about pH 5 to about pH 6, between about pH 6 to about pH 10, between about pH 6 to about pH 9, between about pH 6 to about pH 8, between about pH 6 to about pH 7, between about pH 7 to about pH 10, between about pH 7 to about pH 9, between about pH 7 to about pH 8, between about pH 4.5 to about pH 9.5, between about pH 5.5 to about pH 8.5, between about pH 6.5 to about pH 7.5, between about pH 6 to about pH 6.5, between about pH 7 to about pH 7.5, between about pH 6.5 to about pH 7, or between about pH 7.5 to about pH 8).

As can be appreciated by those skilled in the art, a particular aqueous solution composition may be desirable depending on the particular dehydrated biofilm composition and the intended use of the dehydrated biofilm composition upon rehydration.

In any of the embodiments described herein, the dehydrated biofilm composition is rehydrated or reconstituted after being submerged and maintained in an aqueous solution for a sufficient amount of time. In some examples, the dehydrated biofilm composition is submerged in an aqueous solution and maintained in the aqueous solution for at least about 0.25 seconds (e.g., at least about 0.5 seconds, at least about 0.75 seconds, at least about 1 second, at least about 5 seconds, at least about 10 seconds, at least about 30 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 30 minutes, at least about 45 minutes, at least about 60 minutes, at least about 75 minutes, at least about 90 minutes, at least about 120 minutes, at least about 150 minutes, at least about 180 minutes, at least about 210 minutes, or at least about 240 minutes).

In some examples, the dehydrated biofilm composition is submerged in an aqueous solution and maintained in the aqueous solution for between about 0.25 seconds and about 60 seconds (e.g., between about 0.25 seconds and about 0.5 seconds, between about 0.25 seconds and about 1 second, between about 0.5 seconds and about 0.75 seconds, between about 0.5 seconds and about 1 second, between about 1 second and about 2 seconds, between about 1 second and about 3 seconds, between about 3 seconds and about 5 seconds, between about 5 seconds and about 10 seconds, between about 10 seconds and about 15 seconds, between about 15 seconds and about 20 seconds, between about 20 seconds and about 25 seconds, between about 25 seconds and about 30 seconds, between about 30 seconds and about 35 seconds, between about 35 seconds and about 40 seconds, between about 40 seconds and about 45 seconds, between about 45 seconds and about 50 seconds, between about 50 seconds and about 55 seconds, or between about 55 seconds and about 60 seconds).

In some examples, the dehydrated biofilm composition is submerged in an aqueous solution and maintained in the aqueous solution for between about 1 minute and about 240 minutes (e.g., between about 1 minute and 180 minutes, between about 1 minute and 150 minutes, between about 1 minute and 120 minutes, between about 1 minute and 90 minutes, between about 1 minute and 60 minutes, between about 1 minute and 45 minutes, between about 1 minute and 40 minutes, between about 1 minute and 35 minutes, between about 1 minute and 30 minutes, between about 1 minute and 25 minutes, between about 1 minute and 20 minutes, between about 1 minute and 18 minutes, between about 1 minute and 16 minutes, between about 1 minute and 15 minutes, between about 1 minute and 14 minutes, between about 1 minute and 12 minutes, between about 1 minute and 10 minutes, between about 1 minute and 8 minutes, between about 1 minute and 6 minutes, between about 1 minute and 5 minutes, between about 3 minutes and 15 minutes, between about 5 minutes and 60 minutes, between about 5 minutes and 55 minutes, between about 5 minutes and 50 minutes, between about 5 minutes and 45 minutes, between about 5 minutes and 40 minutes, between about 5 minutes and 35 minutes, between about 5 minutes and 30 minutes, between about 5 minutes and 25 minutes, between about 5 minutes and 20 minutes, between about 5 minutes and 15 minutes, between about 5 minutes and 10 minutes, between about 6 minutes and 40 minutes, between about 6 minutes and 36 minutes, between about 6 minutes and 30 minutes, between about 6 minutes and 24 minutes, between about 6 minutes and 18 minutes, between about 6 minutes and 12 minutes, between about 10 minutes and 30 minutes, between about 10 minutes and 25 minutes, between about 10 minutes and 20 minutes, between about 10 minutes and 15 minutes, between about 15 minutes and 60 minutes, between about 15 minutes and 55 minutes, between about 15 minutes and 50 minutes, between about 15 minutes and 45 minutes, between about 15 minutes and 40 minutes, between about 15 minutes and 35 minutes, between about 15 minutes and 30 minutes, between about 15 minutes and 25 minutes, between about 15 minutes and 20 minutes, between about 30 minutes and 60 minutes, between about 30 minutes and 55 minutes, between about 30 minutes and 50 minutes, between about 30 minutes and 45 minutes, between about 30 minutes and 40 minutes, between about 30 minutes and 35 minutes, between about 35 minutes and 45 minutes, between about 35 minutes and 40 minutes, between about 45 minutes and 60 minutes, between about 45 minutes and 55 minutes, between about 45 minutes and 50 minutes, between about 60 minutes and 240 minutes, between about 60 minutes and 210 minutes, between about 60 minutes and 180 minutes, between about 60 minutes and 150 minutes, between about 60 minutes and 120 minutes, between about 60 minutes and 90 minutes, between about 60 minutes and 75 minutes, between about 90 minutes and 240 minutes, between about 90 minutes and 210 minutes, between about 90 minutes and 180 minutes, between about 90 minutes and 150 minutes, between about 90 minutes and 120 minutes, between about 90 minutes and 105 minutes, between about 120 minutes and 240 minutes, between about 120 minutes and 180 minutes, between about 180 minutes and 240 minutes, between about 180 minutes and 210 minutes, or between about 210 minutes and 240 minutes). In some embodiments, the dehydrated biofilm composition is submerged in an aqueous solution and maintained in the aqueous solution for between about 10 minutes and 20 minutes. In at least some instances, the dehydrated biofilm composition is submerged in an aqueous solution and maintained in the aqueous solution for about 10 minutes. In at least some instances, the dehydrated biofilm composition is submerged in an aqueous solution and maintained in the aqueous solution for about 15 minutes. In at least some instances, the dehydrated biofilm composition is submerged in an aqueous solution and maintained in the aqueous solution for about 20 minutes.

As can be appreciated by those skilled in the art, the methods described herein can be utilized to experiment and determine optimal conditions for a particular dehydrated bio film composition.

Methods of Determining Anti-Biofilm Efficacy

In some situations, it is desired to test the anti-biofilm efficacy of a test substance against a rehydrated biofilm composition. Provided herein are methods of determining the anti-biofilm efficacy of a test substance that include (1) submerging a dehydrated biofilm composition in an aqueous solution, (2) maintaining the biofilm composition in the aqueous solution for an amount of time sufficient for rehydration, (3) contacting the biofilm composition with a test substance for a contact time, and (4) performing an assay to determine the anti-biofilm efficacy of the test substance.

In any of the methods of determining the anti-biofilm efficacy of a test substance provided herein, the dehydrated biofilm composition can be rehydrated using any of the methods of rehydrating a dehydrated biofilm composition described herein.

In any of the methods of determining the anti-biofilm efficacy of a test substance provided herein, the rehydrated biofilm composition can be contacted with at least one (e.g., at least two, at least three, at least four, at least five, or at least six) substance(s). In any of these methods, the biofilm composition can be contacted with a test substance within about 1 minute, about 2 minutes, about 3 minutes, about 4 minutes, about 5 minutes, about 6 minutes, about 7 minutes, about 8 minutes, about 9 minutes, about 10 minutes, about 12 minutes, about 14 minutes, about 16 minutes, about 18 minutes, about 20 minutes, about 25 minutes, about 30 minutes, about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 4 hours, about 5 hours, about 6 hours, about 8 hours, about 10 hours, about 12 hours, about 18 hours, about 24 hours, about 30 hours, about 36 hours, about 42 hours, about 48 hours, or more than 48 hours of submerging the dehydrated biofilm composition in an aqueous solution.

In any of these methods, the contact time can be about 1 minute, about 2 minutes, about 3 minutes, about 4 minutes, about 5 minutes, about 6 minutes, about 7 minutes, about 8 minutes, about 9 minutes, about 10 minutes, about 12 minutes, about 14 minutes, about 16 minutes, about 18 minutes, about 20 minutes, about 25 minutes, about 30 minutes, about 35 minutes, about 40 minutes, about 45 minutes, about 50 minutes, about 55 minutes, about 1 hour, about 1.25 hours, about 1.5 hours, about 1.75 hours, about 2 hours, about 2.25 hours, about 2.5 hours, about 2.75 hours, about 3 hours, about 4 hours, about 5 hours, about 6 hours, about 8 hours, about 10 hours, about 12 hours, about 18 hours, about 24 hours, about 30 hours, about 36 hours, about 42 hours, about 48 hours, or more than 48 hours. In any of the methods of determining the anti-biofilm efficacy of a test substance provided herein, the test substance can be formulated as a solution, a liquid, a towelette, a wipe, a spray, a powder, a gel, or a paste.

In some situations, it is desired to neutralize the substance being tested for anti-biofilm efficacy, e.g., in order to prevent additional anti-biofilm or anti-microbial activity after the contact time. In any of these methods, the biofilm composition can optionally be contacted with a neutralizer before measuring the anti-biofilm efficacy results of the test substance. An appropriate neutralizer can be selected based on the test substance being tested. Non-limiting examples of neutralizers include, e.g., dilution with water, Tween 80, sodium hydrogen sulfite, sodium thio sulfate, glycine, histidine, lecithin, pyruvate, catalase, Dey-Engley Neutralizing Broth, a buffer (e.g., phosphate buffer), or an enzyme (e.g., beta-lactamase). Additional neutralizers are contemplated.

In any of the methods of determining the anti-biofilm efficacy of a test substance provided herein, performing an assay to determine the anti-biofilm efficacy of the test substance can include qualitatively assessing or quantitatively measuring the presence, amount, or functional activity of a target entity or biofilm characteristic. The assay used to determine anti-biofilm efficacy can measure the ability of a test substance to inhibit or kill microbes in a biofilm composition, the ability to remove or detach a biofilm composition from a substrate, and/or the ability to breakdown the extracellular matrix of a biofilm composition. The assay can measure a variety of target entities or biofilm characteristics including, e.g., biofilm mass, viability, metabolism, matrix composition, adhesion extent, and adhesion strength. The assay can measure a target entity or a biofilm characteristic according to any of the methods or techniques described herein. In some embodiments, the dehydration biofilm composition is contacted by a compound (e.g., a dye or a stain) as part of an assay to determine the anti-biofilm efficacy of a test substance. A non-limiting example of an assay is the Single Tube Method (see, e.g., ASTM E2871 or EPA MLB SOP MB-20) wherein the anti-biofilm efficacy of a test substance is quantitatively measured, e.g., by CFU enumeration or crystal violet dye staining. Other assays may be used. In some examples, the assay is used to determine the minimum biofilm eradication concentration (MBEC) or minimum biofilm inhibitory concentration (MBIC) of a test substance.

Dehydrated Biofilm Arrays

Also provided herein are dehydrated biofilm arrays comprising a plurality of reservoir compartments arranged into an N×N array, wherein one or more reservoirs contains a dehydrated biofilm composition comprising a microbial cell population enmeshed in a self-produced extracellular polymeric matrix and attached to a substrate, wherein the composition retains substrate attachment and exhibits a viable cell count and a biofilm phenotype upon rehydration. The dehydrated biofilm composition can be any of the dehydrated biofilm compositions provided herein. The dehydrated biofilm arrays provided herein are advantageous in that they enable fast and efficient testing of anti-biofilm activity, including, e.g., biofilm eradication, inhibition, or removal. The dehydrated biofilm composition(s) contained within the reservoir(s) can be rehydrated and used, e.g., in a biofilm eradication assay, directly in the reservoir. The reservoir compartments in any of the arrays provided herein can be enclosed in a container or otherwise sealed from atmospheric exposure, e.g., in order to prevent degradation of the dehydrated biofilm compositions.

In some examples, any of the reservoir compartments can have a volume of between about 1 uL and about 50 mL (e.g., between about 1 uL and about 1,000 uL, between about 5 uL and about 1,000 uL, between about 5 uL and about 900 uL, between about 5 uL and about 800 uL, between about 5 uL and about 700 uL, between about 5 uL and about 600 uL, between about 5 uL and about 500 uL, between about 5 uL and about 400 uL, between about 5 uL and about 300 uL, between about 5 uL and about 200 uL, between about 5 uL and about 100 uL, between about 5 uL and about 50 uL, between about 5 uL and about 25 uL, between about 50 uL and about 1,000 uL, between about 50 uL and about 800 uL, between about 50 uL and about 600 uL, between about 50 uL and about 400 uL, between about 50 uL and about 200 uL, between about 50 uL and about 100 uL, between about 100 uL and about 1,000 uL, between about 100 uL and about 800 uL, between about 100 uL and about 600 uL, between about 100 uL and about 400 uL, between about 100 uL and about 200 uL, between about 100 uL and about 150 uL, between about 200 uL and about 1,000 uL, between about 200 uL and about 800 uL, between about 200 uL and about 600 uL, between about 200 uL and about 400 uL, between about 200 uL and about 300 uL, between about 300 uL and about 1,000 uL, between about 300 uL and about 800 uL, between about 300 uL and about 600 uL, between about 300 uL and about 400 uL, between about 500 uL and about 1,000 uL, between about 500 uL and about 900 uL, between about 500 uL and about 800 uL, between about 500 uL and about 700 uL, between about 500 uL and about 600 uL, between about 750 uL and about 1,000 uL, between about 750 uL and about 950 uL, between about 750 uL and about 900 uL, between about 750 uL and about 850 uL, between about 750 uL and about 800 uL, between about 1 mL and about 50 mL, between about 1 mL and about 45 mL, between about 1 mL and about 40 mL, between about 1 mL and about 35 mL, between about 1 mL and about 30 mL, between about 1 mL and about 25 mL, between about 1 mL and about 20 mL, between about 1 mL and about 15 mL, between about 1 mL and about 10 mL, between about 1 mL and about 9 mL, between about 1 mL and about 8 mL, between about 1 mL and about 7 mL, between about 1 mL and about 6 mL, between about 1 mL and about 5 mL, between about 1 mL and about 4 mL, between about 1 mL and about 3 mL, between about 1 mL and about 2 mL, between about 1 mL and about 1.5 mL, between about 2 mL and about 20 mL, between about 2 mL and about 18 mL, between about 2 mL and about 16 mL, between about 2 mL and about 14 mL, between about 2 mL and about 12 mL, between about 2 mL and about 10 mL, between about 2 mL and about 8 mL, between about 2 mL and about 6 mL, between about 2 mL and about 4 mL, between about 2 mL and about 3 mL, between about 5 mL and about 20 mL, between about 5 mL and about 15 mL, between about 5 mL and about 10 mL, between about 10 mL and about 25 mL, between about 10 mL and about 20 mL, between about 10 mL and about 15 mL, between about 10 mL and about 12.5 mL, between about 15 mL and about 25 mL, between about 15 mL and about 20 mL, between about 20 mL and about 30 mL, between about 20 mL and about 25 mL, between about 25 mL and about 35 mL, between about 25 mL and about 30 mL, between about 30 mL and about 40 mL, between about 30 mL and about 35 mL, between about 35 mL and about 45 mL, between about 35 mL and about 40 mL, between about 40 mL and about 50 mL, between about 40 mL and about 45 mL, between about 45 mL and about 50 mL). In some examples, the reservoir compartments have a volume of between about 300 uL and about 500 uL. In some examples, the reservoir compartments have a volume of between about 75 uL and about 200 uL. In some examples, the reservoir compartments have a volume of between about 4 uL and about 20 uL. In some examples, the reservoir compartments have a volume of about 50 mL. In any of the embodiments provided herein, the working volume of a reservoir compartment can be equal to or less than the total volume of the reservoir compartment.

The reservoir compartments in any of the arrays described herein can have a different diameter at the top of the compartment than at the bottom of the compartment. In some examples, the reservoir compartment has a curvature at the bottom of the compartment. In some examples, the reservoir compartment has a flat-bottom shape or a v-shaped bottom shape. Other reservoir compartment shapes may be utilized. In some examples, any of the reservoir compartments can have a top diameter of between about 0.5 mm and about 30 mm (e.g., between about 0.5 mm and about 25 mm, between about 0.5 mm and about 20 mm, between about 0.5 mm and about 15 mm, between about 0.5 mm and about 10 mm, between about 0.5 mm and about 5 mm, between about 0.5 mm and about 4 mm, between about 0.5 mm and about 3 mm, between about 0.5 mm and about 2 mm, between about 0.5 mm and about 1 mm, between about 0.5 mm and about 0.9 mm, between about 0.5 mm and about 0.8 mm, between about 0.5 mm and about 0.7 mm, between about 0.5 mm and about 0.6 mm, between about 1 mm and about 30 mm, between about 1 mm and about 25 mm, between about 1 mm and about 20 mm, between about 1 mm and about 15 mm, between about 1 mm and about 10 mm, between about 1 mm and about 5 mm, between about 1 mm and about 4 mm, between about 1 mm and about 3 mm, between about 1 mm and about 2 mm, between about 1 mm and about 1.5 mm, between about 2 mm and about 10 mm, between about 2 mm and about 9 mm, between about 2 mm and about 8 mm, between about 2 mm and about 7 mm, between about 2 mm and about 6 mm, between about 2 mm and about 5 mm, between about 2 mm and about 4 mm, between about 2 mm and about 3 mm, between about 2 mm and about 2.5 mm, between about 5 mm and about 15 mm, between about 5 mm and about 10 mm, between about 5 mm and about 9 mm, between about 5 mm and about 8 mm, between about 5 mm and about 7 mm, between about 5 mm and about 6 mm, between about 10 mm and about 30 mm, between about 10 mm and about 25 mm, between about 10 mm and about 20 mm, between about 10 mm and about 15 mm, between about 10 mm and about 12.5 mm, between about 15 mm and about 30 mm, between about 15 mm and about 25 mm, between about 15 mm and about 20 mm, between about 15 mm and about 17.5 mm, between about 20 mm and about 30 mm, between about 20 mm and about 25 mm, between about 20 mm and about 22.5 mm, between about 25 mm and about 30 mm, or between about 25 mm and about 27.5 mm). In some examples, the reservoir compartments have a diameter of between about 1 mm and about 2 mm. In some examples, the reservoir compartments have a diameter of between about 3 mm and about 4 mm. In some examples, the reservoir compartments have a diameter of between about 6 mm and about 7 mm.

In some examples, any of the reservoir compartments can have a depth of between about 1 mm and about 100 mm (e.g., between about 1 mm and about 90 mm, between about 1 mm and about 80 mm, between about 1 mm and about 70 mm, between about 1 mm and about 60 mm, between about 1 mm and about 50 mm, between about 1 mm and about 40 mm, between about 1 mm and about 30 mm, between about 1 mm and about 20 mm, between about 1 mm and about 15 mm, between about 1 mm and about 10 mm, between about 1 mm and about 5 mm, between about 1 mm and about 3 mm, between about 5 mm and about 100 mm, between about 5 mm and about 75 mm, between about 5 mm and about 50 mm, between about 5 mm and about 25 mm, between about 5 mm and about 20 mm, between about 5 mm and about 15 mm, between about 5 mm and about 10 mm, between about 10 mm and about 100 mm, between about 10 mm and about 90 mm, between about 10 mm and about 80 mm, between about 10 mm and about 70 mm, between about 10 mm and about 60 mm, between about 10 mm and about 50 mm, between about 10 mm and about 40 mm, between about 10 mm and about 30 mm, between about 10 mm and about 20 mm, between about 10 mm and about 19 mm, between about 10 mm and about 18 mm, between about 10 mm and about 17 mm, between about 10 mm and about 16 mm, between about 10 mm and about 15 mm, between about 10 mm and about 14 mm, between about 10 mm and about 13 mm, between about 10 mm and about 12 mm, between about 10 mm and about 11 mm, between about 20 mm and about 50 mm, between about 20 mm and about 40 mm, between about 20 mm and about 30 mm, between about 20 mm and about 25 mm, between about 30 mm and about 60 mm, between about 30 mm and about 50 mm, between about 30 mm and about 40 mm, between about 30 mm and about 35 mm, between about 40 mm and about 80 mm, between about 40 mm and about 70 mm, between about 40 mm and about 60 mm, between about 40 mm and about 50 mm, between about 50 mm and about 100 mm, between about 50 mm and about 75 mm, between about 60 mm and about 80 mm, between about 60 mm and about 70 mm, between about 70 mm and about 80 mm, between about 80 mm and about 90 mm, between about 90 mm and about 100 mm, or between about 90 mm and about 95 mm). In some examples, the reservoir compartments have a depth of between about 10 mm and about 12 mm. In some examples, the reservoir compartments have a depth of between about 11 mm and about 13 mm. In some examples, the reservoir compartments have a depth of between about 4 mm and about 6 mm.

The reservoir compartments in any of the arrays described herein can comprise an inactive material or an active material. An inactive material is one that is non-reactive when in contact or proximity to any of the biofilm compositions described herein and does not elicit a response from any of the biofilm compositions described herein. An active material is one that is dynamic or reactive when in contact or proximity to any of the biofilm compositions, or elicits a response from any of the biofilm compositions described herein. In some examples, the reservoir compartments comprise one or more of the following materials: polystyrene, polypropylene, polycarbonate, cyclo-olefin, quartz, glass, stainless steel, and titanium dioxide. In other examples, the reservoir compartments comprise polyethylene.

Any of the reservoir compartments described herein can be further coated with a substance. The substance coating a reservoir compartment can be an active or an inactive substance. An inactive substance is one that is non-reactive when in contact or proximity to any of the biofilm compositions described herein and does not elicit a response from any of the biofilm compositions described herein. An active substance is one that is dynamic or reactive when in contact or proximity to any of the biofilm compositions, or elicits a response from any of the biofilm compositions described herein. In some examples, a reservoir compartment is coated with a chemical, a compound, a plastic, a metal, a polymer, a protein, a nucleic acid, or a cell. In some examples, the coating is a hydroxyapatite or titanium dioxide. In some examples, the coating is a eukaryotic cell (e.g., an epithelial human cell) or a bacteria cell (e.g., a bacteria found within the gastrointestinal tract of a human). In some embodiments, the reservoir compartment material or coating are intended to mimic conditions that biofilms encounter in real world conditions (e.g., conditions in a human organism, in a manufacturing facility, in a water treatment facility, or in an agricultural setting). In other embodiments, the reservoir compartment material or coating (e.g., an active material or coating) are intended to cause a response (e.g., increased attachment) from the biofilm composition. Other materials and coatings are contemplated. Other reasons for including a material or coating are contemplated. As can be appreciated by one skilled in the art, a certain material or coating can be selected depending on the specific embodiment and application.

Any of the reservoir compartments in any of the arrays described herein can be arranged into a N1×N2 matrix array, wherein N1 and N2 are integers between 1 and 10,000. In some examples, N1 and N2 are the same integer. In other examples, N1 and N2 are different integers. In some examples, N1 or N2 is an integer between about 1 and 12, between about 2 and 24, between about 3 and 36, or between about 4 and 48. In some instances, the reservoir compartments of any of the arrays described herein are arranged into a 12×8 array, a 24×16 array, or a 48×32 array. In other instances, the reservoir compartments of any of the arrays described herein are arranged into a 12×2 array, an 8×3 array, or a 6×4 array. In other instances, the reservoir compartments of any of the arrays described herein are arranged into a 4×2 array or an 8×1 array.

In some examples, the reservoir compartments in the array are connected as a single solid piece of material, e.g., wherein the reservoir compartments are wells in a microtiter plate. In other examples, the reservoir compartments in the array are not connected as a single solid piece of material, e.g., wherein the reservoir compartments are vials or test tubes held in place by a rack (e.g., a test tube rack or a vial rack).

The arrays provided herein can have a height of between about 1 mm and about 150 mm (e.g., between about 1 mm and about 140 mm, between about 1 mm and about 130 mm, between about 1 mm and about 120 mm, between about 1 mm and about 110 mm, between about 1 mm and about 100 mm, between about 1 mm and about 90 mm, between about 1 mm and about 80 mm, between about 1 mm and about 70 mm, between about 1 mm and about 60 mm, between about 1 mm and about 50 mm, between about 1 mm and about 40 mm, between about 1 mm and about 30 mm, between about 1 mm and about 20 mm, between about 1 mm and about 15 mm, between about 1 mm and about 10 mm, between about 1 mm and about 5 mm, between about 1 mm and about 3 mm, between about 5 mm and about 150 mm, between about 5 mm and about 125 mm, between about 5 mm and about 100 mm, between about 5 mm and about 75 mm, between about 5 mm and about 50 mm, between about 5 mm and about 25 mm, between about 5 mm and about 20 mm, between about 5 mm and about 15 mm, between about 5 mm and about 10 mm, between about 10 mm and about 150 mm, between about 10 mm and about 125 mm, between about 10 mm and about 100 mm, between about 10 mm and about 90 mm, between about 10 mm and about 80 mm, between about 10 mm and about 70 mm, between about 10 mm and about 60 mm, between about 10 mm and about 50 mm, between about 10 mm and about 40 mm, between about 10 mm and about 30 mm, between about 10 mm and about 20 mm, between about 10 mm and about 19 mm, between about 10 mm and about 18 mm, between about 10 mm and about 17 mm, between about 10 mm and about 16 mm, between about 10 mm and about 15 mm, between about 10 mm and about 14 mm, between about 10 mm and about 13 mm, between about 10 mm and about 12 mm, between about 10 mm and about 11 mm, between about 20 mm and about 50 mm, between about 20 mm and about 40 mm, between about 20 mm and about 30 mm, between about 20 mm and about 25 mm, between about 30 mm and about 60 mm, between about 30 mm and about 50 mm, between about 30 mm and about 40 mm, between about 30 mm and about 35 mm, between about 40 mm and about 80 mm, between about 40 mm and about 70 mm, between about 40 mm and about 60 mm, between about 40 mm and about 50 mm, between about 50 mm and about 100 mm, between about 50 mm and about 75 mm, between about 60 mm and about 80 mm, between about 60 mm and about 70 mm, between about 70 mm and about 80 mm, between about 80 mm and about 90 mm, between about 90 mm and about 100 mm, between about 90 mm and about 95 mm, between about 100 mm and about 150 mm, between about 100 mm and about 140 mm, between about 100 mm and about 130 mm, between about 100 mm and about 120 mm, between about 100 mm and about 110 mm, between about 120 mm and about 150 mm, between about 120 mm and about 140 mm, between about 120 mm and about 130 mm, between about 130 mm and about 150 mm, between about 130 mm and about 140 mm, between about 140 mm and about 150 mm, or between about 140 mm and about 145 mm).

The arrays provided herein can have a length of between about 10 mm and about 1,000 mm (e.g., between about 10 mm and about 1,000 mm, between about 50 mm and about 1,000 mm, between about 100 mm and about 1,000 mm, between about 200 mm and about 1,000 mm, between about 300 mm and about 1,000 mm, between about 400 mm and about 1,000 mm, between about 500 mm and about 1,000 mm, between about 40 mm and about 800 mm, between about 40 mm and about 600 mm, between about 40 mm and about 400 mm, between about 40 mm and about 200 mm, between about 40 mm and about 100 mm, between about 40 mm and about 60 mm, between about 100 mm and about 500 mm, between about 200 mm and about 600 mm, between about 200 mm and about 400 mm, between about 400 mm and about 600 mm, between about 600 mm and about 1,000 mm, or between about 750 mm and about 1,000 mm).

The arrays provided herein can have a width of between about 10 mm and about 1,000 mm (e.g., between about 10 mm and about 1,000 mm, between about 50 mm and about 1,000 mm, between about 100 mm and about 1,000 mm, between about 200 mm and about 1,000 mm, between about 300 mm and about 1,000 mm, between about 400 mm and about 1,000 mm, between about 500 mm and about 1,000 mm, between about 40 mm and about 800 mm, between about 40 mm and about 600 mm, between about 40 mm and about 400 mm, between about 40 mm and about 200 mm, between about 40 mm and about 100 mm, between about 40 mm and about 60 mm, between about 100 mm and about 500 mm, between about 200 mm and about 600 mm, between about 200 mm and about 400 mm, between about 400 mm and about 600 mm, between about 600 mm and about 1,000 mm, or between about 750 mm and about 1,000 mm).

Methods of Preparing and Dosing Dehydrated Biofilm Arrays

Also provided herein are methods of preparing and dosing a dehydrated biofilm array (e.g., any of the dehydrated biofilm arrays provided herein) that include providing a plurality of biofilm compositions each comprising a microbial cell population enmeshed in a self-produced extracellular polymeric matrix, containing each of the biofilm compositions in reservoir compartments organized into a N1×N2 array, coating or submerging each biofilm composition in an effective amount of a stabilizing agent, freezing each biofilm composition, subjecting each biofilm composition to a primary drying phase (e.g., dehydration by sublimation), subjecting each biofilm composition to a secondary drying phase (e.g., dehydration by desorption), rehydrating each biofilm composition in the N1×N2 array, and contacting the array with one or more substances (e.g., a compound).

The microbial cell populations can be any of the microbial cell populations provided herein. The reservoir compartments, stabilizing agent(s), and N1×N2 array(s) described in any of the methods of preparing and dosing dehydrated biofilm arrays can be any of the reservoir compartments, stabilizing agents, and N1×N2 arrays provided herein. The methods of preparing and dosing dehydrated biofilm arrays can utilize any of the methods of preparing a dehydrated biofilm composition and/or methods of rehydrating a dehydrated biofilm composition provided herein. The array can be contacted with at least one substance (e.g., a compound) after rehydration of dehydrated biofilm composition(s). In some embodiments, the array (e.g., a dehydration biofilm composition contained with the array) is contacted with at least one substances (e.g., at least two substances, at least three substances, at least four substances, at least five substances, or more than five substances) after rehydration of the dehydrated biofilm compositions. In some embodiments, the array (e.g., a dehydration biofilm composition contained with the array) is contact by at least one substance (e.g., a dye, a drug, a protein, a small molecule, or a nutrient) as part of an assay, e.g., to determine the anti-biofilm activity of a test substance. Other methods of conducting assays (e.g., anti-biofilm assays) are contemplated.

Kits

Also included herein are kits that include at least one of any of the dehydrated biofilm compositions or dehydrated biofilm arrays described herein. In some examples, a kit includes multiple (e.g., two, three, four, five, six, seven, eight, nine, ten, twelve, eighteen, twenty-four, thirty, thirty-six, forty-eight, fifty-four, sixty, seventy, eighty, ninety, ninety-six, one-hundred, or more than one-hundred) dehydrated biofilm compositions (e.g., any of the dehydrated biofilm compositions described herein). In some examples, a kit includes a dehydrated biofilm composition comprising a microbial organism and a different dehydrated biofilm composition comprising a different microbial organism. In some examples of any of the kits, the dehydrated biofilm composition(s) are enclosed within a primary container. In some examples, more than one biofilm composition is enclosed within a single primary container. In some examples, one or more primary container(s) are then sealed within a secondary container (e.g., a moisture barrier bag or a foil bag) that has low permeability to moisture and oxygen. In some embodiments, the primary container has low permeability to moisture and/or oxygen. In some embodiments, the secondary container further comprises a desiccant (e.g., any of the exemplary desiccants provided here) or an oxygen scavenger (e.g., any of the exemplary oxygen scavengers provided here).

Some examples of any of the kits provided herein further include a buffer, a dye, or a stain. For example, any of the kits described herein can include a rehydration solution (e.g., an aqueous solution for rehydrating the dehydrated biofilm compositions). Some examples further include materials and/or consumables (e.g., a 50 mL conical tube) used to conduct an assay (e.g., an assay for measuring anti-biofilm activity of a compound). Some examples include a nutrient media to support the growth of microorganisms, e.g., to plate and count colony forming units, e.g., after testing for anti-biofilm activity. Some examples of any of the kits provided herein further include an antimicrobial substance (e.g., an antimicrobial drug or a disinfectant), e.g., to be used as a part of a control sample in an assay. Any of the kits provided herein can be used to determine the anti-microbial activity of a test substance, e.g., that is formulated as a solution, a liquid, a towelette, a wipe, a spray, a powder, a gel, or a paste.

The disclosure is further described in the following examples, which do not limit the scope of the disclosure described in the claims.

### EXAMPLES

### Example 1. Dehydration and Subsequent Rehydration of a Pseudomonas aeruginosa Biofilm Composition

An experiment was conducted to investigate if a dehydrated biofilm composition comprising Pseudomonas aeruginosa biofilm could be prepared and successfully rehydrated while preserving viability, integrity of the extracellular matrix and substrate attachment, and overall biofilm phenotype.

Exemplary dehydrated biofilm compositions comprising Pseudomonas aeruginosa (ATCC 15442) attached to a glass disc substrate (12.7 mm diameter, 3.8 mm thickness) with a mean log density of 8.2 log CFU (1.6×10⁸ CFU) and further comprising 50 mg sucrose and 0.79 mg TRIS were prepared by the following method.

Preparation of Dehydration Biofilm Compositions: Twenty-four Pseudomonas aeruginosa ATCC 15442 biofilms were grown on glass coupons (12.7 mm diameter, 3.8 mm thickness) according to the standard CDC biofilm reactor method (EPA MLB SOP MB-19). The biofilm samples were then washed by transferring to each to a separate container with TRIS buffer solution (pH 7.4). The buffer solution was then removed and a filter sterilized solution of stabilizing agents comprising a sugar and a buffer (10% sucrose, 10 mM TRIS, pH 7.4) was added to each container. The samples were then frozen at −80° C. and placed in a lyophilizer. The following parameter settings were used for the lyophilization run:
1. Freeze −40° C. for 1 hour (vacuum off)
2. Primary drying: Raise temperature 0.3° C./minute to −31.7° C. Hold at −31.7° C. for 20 hours (vacuum on)
3. Secondary drying: Raise temperature 0.3° C./minute to −6.7° C. Hold at −6.7° C. for 2 hours (vacuum on)
4. Secondary drying: Raise temperature 0.3° C./minute to 10° C. Hold at 10° C. for 2 hours (vacuum on).
5. Hold step: Reduced temperature to 3.9° C. Vacuum off.

Each dehydrated biofilm composition was then removed from the vessel chamber and sealed in a 15×22 cm foil pouch with a 5 g silica desiccant. The dehydrated biofilm compositions were then stored at refrigeration temperature 4-8° C.

Rehydration of the Dehydrated Biofilm Compositions & Determination of Viable Cell Count:

After approximately 72 hours, the dehydrated biofilm compositions were removed from the foil pouch and placed into a container. The dehydrated biofilm compositions were then submerged in 2 mL of a rehydration solution (sterile DI water, pH 7.4) and maintained in the solution for 20 minutes at room temperature to allow for rehydration of the biofilm composition. Upon rehydration it was observed that the composition successfully retained its biofilm phenotype, including the integrity of the extracellular matrix and attachment to the substrate. The viability of the rehydrated biofilm composition was then evaluated by determining the CFU per composition. First each rehydrated biofilm composition was vortexed (30 seconds) and sonicated (45 kHz for approximately 30 seconds). This step was repeated twice. The sample was then serially diluted in Standard Method Dilution Water (SMDW—0.0425 g/L KH₂PO₄ and 0.405 g/L MgCl₂) and spread plated onto trypticase soy agar (TSA). The plated samples were then incubated at 37° C. for 24±4 hours. The agar plates were then counted and CFU per composition was calculated. The mean log density was calculated to be 8.2 log CFU per composition.

### Example 2. Dehydration and Subsequent Rehydration of a Staphylococcus aureus Biofilm Composition

An experiment was conducted to investigate if a dehydrated biofilm composition comprising Staphylococcus aureus biofilm could be prepared and successfully rehydrated while preserving viability, integrity of the extracellular matrix and substrate attachment, and overall biofilm phenotype.

Exemplary dehydrated biofilm compositions comprising Staphylococcus aureus ATCC 6538 attached to a K1 carrier (10×7 mm) with a mean log density of 7.7 log CFU (5×10⁷ CFU) and further comprising 50 mg sucrose and 0.79 mg TRIS were prepared by the following method.

Preparation of Dehydration Biofilm Compositions: Staphylococcus aureus ATCC 6538 biofilms were grown on K1 carriers at 37° C. with turbulent flow for at least 24 hours. The biofilm sample was then washed by transferring to a container containing TRIS buffer solution (pH 7.4). The buffer solution was then removed from the container and about 83 mg of stabilizing agents were added to each container. The samples were then frozen at −80° C. and placed in a lyophilizer. The following parameter settings were used for the lyophilization run:
1. Freeze −40° C. for 1 hour (vacuum off)
2. Primary drying: Raise temperature 0.3° C./minute to −31.7° C. Hold at −31.7° C. for 12 hours (vacuum on)
3. Secondary drying: Raise temperature 0.3° C./minute to −6.7° C. Hold at −6.7° C. for 3 hours (vacuum on)
4. Secondary drying: Raise temperature 0.3° C./minute to 10° C. Hold at 10° C. for 3 hours (vacuum on).
5. Hold step: Reduced temperature to 3.9° C. Vacuum off.

The resulting dehydrated biofilm compositions were then removed from the vessel chamber and sealed in a 15×22 cm foil pouch. The dehydrated biofilm compositions were then stored at refrigeration temperature 4-8° C.

Rehydration of the Dehydrated Biofilm Compositions & Determination of Viable Cell Count:

After approximately 72 hours, the dehydrated biofilm compositions were removed from the foil pouch and placed into a container. The dehydrated biofilm compositions were then submerged in 2 mL of a rehydration solution (TRIS buffer, pH 7.4) and maintained in the solution for 20 minutes at room temperature to allow for rehydration of the biofilm composition. Upon rehydration it was observed that the composition successfully retained its biofilm phenotype, including the integrity of the extracellular matrix and attachment to the substrate. The viability of the rehydrated biofilm composition was then evaluated by determining the CFU per composition. First each rehydrated biofilm composition was vortexed (30 seconds) and sonicated (45 kHz for approximately 30 seconds). This step was repeated twice. Each sample was then serially diluted in Standard Method Dilution Water (SMDW—0.0425 g/L KH₂PO₄ and 0.405 g/L MgCl₂) and spread plated onto trypticase soy agar (TSA). The plated samples were then incubated at 37° C. for 24±4 hours. The agar plates were then counted and CFU per composition was calculated. The mean log density was calculated to be 7.7 log CFU per composition.

### Example 3. Stability of Dehydrated Biofilm Compositions Over Time

A study was conducted over 2 months to determine if exemplary Pseudomonas aeruginosa and Staphylococcus aureus dehydrated biofilm compositions could maintain stability over a significant period of time. In particular, the study aimed to investigate if the dehydrated biofilm compositions were capable of retaining viability and biofilm phenotype over time, including extracellular matrix integrity and substrate attachment.

Dehydrated biofilm compositions comprising Pseudomonas aeruginosa ATCC 15442 attached to a K1 carrier (10×7 mm) and further comprising a mixture of stabilizing agents were prepared. The compositions were sealed in a 15×22 cm foil pouch with low permeability to moisture and oxygen, and an oxygen scavenger and desiccant were included in the container. The dehydrated biofilm compositions were then stored at refrigeration temperature 4-8° C.

Dehydrated biofilm compositions comprising Staphylococcus aureus ATCC 6538 attached to a K1 carrier (10×7 mm) and further comprising a mixture of stabilizing agents were also prepared. The compositions were sealed in a 15×22 cm foil pouch with low permeability to moisture and oxygen, and stored at refrigeration temperature 4-8° C.

TABLE 1
Stability of a Pseudomonas aeruginosa dehydrated
biofilm composition

| Time Point After | Log CFU Per | Mean Log CFU Per |
|---|---|---|
| Dehydration | Composition | Composition |
| 0 days | 8.6 | 8.6 |
| | 8.7 | |
| | 8.4 | |
| 7 days | 8.6 | 8.4 |
| | 8.2 | |
| | 8.4 | |
| 14 days | 8.7 | 8.5 |
| | 8.5 | |
| | 8.5 | |
| 30 days | 8.8 | 8.7 |
| | 8.6 | |
| | 8.6 | |
| 60 days | 8.6 | 8.7 |
| | 8.7 | |
| | 8.7 | |

The mean log density of the Pseudomonas aeruginosa biofilm composition before dehydration was determined to be 9.3 log CFU. Surprisingly, the dehydrated biofilm composition demonstrated no loss of viability over a 60 day period. Furthermore, the dehydrated biofilm composition demonstrated a loss of only about 0.6 log CFU from the dehydration process relative to the mean log density of the Pseudomonas aeruginosa biofilm composition before dehydration. At each time point in the stability study, the composition was examined for retention of substrate attachment and overall integrity of the extracellular matrix upon rehydration. At each time point the rehydrated composition demonstrated biofilm substrate attachment and extracellular matrix integrity.

TABLE 2
Stability of a Staphylococcus aureus dehydrated
biofilm composition

| Time Point After | Log CFU Per | Mean Log CFU Per |
|---|---|---|
| Dehydration | Composition | Composition |
| 0 days | 8.0 | 7.9 |
| | 7.8 | |
| | 7.8 | |
| 7 days | 7.7 | 7.8 |
| | 7.8 | |
| | 7.9 | |
| 14 days | 8.0 | 7.9 |
| | 8.1 | |
| | 7.7 | |
| 30 days | 7.8 | 7.8 |
| | 7.9 | |
| | 8.0 | |
| 60 days | 7.9 | 7.9 |
| | 7.7 | |
| | 8.1 | |

The mean log density of the Staphylococcus aureus biofilm composition before dehydration was determined to be 8.0 log CFU. Surprisingly, the dehydrated biofilm composition demonstrated no loss of viability over a 60 day period. Furthermore, the dehydrated biofilm composition demonstrated a loss of only about 0.1 log CFU from the dehydration process relative to the mean log density of the Pseudomonas aeruginosa biofilm composition before dehydration. At each time point in the stability study, the composition was examined for retention of substrate attachment and overall integrity of the extracellular matrix upon rehydration. At each time point the rehydrated composition demonstrated biofilm substrate attachment and extracellular matrix integrity.

### Example 4. Performance of Dehydrated Biofilm Compositions in Anti-biofilm Efficacy Testing

A study was conducted to evaluate the performance of exemplary dehydrated biofilm compositions in anti-biofilm efficacy testing after rehydration.

Dehydrated biofilm compositions comprising Pseudomonas aeruginosa ATCC 15442 attached to a K1 carrier (10×7 mm) and further comprising a mixture of stabilizing agents were prepared. The compositions were sealed in a 15×22 cm foil pouch with low permeability to moisture and oxygen, and an oxygen scavenger and desiccant were included in the container. The dehydrated biofilm compositions were then stored at refrigeration temperature 4-8° C. On the day of testing, the dehydrated biofilm compositions were removed from the foil pouch and placed into a container. The dehydrated biofilm compositions were then submerged in 2 mL of a rehydration solution (pH 7.4) and maintained in the solution for 20 minutes at room temperature to allow for rehydration of the biofilm composition. Disinfectant testing was then conducted in triplicate and followed the general methodology from EPA MLB SOP MB-20.

TABLE 3
Anti-biofilm Efficacy Testing of a Quaternary Alcohol
Product
Control

| | Log | Mean Log | Standard |
|---|---|---|---|
| Replicate | CFU | CFU | Deviation |
| #1 | 8.3 | 8.2 | 0.1 |
| #2 | 8.2 | | |
| #3 | 8.2 | | |

Low Concentration

| | | | | | |
|---|---|---|---|---|---|
| | Log | Mean Log | Standard | Log CFU | Mean Log |
| Replicate | CFU | CFU | Deviation | Loss | CFU Loss |
| #1 | 5.0 | 5.1 | 0.3 | 3.3 | 3.1 |
| #2 | 5.5 | 2.8 | | | |
| #3 | 4.9 | 3.4 | | | |

High Concentration

| | | | | | |
|---|---|---|---|---|---|
| | Log | Mean Log | Standard | Log CFU | Mean Log |
| Replicate | CFU | CFU | Deviation | Loss | CFU Loss |
| #1 | 0 | 0 | n/a | 8.2 | 8.2 |
| #2 | 0 | 8.2 | | | |
| #3 | 0 | 8.2 | | | |

TABLE 4
Anti-biofilm Efficacy Testing of a Sodium Hypochlorite
Product
Control

| | Log | Mean Log | Standard |
|---|---|---|---|
| Replicate | CFU | CFU | Deviation |
| #1 | 8.3 | 8.5 | 0.2 |
| #2 | 8.7 | | |
| #3 | 8.6 | | |

Low Concentration

| | | | | | |
|---|---|---|---|---|---|
| | Log | Mean Log | Standard | Log CFU | Mean Log |
| Replicate | CFU | CFU | Deviation | Loss | CFU Loss |
| #1 | 8.5 | 7.5 | 1.1 | 0 | 1.1 |
| #2 | 6.2 | | | 2.3 | |
| #3 | 7.6 | | | 0.9 | |

High Concentration

| | | | | | |
|---|---|---|---|---|---|
| | Log | Mean Log | Standard | Log CFU | Mean Log |
| Replicate | CFU | CFU | Deviation | Loss | CFU Loss |
| #1 | 0 | 0 | n/a | 8.5 | 8.5 |
| #2 | 0 | 0 | | 8.5 | |
| #3 | 0 | 0 | | 8.5 | |

The rehydrated biofilm samples demonstrated responsiveness to changes in disinfectant concentration for both the quaternary alcohol disinfectant product and the sodium hypochlorite disinfectant product. The results of the study indicate that the dehydrated biofilm compositions can be rehydrated and successfully utilized in anti-biofilm efficacy testing to provide meaningful results.

### Example 5. Evaluation of Dehydrated Biofilm Composition Performance in Anti-biofilm Efficacy Testing Compared to Fresh Biofilm Samples

A study was conducted to evaluate the performance of exemplary Pseudomonas aeruginosa dehydrated biofilm compositions in anti-biofilm efficacy testing as compared to fresh Pseudomonas aeruginosa biofilm samples (non-dehydrated biofilm samples). The United States Environmental Protection Agency is responsible for the approval of disinfectants in the United States that are used on inanimate objects, e.g., in a food production facility. In order to gain approval for anti-biofilm disinfectant product claims, the product must exhibit a 6 log CFU reduction of viable microbes in biofilms as tested according to EPA MLB SOP MB-20. Using a 6 log CFU reduction of viable microbes as the means for comparison, this study evaluated the agreement of disinfectant performance as tested against fresh biofilm and dehydrated biofilm compositions that were rehydrated.

Dehydrated biofilm compositions comprising Pseudomonas aeruginosa ATCC 15442 attached to a substrate and further comprising a mixture of stabilizing agents were prepared. Prior to testing, dehydrated biofilm compositions were submerged in 2 mL of a rehydration solution (pH 7.4) and maintained in the solution for 20 minutes at room temperature to allow for rehydration of the biofilm composition. The fresh biofilm samples were cultured under the same conditions and using the same protocol as the dehydrated biofilm samples. For each comparison, the anti-biofilm efficacy testing was conducted immediately after the culturing the fresh biofilm samples was finished.

The disinfectants tested included four sodium hypochlorite-based disinfectants, two alcohol-based disinfectants, and two phenol-based disinfectants. “Not Pass” indicates that the mean log reduction of viable cells was <6 log CFU for the respective disinfectant test. “Pass” indicates that the mean log reduction of viable cells was >6 log CFU for the respective disinfectant test. If both the fresh biofilm sample and the rehydrated biofilm composition exhibited the same “Pass” or “No Pass” result for a disinfectant, they were considered in agreement. Testing was conducted in triplicate and followed the general methodology of EPA MLB SOP MB-20.

TABLE 5
Evaluation of Agreement Between Fresh Biofilm Samples and
Dehydrated Biofilm Compositions in Anti-biofilm Efficacy Testing

| | Fresh Biofilm | Dehydrated Biofilm | Agreement |
|---|---|---|---|
| | Sample | Composition | (Yes/No) |
| Disinfectant #1 | Not Pass | Not Pass | Yes |
| Disinfectant #2 | Not Pass | Not Pass | Yes |
| Disinfectant #3 | Pass | Pass | Yes |
| Disinfectant #4 | Pass | Pass | Yes |
| Disinfectant #5 | Not Pass | Not Pass | Yes |
| Disinfectant #6 | Not Pass | Not Pass | Yes |
| Disinfectant #7 | Not Pass | Not Pass | Yes |
| Disinfectant #8 | Not Pass | Not Pass | Yes |

For each of the disinfectants tested, the rehydrated biofilm samples demonstrated agreement with the fresh biofilm samples. The results of the study indicate that the dehydrated biofilm compositions can be rehydrated and successfully utilized in anti-biofilm efficacy testing to provide meaningful results as compared to fresh biofilm samples.

### Example 6

Twenty-four dehydrated biofilm compositions are prepared as in Example 1. A composition is placed into each well of a 24-well microplate. The compositions are enclosed in the microplate and the microplate is then sealed in a Mylar® bag with 10 g of a desiccant and 500 cc of an oxygen scavenger. The Mylar® bag and its content are then stored at about 4° C.

### Example 7

The composition of Example 6 and a bottle of rehydration solution (45 mL of 10 mM TRIS buffer) are further sealed in a container.

### Example 8

Twenty-four dehydrated biofilm compositions are prepared as in Example 2. A composition is placed into each well of a 24-well microplate. The compositions are enclosed in the microplate and the microplate is then sealed in a Mylar® bag with no desiccant and no oxygen scavenger. The Mylar® bag and its contents are then stored at about 4° C.

### Example 9

The composition of Example 8 and a bottle of rehydration solution (45 mL of 10 mM TRIS buffer) are further sealed in a container.

### Example 10

Eight dehydrated biofilm compositions are prepared as in Example 1. Each composition is placed into a sterile vial and 0.5 mL of rehydration solution is dispensed into the vial. Each composition is submerged in the rehydration solution for about 20 minutes at about 21° C. Each composition is then separated from the rehydration solution and placed into a sterile 50 mL conical tube. Five replicates are used as treated samples and 3 replicates are used as control samples. The treated samples are contacted with 4 mL of a test substance and the control samples are contacted with 4 mL a control substance (dilution buffer). The contact time for both the treated samples and control samples is 10 minutes at about 21° C. At the end of the contact time, 36 mL of an appropriate neutralizer is added to each tube.

After neutralization, the anti-biofilm efficacy of the test substance is determined. The contents of each tube are sufficiently agitated to ensure that the biofilm is unattached from the substrate. Viability of the treated samples and control samples is assessed by CFU enumeration.

### Example 11

Twenty-four dehydrated biofilm compositions comprising between 1×10⁸ CFU and 1×10^{9.5} CFU Pseudomonas aeruginosa ATCC 15442 secured to a Kaldnes-type carrier, 60 mg trehalose, 25 mg ascorbic acid, and 4% residual water content are enclosed in a 24-well microplate. The microplate is sealed in a Mylar® bag with 10 g desiccant and 500 cc oxygen scavenger. The Mylar® bag and its contents are then stored in a refrigerator at about 4° C. for about 90 days.

The Mylar® bag is then removed from the refrigerator and brought to room temperature. The Mylar® bag is opened and the microplate is removed. The lid is removed from the microplate and each dehydrated biofilm composition is contacted by 0.5 mL of a rehydration solution. Each composition is submerged in the rehydration solution for about 20 minutes at about 21° C. Each composition is then separated from the rehydration solution and placed into a sterile 50 mL conical tube. Four test substances are evaluated. For each test substance, 5 replicates are used as treated samples. Four replicates are used as control samples. The treated samples are contacted with 4 mL of a test substance and the control samples are contacted with 4 mL a control substance (dilution buffer). The contact time for both the treated samples and control samples is 10 minutes at about 21° C. At the end of the contact time, 36 mL of an appropriate neutralizer is added to each tube.

After neutralization, the anti-biofilm efficacy of the test substances is determined. The contents of each tube are sufficiently agitated to ensure that the biofilm is unattached from the substrate. Viability of the treated samples and control samples is assessed by CFU enumeration.

### Example 12

Twenty-four dehydrated biofilm compositions comprising between 1×10^{7.5} CFU and 1×10⁹ CFU Staphylococcus aureus ATCC 6538 secured to a Kaldnes-type carrier, 40 mg lactose, 20 mg ascorbic acid, and 2.5% residual water content are enclosed in a 24-well microplate. The microplate is sealed in a Mylar® bag and stored in a refrigerator at about 4° C. for about 90 days.

The Mylar® bag is then removed from the refrigerator and brought to room temperature. The Mylar® bag is opened and the microplate is removed. The lid is removed from the microplate and each dehydrated biofilm composition is contacted by 0.5 mL of a rehydration solution. Each composition is submerged in the rehydration solution for about 20 minutes at about 21° C. Each composition is then separated from the rehydration solution and placed into a sterile 50 mL conical tube. Four test substances are evaluated. For each test substance, 5 replicates are used as treated samples. Four replicates are used as control samples. The treated samples are contacted with 4 mL of a test substance and the control samples are contacted with 4 mL a control substance (dilution buffer). The contact time for both the treated samples and control samples is 10 minutes at about 21° C. At the end of the contact time, 36 mL of an appropriate neutralizer is added to each tube.

After neutralization, the anti-biofilm efficacy of the test substances is determined. The contents of each tube are sufficiently agitated to ensure that the biofilm is unattached from the substrate. Viability of the treated samples and control samples is assessed by CFU enumeration.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A dehydrated biofilm assembly for use in antimicrobial testing, the dehydrated biofilm assembly comprising:
a substrate;
a dehydrated biofilm secured to the substrate;
wherein the dehydrated biofilm includes a viable population of micro-organisms; and
wherein the dehydrated biofilm is configured to retain securement to the substrate and exhibit a biofilm phenotype upon rehydration.

2. The dehydrated biofilm assembly of claim 1, wherein the viable population of micro-organisms is disposed in a matrix.

3. The dehydrated biofilm assembly of any one of claims 1-2, wherein the viable population of micro-organisms comprises a viable cell count in the range of about 1×10⁶ CFU and about 1×10¹⁰ CFU of micro-organisms.

4. The dehydrated biofilm assembly of any one of claims 1-3, wherein the viable population of micro-organisms comprises Pseudomonas aeruginosa.

5. The dehydrated biofilm assembly of claim 4, the viable population of micro-organisms comprises a viable cell count in the range of about 1×10⁸ CFU and about 1×10^{9.5} CFU Pseudomonas aeruginosa.

6. The dehydrated biofilm assembly of any one of claims 1-5, wherein the viable population of micro-organisms comprises Staphylococcus aureus.

7. The dehydrated biofilm assembly of claim 6, the viable population of micro-organisms comprises a viable cell count in the range of about 1×10^{7.5} CFU and about 1×10⁹ CFU Staphylococcus aureus.

8. The dehydrated biofilm assembly of any one of claims 1-7, wherein the dehydrated biofilm includes a stabilizing agent.

9. The dehydrated biofilm assembly of claim 8, wherein the stabilizing agent includes a sugar.

10. The dehydrated biofilm assembly of any one of claims 8-9, wherein the stabilizing agent includes a non-reducing sugar.

11. The dehydrated biofilm assembly of any one of claims 8-11, wherein the stabilizing agent includes sucrose.

12. The dehydrated biofilm assembly of any one of claims 8-11, wherein the stabilizing agent includes an antioxidant.

13. The dehydrated biofilm assembly of any one of claims 8-12, wherein the stabilizing agent includes ascorbic acid.

14. The dehydrated biofilm assembly of any one of claims 8-13, wherein the stabilizing agent includes a buffer.

15. The dehydrated biofilm assembly of any one of claims 8-14, wherein the stabilizing agent includes Tris(hydroxymethyl)aminomethane.

16. The dehydrated biofilm assembly of any one of claims 1-15, wherein the dehydrated biofilm has a residual water content in the range of about 0-10% by weight of the dehydrated biofilm.

17. The dehydrated biofilm assembly of any one of claims 1-15, wherein the dehydrated biofilm has a residual water content in the range of about 0-7.5% by weight of the dehydrated biofilm.

18. The dehydrated biofilm assembly of any one of claims 1-17, wherein the dehydrated biofilm has a loss of viability of less than about 3 log CFU over a period of 30 days at a temperature of 5° C.

19. The dehydrated biofilm assembly of any one of claims 1-17, wherein the dehydrated biofilm has a loss of viability of less than about 3 log CFU over a period of 60 days at a temperature of 5° C.

20. The dehydrated biofilm assembly of any one of claims 1-17, wherein the dehydrated biofilm has a loss of viability of less than about 3 log CFU over a period of 90 days at a temperature of 5° C.

21. The dehydrated biofilm assembly of any one of claims 1-17, wherein the dehydrated biofilm has a loss of viability of less than about 0.5 log CFU over a period of 30 days at a temperature of 5° C.

22. The dehydrated biofilm assembly of any one of claims 1-17, wherein the dehydrated biofilm has a loss of viability of less than about 0.5 log CFU over a period of 60 days at a temperature of 5° C.

23. The dehydrated biofilm assembly of any one of claims 1-17, wherein the dehydrated biofilm has a loss of viability of less than about 0.5 log CFU over a period of 90 days at a temperature of 5° C.

24. The dehydrated biofilm assembly of any one of claims 1-23, wherein the dehydrated biofilm and the substrate are disposed within a primary container.

25. The dehydrated biofilm assembly of claim 24, wherein the primary container is sealed within a secondary container that has low permeability to moisture and oxygen.

26. The dehydrated biofilm assembly of claim 25, wherein the primary container, the secondary container, or both are impermeable to at least one of moisture and oxygen.

27. The dehydrated biofilm assembly of any one of claims 25-26, further comprising a desiccant disposed within the secondary container.

28. The dehydrated biofilm assembly of any one of claims 25-27, further comprising an oxygen scavenger disposed within the secondary container.

29. A method for testing the anti-biofilm efficacy of a test substance, the method comprising:
submerging a dehydrated biofilm assembly in an aqueous solution, the dehydrated biofilm assembly including a dehydrated biofilm secured to a substrate;
disposing the dehydrated biofilm assembly in the aqueous solution for a rehydration time to form a rehydrated biofilm;
contacting the rehydrated biofilm with a test substance for a contact time;
contacting the rehydrated biofilm and the test substance with a neutralizer;
agitating the rehydrated biofilm; and
observing an indication of anti-biofilm efficacy.

30. The method of claim 29, wherein observing an indication of anti-biofilm efficacy includes quantitative assessment.

31. The method of claim 30, wherein quantitative assessment includes determining a loss of viable cells in the rehydrated biofilm.

32. The method of claim 30, wherein quantitative assessment includes utilizing a stain or a dye.

33. The method of any one of claims 29-32, wherein observing an indication of anti-biofilm efficacy includes qualitative assessment.

34. The method of any one of claims 29-33, wherein the rehydration time is about 1-40 minutes.

35. The method of any one of claims 29-33, wherein the rehydration time is about 1-25 minutes.

36. The method of any one of claims 29-35, wherein the test substance includes a disinfectant.

37. The method of any one of claims 29-36, wherein the contact time is about 1-10 minutes.

38. The method of any one of claims 29-37, wherein the dehydrated biofilm assembly is disposed in a container and wherein disposing the dehydrated biofilm assembly in the aqueous solution for a rehydration time to form a rehydrated biofilm further includes removing the rehydrated biofilm from the container.

39. A method for manufacturing a dehydrated biofilm assembly for use in antimicrobial testing, the method comprising:
culturing a biofilm on a substrate;
submerging the biofilm in a solution that includes a stabilizing agent;
freezing the biofilm in the solution;
lyophilizing the biofilm to dehydrate the biofilm;
disposing the dehydrated biofilm and the substrate in a primary container; and
sealing the primary container within a secondary container that has low permeability to moisture and oxygen.

40. The method of claim 39, wherein the primary container, the secondary container, or both are impermeable to at least one of moisture and oxygen.

41. A kit for evaluating the anti-biofilm efficacy of a test substance comprising:
a dehydrated biofilm composition comprising a microbial cell population enmeshed in a self-produced extracellular polymeric matrix and attached to a substrate, wherein the composition retains substrate attachment and exhibits a viable cell count and a biofilm phenotype upon rehydration,
wherein the viable cell count is about 1×10⁶ CFU and about 1×10¹⁰ CFU upon rehydration of the dehydrated biofilm composition,
wherein the dehydrated biofilm composition comprises a stabilizing agent,
wherein the dehydrated biofilm composition comprises a residual water content of between about 0% to about 10% by weight of the dehydrated biofilm composition,
wherein the dehydrated biofilm composition has a loss of viability of less than 3 log CFU over a period of about 90 days at a temperature of about 5° C.,
wherein the dehydrated biofilm composition is disposed within a primary, container, and
wherein the primary container is sealed within a secondary container that has low permeability to moisture and oxygen.
